(19) United States
(12) Patent Application Publication
(10) Pub. No.: US 2026/0318921 A1
(43) Pub. Date: Oct. 15, 2026

(54) POST-REPERFUSION CARDIAC TROPONIN KINETICS AS A DIAGNOSTIC BIOMARKER OF HEMORRHAGIC MYOCARDIAL INFARCTION

(71) Applicant: THE TRUSTEES OF INDIANA UNIVERSITY, Bloomington, IN (US)

(72) Inventors: Rohan DHARMAKUMAR, Zionsville, IN (US); Keyurkumar P. VORA, Carmel, IN (US)

(21) Appl. No.: 19/483,398

(22) PCT Filed: May 10, 2024
(86) PCT No.: PCT/US2024/028883

§ 371 Date: Nov. 11, 2025

Related U.S. Application Data
(60) Provisional application No. 63/465,802, filed on May 11, 2023.
(60) Provisional application No. 63/621,216, filed on Jan. 16, 2024.

(51) Int. Cl. G01N 33/68

(52) U.S. Cl. CPC G01N 33/6887; G01N 2333/4712; G01N 2800/324; G01N 2800/52

(57) ABSTRACT

Post-reperfusion high-sensitivity troponin-I kinetics are used as a clinical diagnostic biomarker and a highly sensitive method for time-sensitive diagnosis of intramyocardial hemorrhage-driven ischemia-reperfusion injury. Treatment methods are also provided for patients indicated with the intramyocardial hemorrhage as measured by the troponin-I kinetics.

### CROSS-REFERENCE TO RELATED APPLICATIONS

This application includes a claim of priority under 35 U.S.C. § 119(e) to U.S. provisional patent application No. 63/465,802, filed May 11, 2023, and U.S. provisional patent application No. 63/621,216, filed Jan. 16, 2024, the entirety of both of which is hereby incorporated by reference.

### STATEMENT REGARDING FEDERALLY SPONSORED RESEARCH OR DEVELOPMENT

This invention was made with Government support under grant nos. HL133407, HL136578, and HL147133 awarded by the National Institutes of Health. The Government has certain rights in the invention.

### FIELD OF INVENTION

This invention relates to diagnosis and treatment of patients with post-reperfusion intramyocardial hemorrhage (IMH).

### BACKGROUND

Heart attacks are caused due to a blockage in blood vessels that supply blood to heart muscles. Current medical practice involves restoring the blocked blood supply to heart muscles which is known as reperfusion. However, reperfusion therapy is a double-edge sword; while its benefits are undeniable, it is known to impart additional injury to the heart muscle (myocardium) and contribute towards long-term complications, notably chronic heart failure (CHF) that drives major adverse cardiovascular events (MACE). Centers for Disease Control reports that >300,000 deaths/year are attributable to CHF in the US. Although the extent of myocardial damage (MI size) is a long-established predictor of CHF, recent advances in imaging have shown that intramyocardial hemorrhage (IMH; bleeding within the heart muscle), a potential consequence of reperfusion therapy for MI, is a major predictor of MACE. In fact, several studies have shown that hemorrhagic MI (hMI) patients are at >2-fold greater risk for MACE than those without hemorrhage. Hemorrhagic transformation of myocardial infarction (MI) following reperfusion can rapidly diminish salvaged myocardium within the first 24 hours and accelerate adverse remodeling precipitating chronic heart failure.

Currently the most prevalent means for detection of IMH in the heart following reperfusion is via cardiac MRI (CMR). However, CMR has several major limitations: it is only effectively performed 3-5 days after reperfusion due to limited sensitivity at earlier time points; the MI size can double within the 12 hours after reperfusion therapy in hMI patients but not in non-hMI patients, and hence the inability to detect hMIs in a timely fashion impairs identification, development, or testing of new therapies uniquely targeting hMI; and CMR is expensive, not widely available in non-urban settings and challenging to perform.

Therefore, it is an object of the present invention to develop readily accessible, simple and robust, early Point-of-Care (POC) detection methods for monitoring patients with ischemic heart disease especially after reperfusion of the ischemic myocardium, and/or for identification/diagnosis of IMH.

It is another object of the present invention to provide treatment methods to patients after a reperfusion therapy for myocardial ischemia based on detected markers indicative of IMH.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### SUMMARY OF THE INVENTION

The following embodiments and aspects thereof are described and illustrated in conjunction with compositions and methods which are meant to be exemplary and illustrative, not limiting in scope.

Methods are provided for treating a human subject in need thereof, especially a human subject having undergone a reperfusion therapy following myocardial ischemia. In some embodiments, methods are provided for treating a human subject inflicted with myocardial infarction who is indicated to have hemorrhage, especially after a reperfusion therapy (e.g., percutaneous coronary intervention). In various embodiments, a human subject is indicated to have hemorrhage (hemorrhagic myocardial infarction) if his blood level of troponin-I is greater than the threshold shown in Table 2 at different hours after receiving a reperfusion therapy.

In various embodiments, the methods include administering a treatment therapy (e.g., an iron chelator, an agent that binds or regulates heme, and/or an anti-inflammatory agent) to the human subject detected with one or more of:
a level of troponin-I being at least 90 ng/mL in a blood sample obtained between 0.5 hour and 7.5 hours after the reperfusion therapy, optionally at least 250 ng/mL in a blood sample obtained between 3-5 hours after the reperfusion therapy,
a level of the troponin-I being at least 75 ng/mL in a blood sample obtained between 7.5 hours and 11.5 hours after the reperfusion therapy,
a level of the troponin-I being at least 60 ng/mL in a blood sample obtained between 11.5 hours and 16.5 hours after the reperfusion therapy,
a level of the troponin-I being at least 40 ng/mL in a blood sample obtained between 16.5 hours and 20.5 hours after the reperfusion therapy, and
a level of the troponin-I being at least 30 ng/mL in a blood sample obtained between 20.5 hours and 24.5 hours after the reperfusion therapy.

In various implementations, the detection of said one or more levels of the troponin-I at said one or more times indicates the human subject has hemorrhagic myocardial infarction after the reperfusion therapy, and hence the treatment therapy is to treat, mitigate, or reduce hemorrhagic myocardial infarction.

Other methods for treating a human subject in need thereof are also provided, wherein the human subject has undergone or undergoes a reperfusion therapy following myocardial ischemia, and the methods include: administering a treatment therapy (e.g., an iron chelator, an agent that binds or regulates heme, and/or an anti-inflammatory agent) to the human subject detected with a high level of troponin-I in a blood sample obtained between 3 hours and 5 hours following the reperfusion therapy (e.g., about 4 hours after the reperfusion therapy, or between 3.5 and 4.5 hours after the reperfusion therapy), wherein the high level of the troponin-I is one or both of: (i) at least 300 ng/mL or within 348±37 ng/mL, and (ii) a concentration at least 10 times that obtained between 9.5 and 11.5 hours after a reperfusion therapy in a control human subject, wherein the control human subject does not have hemorrhagic myocardial infarction after the reperfusion therapy following a myocardial infarction.

In various implementations, the detection of said one or both high levels of the troponin-I between 3 hours and 5 hours following the reperfusion therapy indicates the human subject has hemorrhagic myocardial infarction after the reperfusion therapy, and the treatment therapy is to treat, mitigate, or reduce hemorrhagic myocardial infarction.

Methods are further provided for treating a human subject in need thereof, wherein the human subject undergoes a reperfusion therapy following myocardial ischemia, and the methods include: administering a treatment therapy (e.g., an iron chelator, an agent that binds or regulates heme, and/or an anti-inflammatory agent) to the human subject detected with a high rate of increase in troponin-I level comparing within first 2 hours after the reperfusion therapy (e.g., at about 1 hour after the reperfusion therapy) relative to before the reperfusion therapy, preferably comparing within first 2 hours after the reperfusion therapy relative to within one hour or 30 minutes or 10 minutes before the reperfusion therapy, wherein the high rate of increase is: being at least 190 ng/mL/hr or within 218.55±26.45 ng/mL/hr, and/or being at least 10 times a rate of increase in a control human subject, wherein the rate of increase in the control human subject is comparing within 21 hours after a reperfusion therapy relative to immediately after the reperfusion therapy in the control human subject, and wherein the control human subject does not have hemorrhagic myocardial infarction after the reperfusion therapy following myocardial infarction.

In various implementations, the detection of said one or both high rate of increase in the troponin-I between 0.5 hour and 2 hours following the reperfusion therapy indicates the human subject has hemorrhagic myocardial infarction after the reperfusion therapy, and the treatment therapy is to treat, mitigate, or reduce hemorrhagic myocardial infarction.

Other embodiments provide methods of treating a canine subject in need thereof, wherein the canine subject undergoes a reperfusion therapy following myocardial ischemia, and the methods include: administering a treatment therapy (e.g., an iron chelator, an agent that binds or regulates heme, and/or an anti-inflammatory agent) to a canine subject detected with:
a level of troponin-I being at least 200 ng/mL (e.g., at least 240 ng/mL) or within 259.72±15.44 ng/mL in a blood sample obtained or in blood measured between 3 hours and 4 hours after the reperfusion,
a level of the troponin-I in a blood sample obtained or in blood measured between 3 hours and 4 hours after the reperfusion being at least 3.5 times that obtained between 15 hours and 19 hours after a reperfusion therapy in a control canine subject, wherein the control canine subject does not have hemorrhagic myocardial infarction after the reperfusion therapy following a myocardial infarction, and/or
a high rate of increase in the troponin-I comparing a blood sample obtained between 1.5 hours and 2 hours after the reperfusion relative to one immediately after the reperfusion therapy, wherein the high rate of increase is at least 60 ng/mL/hr and/or at least 10 times a rate of increase in the control canine subject, wherein the rate of increase in the control canine subject is comparing a blood sample obtained between 4 hours and 9 hours after the reperfusion therapy in the control canine subject relative to one immediately after the reperfusion therapy in the control canine subject.

In various implementations, the detection of said one or both levels of the troponin-I and/or said high rate of increase in the troponin-I indicates the canine subject has hemorrhagic myocardial infarction after the reperfusion therapy.

In some embodiments, the treatment therapy is administered immediately after detecting the level of the troponin-I indicating the presence of the hemorrhagic myocardial infarction in the subject, or administered within 3 hours after the detection.

In some embodiments, the treatment therapy comprises the iron chelator, and the iron chelator comprises a ferrous iron chelator, a ferric iron chelator, or both.

Further provided include methods of examining a subject in need thereof, wherein the subject has myocardial ischemia and received a reperfusion therapy, and the methods include measuring a level of the troponin-I of the subjects at one or more time points between immediately following the reperfusion therapy and 24 hours following the reperfusion therapy from the subject. For example, the measuring comprises measuring in the blood samples at two or more time points between immediately following the reperfusion therapy and 4 hours following the reperfusion therapy, to obtain a rate of increase in the troponin-I level over the time lapse between the two or more time points. As another example, the measuring comprises measuring a time series of troponin-I levels in the blood samples obtained or measured noninvasively in the subject at three or more time points, and obtaining a highest level of the troponin-I from the time series.

In the methods of examining a human subject, the human subject is indicated to have hemorrhagic myocardial infarction after the reperfusion therapy when the measuring detects:
a level of the troponin-I being at least 90 ng/mL in a blood sample obtained or measured noninvasively from the subject between 0.5 hour and 7.5 hours after the reperfusion therapy,
a level of the troponin-I being at least 75 ng/mL in a blood sample obtained or measured noninvasively from the subject between 7.5 hours and 11.5 hours after the reperfusion therapy,
a level of the troponin-I being at least 60 ng/mL in a blood sample obtained or measured noninvasively from the subject between 11.5 hours and 16.5 hours after the reperfusion therapy,
a level of the troponin-I being at least 40 ng/mL in a blood sample obtained or measured noninvasively from the subject between 16.5 hours and 20.5 hours after the reperfusion therapy,
a level of the troponin-I being at least 30 ng/mL in a blood sample obtained or measured noninvasively from the subject between 20.5 hours and 24.5 hours after the reperfusion therapy,
a high level of the troponin-I being at least 300 ng/mL or within 348±37 ng/mL in a blood sample obtained or measured noninvasively from the subject between 3 hours and 5 hours after the reperfusion therapy,
a high level of the troponin-I in a blood sample obtained or measured noninvasively from the subject between 3 hours and 5 hours after the reperfusion therapy being at least 10 times that obtained or measured between 9.5 and 11.5 hours after a reperfusion therapy in a control human subject,
a high rate of increase in the troponin-I level comparing a blood sample obtained or measured noninvasively from the subject within 2 hours after the reperfusion therapy relative to one immediately after the reperfusion therapy, wherein the high rate of increase is at least 190 ng/mL/hr, or within 218.55±26.45 ng/mL/hr, and/or being at least 10 times a rate of increase in the control human subject, wherein the rate of increase in the control human subject is comparing within 21 hours after the reperfusion therapy relative to one immediately after the reperfusion therapy in the control human subject,
wherein the control human subject is a human who does not have hemorrhagic myocardial infarction after the reperfusion therapy following myocardial infarction.

Following an examination, a treatment therapy may be prescribed or administered to the human subject indicated to have hemorrhagic myocardial infarction.

Additionally for a canine subject, it is indicated to have hemorrhagic myocardial infarction when the measuring detects:
a level of the troponin-I being at least 200 ng/mL or within 259.72±15.44 ng/mL in a blood sample obtained or measured noninvasively from the subject between 3 hours and 4 hours after the reperfusion,
a level of the troponin-I in a blood sample obtained or measured noninvasively from the subject between 3 hours and 4 hours after the reperfusion being at least 3.5 times that between 15 hours and 19 hours after a reperfusion therapy in a control canine subject, wherein the control canine subject does not have hemorrhagic myocardial infarction after the reperfusion therapy following a myocardial infarction, and/or
a high rate of increase in the troponin-I comparing a blood sample obtained or measured noninvasively from the subject between 1.5 hours and 2 hours after the reperfusion relative to one immediately after the reperfusion therapy, wherein the high rate of increase is at least 60 ng/mL/hr and/or at least 10 times a rate of increase in the control canine subject, wherein the rate of increase in the control canine subject is comparing a blood sample obtained between 4 hours and 9 hours after the reperfusion therapy in the control canine subject relative to one obtained immediately after the reperfusion therapy in the control canine subject, and optionally a treatment therapy is prescribed or administered to the canine subject.

In various embodiments, the troponin-I is cardiac troponin-I or a fragment thereof. Troponin-I may be measured or detected using a monoclonal antibody specifically binding cardiac troponin-I or with available quantitation assay kits.

Further embodiments provide methods for providing prognosis of a likelihood of survival about 30 days after receiving reperfusion therapy in a human subject with acute ST-elevation myocardial infarction (STEMI), and/or providing prognosis of a likelihood of requiring rehospitalization within about 30 days from receiving the reperfusion therapy in the human subject, and the methods include: measuring a level of troponin-I in a sample (e.g., blood sample) of the subject, at one or more time points following the reperfusion therapy, wherein:
a level of troponin-I being at least 90 ng/mL in a blood sample obtained between 0.5 hour and 7.5 hours after the reperfusion therapy,
a level of the troponin-I being at least 75 ng/mL in a blood sample obtained between 7.5 hours and 11.5 hours after the reperfusion therapy,
a level of the troponin-I being at least 60 ng/mL in a blood sample obtained between 11.5 hours and 16.5 hours after the reperfusion therapy,
a level of the troponin-I being at least 40 ng/mL in a blood sample obtained between 16.5 hours and 20.5 hours after the reperfusion therapy,
a level of the troponin-I being at least 30 ng/mL in a blood sample obtained between 20.5 hours and 24.5 hours after the reperfusion therapy,
a level of the troponin-I being at least 300 ng/mL or within 348±37 ng/mL in a blood sample obtained between 3 hours and 5 hours after the reperfusion therapy, and/or
a level of the troponin-I in a blood sample obtained between 3 hours and 5 hours after the reperfusion therapy being at least 10 times that obtained between 9.5 and 11.5 hours after a reperfusion therapy in a control human subject, wherein the control human subject does not have hemorrhagic myocardial infarction after the reperfusion therapy following a myocardial infarction,
indicates that the human subject has a lower likelihood of survival at about the 30 days and a higher likelihood of requiring the rehospitalization within the about 30 days of the reperfusion, compared to the control human subject.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIGS. 1A-1C depict representative reperfused STEMI patients with and without hemorrhagic MI, respectively. FIG. 1A shows coronary angiograms illustrating the culprit coronary artery occlusions. FIG. 1B shows the differential time-resolved post-PCI [hs-cTn-I]including pre-PCI, immediately after PCI, and hourly measurements up to 12 hours post-PCI, with additional readings at 20-, 24-, and 48-hours post-PCI. FIG. 1C shows cardiac MRI (CMR) findings confirming the presence of MI (arrows) in both cases based on late gadolinium-enhanced (LGE) CMR images with (arrows) and without IMH based on T2*CMR. Note that the [hs-cTn-I]threshold for detection of MI is 0.04734 ng/ml compared to the multi-fold change in [hs-cTn-I]post-PCI in both hemorrhagic and non-hemorrhagic STEMIs. However, note that the magnitude and pattern of [hs-cTn-I]elevations post-PCI in hemorrhagic MI is significantly different from non-hemorrhagic MI.

FIGS. 1D and 1E depict diagnostic performance. FIG. 1D shows the performance metrics of the discovery cohort with AUC nearly greater than 0.9 after the first hour of reperfusion upto 24 hours. FIG. 1E shows the Cohen's Kappa (x) values across various timepoints post-reperfusion in the validation cohort, offering a statistical measure of the consistency and reliability of hemorrhagic MI diagnosis compared to the CMR gold standard. x value ranges between 0.6 to 0.8 (shown by the shaded area) with peak at initial 2 hours post-reperfusion shows substantial agreement between gold-standard CMR and [hs-cTn-I]threshold based detection of hemorrhagic MI.

FIGS. 1F-1H depict hemorrhagic injury overrides washout paradigm. FIG. 1F: This panel displays troponin release patterns following PCI in patients with TIMI grade 0, indicating total occlusion prior to intervention, across varying infarct sizes. The data reveal divergent troponin kinetics between hemorrhagic and non-hemorrhagic MIs, underscoring that the presence of hemorrhage rather than the degree of occlusion or post-reperfusion washout hypothesis that primarily drives the distinct troponin kinetics observed. FIG. 1G: Troponin kinetics for myocardial infarctions with a matched infarct size (15-30% of left ventricular mass), irrespective of pre-PCI TIMI grade. This uniform infarct size range serves as a controlled variable to isolate the effect of IMH on troponin release. The results consistently demonstrate that infarct size alone does not account for the variability in troponin levels, emphasizing the impact of hemorrhagic transformation within the myocardium. FIG. 1H: This panel specifically correlates troponin kinetics of MIs with both total occlusion (TIMI 0) and a standardized myocardial infarct size (15-30% LV), during the post-reperfusion phase. It highlights that, within this controlled context, hemorrhagic myocardial infarctions exhibit markedly different troponin release patterns compared to non-hemorrhagic cases. This divergence in troponin kinetics provides compelling evidence that the pathophysiological basis of troponin elevation post-PCI is primarily driven by the presence of intramyocardial hemorrhage, rather than by the pre-reperfusion arterial patency driven washout hypothesis or the size of the myocardial infarct alone.

FIG. 2: Multi-modal Validation of Post-Reperfusion Troponin for Diagnosing Hemorrhagic MI, showing that troponin kinetics in hemorrhagic MI reflect cardiomyocyte injury driven by histopathological evidence of microarteriole rupture and RBC extravasation. Representative cases of hemorrhagic (panel A) and non-hemorrhagic (panel B) MIs with respective [hs-cTn-I] kinetics plotted on a semilogarithmic scale are shown with horizontal axis depicting the time after reperfusion. CMR images present raw and processed images of the intramyocardial hemorrhage area (T2*), infarct location and size by late gadolinium enhancement (LGE). In processed images intramyocardial hemorrhage is shown in red color; infarct region is shown in pale orange color; and microvascular obstruction are identified as gray regions within the infarction. Moreover, the figure includes an optical image of a mid-ventricular slice from the same canine, along with a trichome-stained section of the slice (demarcated on the optical image; scale bar: 10000 um). The selected remote and infarct zones are shown as compact normal and infarcted cardiomyocytes at 100 um scale, revealing extravascular interstitial red blood cells (RBCs) and ruptured arterioles in the infarct zone of the hemorrhagic MI case, but the absence of red blood cells s and intact arterioles in the non-hemorrhagic MI case.

FIG. 3 depict that heme-driven cardiomyocyte death dictates troponin kinetics and infarct expansion in hemorrhagic MI. Panel A demonstrates infarct size (% LV) at 0 and 24 hrs post-reperfusion in a canine model. Nonhemorrhagic MI shows a +10% change (n=7; p=0.16), while hemorrhagic MI reveals a substantial +45% expansion (n=14; p=0.0002), as measured by CMR-LGE. Panel B: Elevated HCP-1 Levels in Hemorrhagic Myocardial Infarction. The graph compares HCP-1 expression, showing a significant increase in the hMI group compared to controls as well as non-hMI (p=0.002 & 0.003 respectively), while Non-hMI levels are comparable to controls (p=0.26). Data are mean±SD from three subjects per group. Panel C: Presents iPSC-CM viability and corresponding hs-cTnI levels after exposure to increasing hemin concentrations. A dose-dependent decline in viability and rise in hs-cTnI confirm heme's detrimental impact in hMI. Abbreviations: % LV, percent of left ventricular mass; CMR-LGE, cardiac magnetic resonance with late gadolinium enhancement; iPSC-CM, induced pluripotent stem cell-derived cardiomyocytes; hs-cTnI, high-sensitivity cardiac troponin I; Hemin, a heme oxygenase substrate; HO-1, Heme oxygenase-1; HCP-1, Heme carrier protein-1; GAPDH Glyceraldehyde 3-Phosphate Dehydrogenase; hMI, Hemorrhagic MI; Non-hMI, Nonhemorrhagic MI.

FIGS. 4A-4B depict that hemorrhagic myocardial infarction is associated with adverse early clinical outcomes post-reperfusion. FIG. 4A: Multivariate Adj.-OR ratio estimates of, length of index hospitalization (RR 3.05) and in-hospital mortality (RR 2.78) following hemorrhagic STEMI. The multivariate regression encompasses co-morbidity factors including age, gender, hypertension, diabetes mellitus, dyslipidemia, smoking, and the use of medications, inclusive of Beta-Blockers, ACE inhibitors, ARBs, Statins and Aspirin prior to PCI. Medications during the catheterization procedure includes GPIIbIIIa inhibitors, anticoagulants (heparin, bivalirudin, warfarin and low molecular weight heparin) and antiplatelets (aspirin, clopidogrel, prasugrel, ticagrelor and cangrelor). FIG. 4B: 30-day outcomes table of reperfused STEMI patients (n=6180), divided into hemorrhagic (n=1323) and non-hemorrhagic (n=4857) STEMI groups. Hemorrhagic STEMI is associated with significantly elevated 30-day and in-hospital mortality (p<0.0001), a higher risk of 30-day readmission (p<0.0001), and increased incidence of acute heart failure (HF) (p<0.0001). Statistically significant differences were not observed in the occurrence of stable angina, unstable angina, re-infarction, arrhythmia, or stroke. However, arrhythmia and stroke showed higher odds in hemorrhagic STEMIs after multivariate matching. Acronyms: HTN hypertension, DM diabetes mellitus, DLP dyslipidemia, BB beta blocker, ACE (−) angiotensin converting enzyme inhibitor, ARB angiotensin receptor blocker, and GP Iib/IIIa (−) glycoprotein Iib/IIIa inhibitor. *Pre-existing medications before hospitalization. † Loading medications during PCI. P2Y12 inhibitors includes Clopidogrel, Prasugrel, Ticagrelor and Cangrelor. Anticoagulants include heparin, Low-molecular weight Heparin, Bivalirudin and Fondaparinux.

FIGS. 5A-5B depict that differential 30-Day survival and freedom from rehospitalization in reperfused STEMI shows prognostic significance of hemorrhagic MI. Kaplan-Meier survival curves depicting 30-day mortality rates in patients with hemorrhagic versus non-hemorrhagic STEMI are shown. Out of the total 6,180 reperfused STEMI patients, 264 (4.27%) experienced mortality within the first 30 days. Among the hemorrhagic STEMI cohort (n=1323), 113 (8.54%) did not survive the 30-day period. In contrast, among the non-hemorrhagic STEMI cohort (n=4857), 151 (3.11%) experienced 30-day mortality. The analysis began from day 0 (after PCI), revealing a statistically significant difference (p=0.0001) in survival rates between the two groups. FIG. 5A shows Kaplan-Meier survival curves for the 30-day mortality rates of a cohort of 6,180 patients, differentiated into hemorrhagic STEMI (n=1,323) and non-hemorrhagic STEMI (n=4,857) groups. 113 of the patients with hemorrhagic STEMI (8.54%) did not survive the initial 30 days, compared to 151 patients with non-hemorrhagic MI (3.11%). The survival curves reveal a highly significant difference (p=0.0001) in mortality rates between these two cohorts. FIG. 5B shows Kaplan-Meier survival curves for 30-day rehospitalization rates, with 138 out of 1,323 patients in the hemorrhagic STEMI cohort (10.44%) and 364 out of 4,857 patients in the non-hemorrhagic STEMI cohort (7.49%) requiring rehospitalization within 30 days (p<0.0001).

FIG. 6A: demonstration of Post-PCI Troponin based Diagnosis of Hemorrhagic STEMI, Patient Prognosis, and the Prevalence of Hemorrhagic STEMI in the United States.

FIG. 6B: Flow Chart Summarizing Patient Selection for MIRON-TROP. FIG. 6C: Flow Chart Summarizing Patient Selection for MIRON-ACUTE. FIG. 6D: Flow Chart Summarizing Patient Selection for MIRON-NATIONAL.

FIG. 7: Bar charts comparing time for [hs-cTn-I] to peak (left) and time for Rate [hs-cTn-I] to peak (right) in animals. In each chart, the x-axis represents the two MI types, while the y-axis indicates time in hours. The bars are color-coded, with red bars representing hemorrhagic MI and grey bars representing non-hemorrhagic MI. The right chart shows that hemorrhagic animals have a shorter time to reach peak concentration compared to non-hemorrhagic MI models, while the left chart demonstrates that hemorrhagic animals also exhibit a short time to achieve peak Rate [hs-cTn-I]. We performed controlled ischemia followed by reperfusion of the left-anterior-descending coronary artery in canines (n=25) to simulate a mechanically revascularized MI. Four animals died following reperfusion (two within 1 hour of reperfusion; and two within 3 days of reperfusion) and another two animals failed to develop MI. Thus, 19 animals were available for serial studies and were followed for 72 hours. On CMR performed at 3 days post reperfusion, persistent microvascular obstruction was evident in 17 animals. IMH was evident in 14 (74%; INM+); the other 5 animals were identified as INM−.

FIG. 8: A comprehensive evaluation of [hs-cTn-I] kinetics in canine subjects with controlled ischemia-reperfusion protocol resulting into hemorrhagic (n=5) and non-hemorrhagic MI (n=14) (panel A), accompanied by a correlation regression analysis that explains the relationship between these kinetics and intramyocardial hemorrhage volume (panel B), providing crucial insights into the pathophysiology and potential diagnostic implications. Aggregate [hs-cTn-I]levels across all animals at baseline, immediately after reperfusion, and at 1, 3, 7, 15, 24, 48, and 72 hours after reperfusion are shown in FIG. 5A. Prior to reperfusion (during ischemia), both hemorrhagic and non-hemorrhagic animals did not show any statistically significant difference in [hs-cTn-I](p=0.21). However, animals positive for hemorrhagic MI showed a very rapid rise in [hs-cTn-I] in the immediate post-reperfusion period (0-4 hours, p<0.05) and a rapid fall after 48 hours of reperfusion. In contrast, in the non-hemorrhagic group there was no difference in [hs-cTn-I]over the first 4 hours (p=0.12) but marked difference at 24 hours (p=0.003) post-reperfusion. Animals without hemorrhagic MI showed a gradual rise in [hs-cTn-I]during the initial 24 hours and gradual fall thereafter. The divergence of [hs-cTn-I] kinetics in initial 3 hours post-reperfusion is without ambiguity. Time for [hs-Trop-I] to peak in hemorrhagic animals was at 3.5±0.37 hours while [hs-cTn-I] of non-hemorrhagic animals peaked at 16.8±1.6 hours post reperfusion. Notably the peak [hs-cTn-I]was >3.5-fold higher (259.72±15.44 ng/ml (hemorrhagic) vs 72.95±10.66 ng/ml (non-hemorrhagic)) and >20 hours earlier in the hemorrhagic animals compared to the non-hemorrhagic counterparts.

FIG. 9: Aggregate Rate [hs-cTn-I]levels across all animals at baseline, immediately after reperfusion, and at 1, 3, 7, 15, 24, 48, and 72 hours after reperfusion. The difference in levels was normalized to hours in order to calculate troponin change rate as ng/ml/hr. Similar observations were made with Rate [hs-cTn-I] in canine with hemorrhagic MI as in patients with Rate [hs-cTn-I]peaking at approximately at the same time point following reperfusion. In animals without hemorrhagic MIs, a slow and low magnitude Rate [hs-cTn-I] with a weak peak was observed throughout 24 hours. Specifically peak Rate [hs-cTn-I]was >7-fold higher among hemorrhagic MI (66.28 ng/ml/hr) as compared to non-hemorrhagic cohort (8.86 ng/ml/hr). Peak Rate [hs-cTn-I]occurred at 1.71±0.25 hours in hemorrhagic MI compared to 6.6±2.1 hours in non-hemorrhagic MI subjects.

FIG. 10: Receiver operating characteristic (ROC) analysis and diagnostic performance of Rate [hs-cTn-I] in relation to IMH volume canine subjects. Panel A shows the correlation between IMH volume as determined from T2*CMR and Rate [hs-cTn-I]. Regression analysis of [hs-cTn-I] with hemorrhage volume showed highest correlation at 3 hours (R²=0.91, FIG. 8). Panel B shows the ROC curves for Rate [hs-cTn-I] to predict hemorrhage volume, with the area under the curve (AUC) OF 0.99 signifying the overall diagnostic accuracy.

FIG. 11: Relationship between post-reperfusion time and area-under-the-curve from receiver-operating-characteristic analysis of [hs-cTn-I]based detection of hemorrhage volume in patients and animals. The horizontal axis represents time in hours on a logarithmic scale, while the y-axis displays the correlation coefficient. A curve is plotted to depict the relationship between time and the correlation between [hs-cTn-I] and IMH volume on a logarithmic scale. The peak of the curve is observed at hour 3, indicating the highest correlation between [hs-cTn-I] and IMH volume at this time point. This finding suggests that the third hour may be a critical time for assessing the severity of IMH in myocardial infarction patients, with the logarithmic scale emphasizing the importance of early time points in the analysis.

FIG. 12: Relationship between post-reperfusion time and area-under-the-curve from receiver-operating-characteristic analysis of Rate [hs-cTn-I]based detection of hemorrhage volume in patients and animals. The x-axis represents time in hours displayed on a logarithmic scale, while the y-axis shows the AUC values. A curve is plotted to depict the association between time and the AUC of [hs-cTn-I]velocity and IMH volume. The peak of the curve is observed at hour 2, indicating the highest AUC and strongest association between [hs-cTn-I]velocity and IMH volume at this time point. This finding suggests that the first hour may be crucial for assessing the severity of IMH in myocardial infarction patients, with the logarithmic scale emphasizing the importance of early time points in the analysis.

FIG. 13: Temporal Evolution of [hs-cTn-I] Rate and its Clinical Relevance in Hemorrhagic and Non-Hemorrhagic MI Patients. Left panel shows Rate [hs-cTn-I] for patients with hemorrhagic (n=72) and non-hemorrhagic MI (n=79) over 48 hours. A striking divergence is observed in the first hour, with the rate of [hs-cTn-I]exceeding 10-fold higher in hemorrhagic MI patients compared to non-hemorrhagic MI patients. The right panels reveals a strong correlation, with an R-squared value of 0.7, emphasizing the substantial association between hemorrhage volume and Peak Rate [hs-cTn-I]rate.

FIG. 14: Diagnostic performance of [hs-cTn-I] and its Rate in differential diagnosis of hemorrhagic and non-hemorrhagic MI (n=151). A. Diagnostic Performance of [hs-cTn-I] in the Diagnosis of hMI. Panel A shows the diagnostic performance of [hs-cTn-I] for the identification of hemorrhagic MI. Using a cut-off value of 114.65 ng/ml, this diagnostic assay demonstrates a high sensitivity of 0.97, a specificity of 0.88, a positive predictive value (PPV) of 0.81, a negative predictive value (NPV) of 0.98, and an area under the curve (AUC) of 0.91. B. Diagnostic Performance of Rate [hs-cTn-I] in the diagnosis of hMI. Panel B presents the diagnostic performance of the rate of [hs-cTn-I] in the detection of hemorrhagic MI. Using a cut-off value of 52.36 ng/ml/hr, this diagnostic measure exhibits a sensitivity of 0.9333, a specificity of 0.8727, a positive predictive value (PPV) of 0.96, a negative predictive value (NPV) of 0.98, and an area under the curve (AUC) of 0.9624.

FIG. 15 depicts a summary of length of index hospitalization (by hours) between hemorrhagic and nonhemorrhagic MI patients in the MIRON-ACUTE study. The max number of days are 15 days for this analysis based on 99% of population. The three groups are based on 15 days of hospitalization grouped by initial >24 hrs, >48 hrs, >72 hrs. No one was discharged in <24 hrs (some patients did not survive): 98.96% of hemorrhagic MI patients stayed hospitalized for >24 hours (from when they received reperfusion therapy). In 48 hours many of the non-hemorrhagic patients gets discharged as well as a small percentage of hemorrhagic patients (who might have small hemorrhage): 86.31% of hemorrhagic MI patients stayed hospitalized for >48 hours. Within 72 hours majority of non-hemorrhagic MIs are discharged, however, a substantial number of hemorrhagic MI stayed hospitalized: (49.48% hMI patients stayed hospitalized for >72 hours).

FIG. 16 depicts the risk ratios of length of index hospitalization at >24 hours.

FIG. 17 depicts the risk ratios of length of index hospitalization at >48 hours.

FIG. 18 depicts the risk ratios of length of index hospitalization at >72 hours.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The terms “subject,” “patient,” or “individual” may be used interchangeably, and refer to a human or animal. In some embodiments, the subject is a human. In some embodiments, the subject is a vertebrate such as a primate, rodent, domestic animal or game animal. In an embodiment, the subject is mammal. The mammal can be a human, non-human primate, mouse, rat, dog, cat, horse, or cow, but are not limited to these examples. In addition, the methods described herein can be used to treat domesticated animals and/or pets.

A “subject in need” of diagnosis or treatment for a particular condition can be a subject suspected of having that condition, diagnosed as having that condition, already treated or being treated for that condition, not treated for that condition, or at risk of developing that condition. In some embodiments, the subject in need is a subject suspected of having hemorrhagic myocardial infarction, especially after a reperfusion therapy. In some embodiments, the subject in need is a subject with signs of myocardial infarction, such as chest pain, shortness of breath. In some embodiments, the subject in need is a subject experiencing or having undergone reperfusion following myocardial infarction. In some embodiments, the subject in need is a subject suffering from or having a high risk of having hemorrhage following intervention (e.g., reperfusion) after myocardial infarction. In some embodiments, the subject is a human patient with a myocardial infarction with total occlusion (TIMI 0). In some embodiments, the subject is a human patient with a myocardial infarction with any degree of occlusion. In some embodiments, the subject is a human patient with myocardial infarct size of 15-30% LV. In some embodiments, the subject is a human patient with myocardial infarct size of greater than 30% LV. In some embodiments, the subject is a human patient with myocardial infarct size of less than 15% LV.

A reperfusion therapy includes drugs and/or surgery. The drugs are thrombolytics and fibrinolytics used in a process called thrombolysis. Surgeries performed may be minimally-invasive endovascular procedures such as a percutaneous coronary intervention (PCI), which involves coronary angioplasty.

“Hemorrhage” as used herein refers to pooling of blood within a vessel or extravasation of blood into the interstitial space.

“Troponins” (or troponin) are a group of proteins found in skeletal and heart (cardiac) muscle fibers that regulate muscular contraction. Troponin tests measure the level of cardiac-specific troponin in the blood to help detect heart injury. There are three types of troponin proteins: troponin C, troponin T, and troponin I. In some embodiments, as shown in the Examples below, measuring a “cardiac troponin” level refers to measuring cardiac troponin I. In other embodiments, measuring a troponin level comprises measuring a level of troponin T, a level of troponin I, a level of troponin C, or a combination thereof.

The troponins (I, C, and T) are members of a complex of proteins that modulate the calcium-mediated interaction between actin and myosin within muscle cells. The nomenclature of these distinct proteins of the troponin complex is derived from their respective function in muscle contraction. Troponin T anchors the troponin complex to tropomyosin of the thin filament, whereas troponin I inhibits actomyosin ATPase, and troponin C is a calcium-binding subunit. Three isoforms of troponin I (TnI) have been identified: one associated with fast-twitch skeletal muscle, one with slow-twitch skeletal muscle, and one with cardiac muscle. The slow and fast-twitch isoforms have a similar molecular weight of approximately 20,000 dalton (Da) each. The cardiac-specific TnI isoform has a molecular weight of approximately 24,000 Da and contains a post-translational tail of 31 amino acids on the N-terminus of the molecule. This sequence and the 42% and 45% dissimilarity with the sequences of the other two isoforms have made possible the generation of highly specific monoclonal antibodies without cross-reactivity with other non-cardiac TnI forms. Therefore, unless otherwise stated, the level of troponin-I in various embodiments of the methods and systems disclosed herein refers to the level of cardiac troponin-I, including full protein or fragments, why may be detectable by monoclonal antibodies specifically directed against cardiac troponin-I (cTnI) and/or optical techniques such as with spectrophotometric sensors.

Herein we have demonstrated that high-sensitivity troponin-I is a cardiac-specific biomarker for cardiomyocyte injury and have demonstrated that it can be used in the diagnosis of intramyocardial hemorrhage both in heart attack patients and in large animal models with CMR as the ground truth. We have conceived and demonstrated that rapid, serial assessment of post-reperfusion, high-sensitivity troponin-I concentration ([hs-cTn-I]) can timely and accurately diagnose hemorrhagic MIs.

Various embodiments provide methods of examining/assaying a subject in need thereof, wherein optionally the subject has myocardial ischemia and has received a reperfusion therapy, and the method includes measuring a blood level of troponin-I in a biological sample obtained from the subject between immediately following the reperfusion therapy and within 48 hours following the reperfusion therapy, preferably between 0 and 20 hours, between 0 and 7.5 hours, more preferably between 0.5 and 5 hours after the reperfusion therapy. In some embodiments, the method is for a human subject, and the method includes measuring a blood level of troponin-I in a biological sample obtained from the subject between 0 and 3 hours (i.e., in the first, second, or third hour) following the reperfusion therapy. In some embodiments, the method is for a human subject, and the method includes measuring a blood level of troponin-I in a biological sample obtained from the subject in the first hour following the reperfusion therapy. In some embodiments, the method is for a human subject, and the method includes measuring a blood level of troponin-I in a biological sample obtained from the subject in the second hour following the reperfusion therapy. In some embodiments, the method is for a human subject, and the method includes measuring a blood level of troponin-I in a biological sample obtained from the subject in the third hour following the reperfusion therapy. In some embodiments, immediately following reperfusion therapy is within 1 minute following the reperfusion therapy. In some embodiments, immediately following reperfusion therapy is within 3 minutes following the reperfusion therapy. In some embodiments, immediately following reperfusion therapy is within 5 minutes following the reperfusion therapy. In some embodiments, immediately following reperfusion therapy is within 10 minutes following the reperfusion therapy.

Further embodiments provide that a subject (e.g., human subject) is indicated to have hemorrhagic myocardial infarction when the subject is detected with a high level of troponin-I in a blood sample obtained between immediately following a reperfusion therapy and 12 hours (e.g., between 0 and 0.5 hour, i.e., within the first 30 minutes; between 0 and 1 hour, i.e., in the first hour; between 1 and 2 hours, i.e., in the second hour; between 2 and 3 hours, i.e., within the third hour; or between 0 and 4 hours, i.e., in the first, second, third and/or fourth hour) following the reperfusion therapy, wherein the high level of the troponin-I is:
(1) (i) a level of troponin-I being at least 90 ng/mL in a blood sample obtained between 0.5 hour and 7.5 hours after the reperfusion therapy,
   (ii) a level of the troponin-I being at least 75 ng/mL in a blood sample obtained between 7.5 hours and 11.5 hours after the reperfusion therapy,
   (iii) a level of the troponin-I being at least 60 ng/mL in a blood sample obtained between 11.5 hours and 16.5 hours after the reperfusion therapy,
   (iv) a level of the troponin-I being at least 40 ng/mL in a blood sample obtained between 16.5 hours and 20.5 hours after the reperfusion therapy,
   (v) a level of the troponin-I being at least 30 ng/mL in a blood sample obtained between 20.5 hours and 24.5 hours after the reperfusion therapy, or
   (vi) a level of the troponin-I being at least 19 ng/mL in a blood sample obtained between 24.5 hours and 48.5 hours after the reperfusion therapy, or
   (vii) featuring any two, three, four, five, or all six of (i)-(vi);
(2) (viii) from a time series of the troponin-I level corresponding to blood samples obtained at two, three or more time points after the reperfusion therapy, there being a highest amount of the troponin-I level in a blood sample obtained between 3 hours and 5 hours following the reperfusion therapy, preferably at about 4 hours following the reperfusion therapy,
   (ix) from the time series of the troponin-I level, there being a highest amount of at least 300 ng/mL (or about 348±37 ng/mL), or
   (x) featuring both (viii) and (ix), i.e., the highest amount is at least 300 ng/mL (or about 348±37 ng/mL) and measured in the blood sample obtained between 3 hours and 5 hours (preferably at about 4 hours) following the reperfusion therapy;
(3) a concentration at least 7 times (e.g., 8 times, 9 times, 9 times, 10 times, 11 times, 12 times, 13 times, 14 times, 15 times or greater than), or more preferably at least 10 times, that obtained from a control subject (e.g., control human subject) not having hemorrhagic myocardial infarction, optionally wherein a concentration obtained from the control subject is obtained in a blood sample within 48 hours after reperfusion in the control subject, or between 9.5 and 11.5 hours after the reperfusion in the control subject; and/or
(4) featuring a high rate of increase in blood troponin-I level comparing a time point within first 2.5 hours after the reperfusion therapy (e.g., between 30 and 150 min, between 45 and 130 min, between 50 and 60 min, between 60 and 70 min, between 70 and 80 min, between 80 and 90 min, between 90 and 100 min, between 100 and 110 min, between 110 and 120 min, or between 120 and 130 min, after the reperfusion therapy) relative to one immediately after the reperfusion therapy, wherein the high rate of increase is at least 10 times (e.g., 11 times, 12 times, 13 times, 14 times, 15 times, 16 times, 17 times, 18 times, 19 times, 20 times, or greater than) that obtained from the control subject (e.g., control human subject).

Some embodiments provide that for a human subject, he/she is indicated to have hemorrhagic myocardial infarction when he/she is detected with a high level of the troponin-I in a biological sample obtained within the first 12 hours following a reperfusion therapy. For example, a human subject is indicated to have hemorrhagic myocardial infarction when detected with a high level of the troponin-I in in a blood sample obtained between 3 and 5 hours following the reperfusion therapy, i.e., in the fourth or fifth hour following the reperfusion therapy. In some embodiments, a high level of the troponin-I is at least 250 ng/mL. In some embodiments, a high level of the troponin-I is 250-300 ng/mL. In some embodiments, a high level of the troponin-I is 300-348 ng/mL. In some embodiments, a high level of the troponin-I is 348-360 ng/mL. In some embodiments, a high level of the troponin-I is 360-380 ng/mL. In some embodiments, a high level of the troponin-I is 380-400 ng/mL. In some embodiments, In some embodiments, a high level of the troponin-I is 400 ng/mL or greater. In some embodiments, a high level of the troponin-I is within 348.16±37.07 ng/mL. In some embodiments, a human subject is indicated to have hemorrhagic myocardial infarction when he/she is detected with a high rate of increase in blood troponin-I level of at least 100 ng/mL/hr. In some embodiments, a human subject is indicated to have hemorrhagic myocardial infarction when detected with a rate of increase in blood troponin-I level being 100-125 ng/mL/hr. In some embodiments, a human subject is indicated to have hemorrhagic myocardial infarction when detected with a rate of increase in blood troponin-I level being 125-150 ng/mL/hr. In some embodiments, a human subject is indicated to have hemorrhagic myocardial infarction when detected with a rate of increase in blood troponin-I level being 150-175 ng/mL/hr. In some embodiments, a human subject is indicated to have hemorrhagic myocardial infarction when detected with a rate of increase in blood troponin-I level being 175-200 ng/mL/hr. In some embodiments, a human subject is indicated to have hemorrhagic myocardial infarction when detected with a rate of increase in blood troponin-I level being 200-218 ng/mL/hr. In some embodiments, a human subject is indicated to have hemorrhagic myocardial infarction when detected with a rate of increase in blood troponin-I level being 218-230 ng/mL/hr. In some embodiments, a human subject is indicated to have hemorrhagic myocardial infarction when detected with a rate of increase in blood troponin-I level being 230-250 ng/mL/hr. In some embodiments, a human subject is indicated to have hemorrhagic myocardial infarction when detected with a rate of increase in blood troponin-I level being 250-270 ng/mL/hr. In some embodiments, a human subject is indicated to have hemorrhagic myocardial infarction when detected with a rate of increase in blood troponin-I level being 270-300 ng/mL/hr. In some embodiments, a human subject is indicated to have hemorrhagic myocardial infarction when detected with a rate of increase in blood troponin-I level being 300 ng/mL/hr or greater. In some embodiments, a human subject is indicated to have hemorrhagic myocardial infarction when detected with a rate of increase in blood troponin-I level being within 218.55±26.45 ng/mL/hr.

In other embodiments, the high rate of increase in the blood troponin-I level in a human subject indicated to have hemorrhagic myocardial infarction is the difference in the blood troponin-I levels between about 1 hour after the reperfusion therapy and immediately after the reperfusion therapy, divided by the time difference. In some embodiments, the high rate of increase in the blood troponin-I level in a human subject indicated to have hemorrhagic myocardial infarction is the difference in the blood troponin-I levels between about 0.5 hour after the reperfusion therapy and immediately after the reperfusion therapy, divided by the time difference. In some embodiments, the high rate of increase in the blood troponin-I level in a human subject indicated to have hemorrhagic myocardial infarction is the difference in the blood troponin-I levels between about 1.5 hour after the reperfusion therapy and immediately after the reperfusion therapy, divided by the time difference. In some embodiments, having said high rate of increase indicates the human subject has hemorrhagic myocardial infarction. In some embodiments, the high rate of increase in the blood troponin-I level in the human subject is at least 10 times that in a control human subject, i.e., at least 9-times higher than that in the control human subject, wherein the control human subject has received a reperfusion therapy following myocardial infarction but does not have reperfusion hemorrhage.

In some embodiments, the subject (e.g., human subject) indicated to have hemorrhagic myocardial infarction is further prescribed or administered with a treatment therapy to the subject. Preferably, the treatment therapy is effective to mitigate hemorrhagic myocardial injury, including but not limited to iron chelators and anti-inflammatory agents.

Some embodiments provide that the subject is a canine, and the canine subject is indicated to have hemorrhagic myocardial infarction when the canine subject is detected with a high level of the troponin-I concentration in a biological sample obtained between 0 and 6 hours following a reperfusion therapy. For example, the canine subject is indicated to have hemorrhagic myocardial infarction when the canine subject is detected with a high level of the troponin-I concentration in a biological sample obtained between 3 and 4 hours following the reperfusion therapy, i.e., in the fourth hour following the reperfusion therapy. As another example, the canine subject is indicated to have hemorrhagic myocardial infarction when the canine subject is detected with a high level of the troponin-I concentration in a biological sample obtained between 2 and 3 hours, i.e., in the third hour following the reperfusion therapy. In another instance, the canine subject is indicated to have hemorrhagic myocardial infarction when the canine subject is detected with a high level of the troponin-I concentration in a biological sample obtained or between 1 and 2 hours, i.e., in the second hour following the reperfusion therapy following a reperfusion therapy. In various instances, the high level of the troponin-I for a canine subject is at least 200 ng/mL. In some instances, the high level of the troponin-I for a canine subject is 200-259 ng/mL. In some instances, the high level of the troponin-I for a canine subject is 259-270 ng/mL. In some instances, the high level of the troponin-I for a canine subject is 270-300 ng/mL. In some instances, the high level of the troponin-I for a canine subject is 300-350 ng/mL. In some instances, the high level of the troponin-I for a canine subject is 350 ng/mL or greater. In some instances, the high level of the troponin-I for a canine subject is within 259.72±15.44 ng/mL.

In some embodiments, a canine subject is indicated to have hemorrhagic myocardial infarction if it has a high rate of increase in the blood troponin-I level following a reperfusion therapy to myocardial ischemia being at least 50 ng/mL/hr. In some embodiments, a canine subject is indicated to have hemorrhagic myocardial infarction if it has a high rate of increase in the blood troponin-I level following a reperfusion therapy to myocardial ischemia being 50-65 ng/mL/hr. In some embodiments, a canine subject is indicated to have hemorrhagic myocardial infarction if it has a high rate of increase in the blood troponin-I level following a reperfusion therapy to myocardial ischemia being 66-80 ng/mL/hr. In some embodiments, a canine subject is indicated to have hemorrhagic myocardial infarction if it has a high rate of increase in the blood troponin-I level following a reperfusion therapy to myocardial ischemia being 80-100 ng/mL/hr. In some embodiments, a canine subject is indicated to have hemorrhagic myocardial infarction if it has a high rate of increase in the blood troponin-I level following a reperfusion therapy to myocardial ischemia being 100-120 ng/mL/hr. In some embodiments, a canine subject is indicated to have hemorrhagic myocardial infarction if it has a high rate of increase in the blood troponin-I level following a reperfusion therapy to myocardial ischemia being 120 ng/mL/hr or greater. In some embodiments, a canine subject is indicated to have hemorrhagic myocardial infarction if it has a high rate of increase in the blood troponin-I level following a reperfusion therapy to myocardial ischemia being about 66 ng/mL/hr.

In further embodiments, the high rate of increase in the blood troponin-I level is comparing the amount in a sample obtained between 1.5 hours and 2 hours after the reperfusion therapy relative to one obtained immediately after the reperfusion therapy. In some embodiments, a canine subject is indicated to have hemorrhagic myocardial infarction if its high rate of increase in the blood troponin-I level is at least 10 times that in a control canine subject, optionally the rate of increase in said control canine subject is comparing a level obtained between 4 hours and 0 hours after the reperfusion therapy relative to a level obtained immediately following the reperfusion therapy. In some embodiments, the method further comprises prescribing or administering a treatment therapy to the canine subject.

A rate of increase is generally measured as the difference in the levels of two different times divided by the time gap between the two different times. In alternative embodiments, a rate of increase is a derivative of the levels over time if measurements of two or more time points are fitted into a mathematical formula. For example, in some embodiments, a rate of increase is an average rate, i.e., computed as the difference in the troponin concentration immediately after reperfusion relative to another later time point divided by the time lapse between the two time points. Mathematically, this can be expressed as: Rate [hs-cTn-I(t)]=(hs-cTn-I(t)-hs-cTn-I₀)/t, wherein ‘t’ denotes the time elapsed since reperfusion, ‘hs-cTn-I’ denotes high-sensitivity cardiac troponin-I concentration, ‘hs-cTn-I(t)’ denotes a high-sensitivity cardiac troponin-I concentration at time t, ‘hs-cTn-I₀’ denotes a high-sensitivity cardiac troponin-I concentration at baseline (prior to reperfusion or revascularization). For example, hs-cTn-I(0) is a first troponin sample right after PCI; and hs-cTn-I(1) is troponin sample at 1 hour after PCI. Hence various embodiments provide that in obtaining Rate [hs-cTn-I(t)], a blood sampling or noninvasive spectrometry-based sensor measurement may be performed at two or more time points, for example t₁, t₂, . . . ) at an interval of 1, 2, 3, 4, 5, 6, 7, 8, 9, 10, 15, 20, 30, 40, 50, or 60 minutes, preferably in the first 5 hours after the reperfusion, so as to calculate Rate [hs-cTn-I(t)]=(hs-cTn-I(t₂)-hs-cTn-I(t₁))/(t₂-t₁), preferably t₁ the pre-PCI baseline level (denoted as “nought”). In other embodiments, a rate compared to immediately after reperfusion may also be calculated, e.g., t₁ is within 3 minutes after the reperfusion.

Various embodiments provide methods of detecting the presence of hemorrhagic myocardial infarction after reperfusion therapy in a subject. In some embodiments, a human subject is indicated to have myocardial hemorrhage when he/she is detected with a high level of troponin-I in a blood sample obtained between 1 and 5 hours, that is in the second, third, fourth, or fifth hour, following the reperfusion therapy. In preferably embodiments, a human subject is detected with a high level of troponin-I in a blood sample obtained between 3 and 5 hours (that is in the fourth or fifth hour) following the reperfusion therapy. In various embodiments, the high level of the troponin-I in blood of a human subject is 300 ng/mL or greater. In some embodiments, the high level of the troponin-I in blood of a human subject is 250 ng/mL or greater. In some embodiments, the high level of the troponin-I in blood of a human subject is 250-300 ng/mL. In some embodiments, the high level of the troponin-I in blood of a human subject is 300-348 ng/mL. In some embodiments, the high level of the troponin-I in blood of a human subject is 348-360 ng/mL. In some embodiments, the high level of the troponin-I in blood of a human subject is 360-380 ng/mL. In some embodiments, the high level of the troponin-I in blood of a human subject is 380-400 ng/mL. In some embodiments, the high level of the troponin-I in blood of a human subject is 400 ng/mL or greater. In some embodiments, the high level of the troponin-I in blood of a human subject is within 348±37 ng/mL.

Some embodiments provide that a human subject is indicated to have myocardial hemorrhage when he/she is detected with a high rate of increase in troponin-I level, measured from a blood sample obtained between 0 and 2 hours. In some embodiments, a human subject is detected with a high rate of increase in the troponin-I level from a blood sample obtained between 0 and 1 hour, that is in the first hour, following the reperfusion therapy. In various instances, the high rate of increase in the blood troponin-I level in human is 100 ng/mL/hr or greater. In some example, a high rate of increase in the blood troponin-I level in human is 100-125 ng/mL/hr. In some example, a high rate of increase in the blood troponin-I level in human is 125-150 ng/mL/hr. In some example, a high rate of increase in the blood troponin-I level in human is 150-175 ng/mL/hr. In some example, a high rate of increase in the blood troponin-I level in human is 175-200 ng/mL/hr. In some example, a high rate of increase in the blood troponin-I level in human is 200-218 ng/mL/hr. In some example, a high rate of increase in the blood troponin-I level in human is 218-230 ng/mL/hr. In some example, a high rate of increase in the blood troponin-I level in human is 230-250 ng/mL/hr. In some example, a high rate of increase in the blood troponin-I level in human is 250-270 ng/mL/hr. In some example, a high rate of increase in the blood troponin-I level in human is 270-300 ng/mL/hr. In some example, a high rate of increase in the blood troponin-I level in human is greater than 300 ng/mL/hr. In some example, a high rate of increase in the blood troponin-I level in human is within 218.55±26.45 ng/mL/hr.

In some embodiments, a human subject is indicated to have myocardial hemorrhage when he/she is detected with a high level of troponin-I in a blood sample obtained between 0 and 6 hours after reperfusion therapy, wherein the high level of the troponin-I is a concentration at least 10 times that obtained from a control subject not having hemorrhagic myocardial infarction and/or the high level of the troponin-I has a rate of increase at least 10 times that obtained from the control subject.

Various embodiments provide methods of treating a subject in need thereof, wherein the subject has myocardial ischemia and received a reperfusion therapy, and the methods include administering a treatment therapy to the subject, wherein the subject is detected with a high level of troponin-I in a blood sample obtained between 0 and 12 hours following the reperfusion therapy, wherein the treatment therapy comprises an iron chelator, an agent that binds or regulates heme, and/or an anti-inflammatory agent. In some instances, a method for treating a subject includes administering a treatment therapy to a subjects detected with a high level of troponin-I in a blood sample obtained between 0 and 1.5 hours following a reperfusion therapy to myocardial ischemia. In some instances, a method for treating a subject includes administering a treatment therapy to a subjects detected with a high level of troponin-I in a blood sample obtained between 1.5 and 6 hours following a reperfusion therapy to myocardial ischemia. In some instances, a method for treating a subject includes administering a treatment therapy to a subjects detected with a high level of troponin-I in a blood sample obtained between 3 and 5 hours, i.e., the fourth or fifth hour, following a reperfusion therapy to myocardial ischemia. In various instances, a high level of the troponin-I is a concentration at least 10 times that obtained from a control subject not having hemorrhagic myocardial infarction. In some instances, a high level of the troponin-I is a concentration at least 8 times greater than that obtained from a control subject not having hemorrhagic myocardial infarction. In some instances, a high level of the troponin-I is a concentration at least 9 times greater than that obtained from a control subject not having hemorrhagic myocardial infarction. In some instances, a high level of the troponin-I is a concentration at least 10 times greater than that obtained from a control subject not having hemorrhagic myocardial infarction. In some instances, a high level of the troponin-I is a concentration at least 11 times greater than that obtained from a control subject not having hemorrhagic myocardial infarction. In some instances, a high level of the troponin-I is a concentration at least 12 times greater than that obtained from a control subject not having hemorrhagic myocardial infarction. In some instances, a high level of the troponin-I is a concentration at least 13 times greater than that obtained from a control subject not having hemorrhagic myocardial infarction. In some instances, a high level of the troponin-I is a concentration at least 14 times greater than that obtained from a control subject not having hemorrhagic myocardial infarction. In some instances, a high level of the troponin-I is a concentration at least 15 times greater than that obtained from a control subject not having hemorrhagic myocardial infarction. In some embodiments, a high level of the troponin-I has a high rate of increase at least 10 times that obtained from the control subject. In some embodiments, a high level of the troponin-I has a high rate of increase at least 11 times greater than that obtained from the control subject. In some embodiments, a high level of the troponin-I has a high rate of increase at least 12 times greater than that obtained from the control subject. In some embodiments, a high level of the troponin-I has a high rate of increase at least 13 times greater than that obtained from the control subject. In some embodiments, a high level of the troponin-I has a high rate of increase at least 14 times greater than that obtained from the control subject. In some embodiments, a high level of the troponin-I has a high rate of increase at least 15 times greater than that obtained from the control subject. In some embodiments, a high level of the troponin-I has a high rate of increase at least 16 times greater than that obtained from the control subject. In some embodiments, a high level of the troponin-I has a high rate of increase at least 17 times greater than that obtained from the control subject. In some embodiments, a high level of the troponin-I has a high rate of increase at least 18 times greater than that obtained from the control subject. In some embodiments, a high level of the troponin-I has a high rate of increase at least 19 times greater than that obtained from the control subject.

Some embodiments provide methods of treating a human subject in need thereof, wherein the human subject has myocardial ischemia and received a reperfusion therapy, the methods include administering a treatment therapy to the human subject, wherein the human subject is detected with a high level of troponin-I in a blood sample obtained between 1 and 5 hours, that is in the second, third, fourth, or fifth hour, following the reperfusion therapy, wherein the high level of the troponin-I is at least 300 ng/mL or 250 ng/mL (e.g., 250-300, 300-348, 348-360, 360-380, 380-400 ng/mL, or greater than 400 ng/mL) or within 348±37 ng/mL. Some embodiments provide that the troponin-I has a high rate of increase, measured from a blood sample obtained between 0 and 2 hours, more preferably between 0 and 1 hour, that is in the first hour following the reperfusion therapy, and the high rate of increase is at least 200 ng/mL/hr or 100 ng/mL/hr (e.g., 100-125, 125-150, 150-175, 175-200, 200-218, 218-230, 230-250, 250-270, 270-300 ng/mL/hr, or greater than 300 ng/mL/hr) or within 218.55±26.45 ng/mL/hr. In some embodiments, the treatment therapy comprises an iron chelator, an agent that binds or regulates heme, and/or an anti-inflammatory agent.

Some embodiments provide methods for treating a canine subject in need thereof, wherein the canine subject has myocardial ischemia and received a reperfusion therapy, the methods include administering a treatment therapy to the canine subject, wherein the canine subject is detected with a high level of troponin-I in a blood sample obtained between 1 and 4 hours, that is in the second, third, and fourth hours, following the reperfusion therapy, more preferably between 2 and 3 hours (that is within the third hour) following the reperfusion therapy, wherein the high level of the troponin-I is at least 200 ng/mL (e.g., 200-259, 259-270, 270-300, 300-350 ng/mL or greater than 350 ng/mL) or within 259±15 ng/mL. In some embodiments, the canine subject is detected with a high rate of increase in the troponin-I level, measured from a blood sample obtained between 0 and 2 hours, more preferably between 0 and 1 hour, that is in the first hour following the reperfusion therapy, and the high rate of increase is at least 60 ng/mL/hr or 50 ng/mL/hr (e.g., 50-65, 66-80, 80-100, 100-120 ng/mL/hr, or greater than 120 ng/mL/hr) or about 66 ng/mL/hr, and wherein the treatment therapy comprises an iron chelator, an agent that binds or regulates heme, and/or an anti-inflammatory agent.

In some embodiments, the troponin-I of the control subject is obtained from the control subject in a same timeframe following a reperfusion therapy to the control subject. In other embodiments, the troponin-I of the control subject is a highest amount obtained from the control subject in a time series following a reperfusion therapy to the control subject.

In various embodiments, the treatment therapy is given immediately after detecting the high level of the troponin-I in the subject, optionally being within 3 hours (i.e., from 0 to 3 hours) after the detection. In various embodiments, the treatment therapy comprises the iron chelator, and the iron chelator comprises a ferrous iron chelator, a ferric iron chelator, or both. Exemplary iron chelators include but are not limited to ferrous iron chelators (e.g., dexrazoxane, 2,2-bipyridl), and ferric iron chelators (desferrioxamine, deferiprone, deferasirox, hinokitiol, pyridoxal isonicotinoyl hydrazone, salicylaldehyde isonicotinoyl hydrazone).

In other embodiments, the treatment therapy comprises an agent that binds or regulates heme, including for example hinokitiol, hemopexin, a heme oxygenase-1, haptoglobin, albumin, ferritin, al-microglobulin, al-antitrypsin, glutathione-S-transferase, liver fatty acid binding protein, heme-binding protein 23 (also known as peroxiredoxin), p22 heme binding protein, and glyceraldehyde-3-phosphate dehydrogenase, nuclear factor E2 related factor 2 (Nrf2), feline leukemia virus subgroup C receptor 1a (FLVCR1a), FLVCR2, or ATP-binding cassette subfamily G member 2 (ABCG2). In some embodiments, the treatment therapy comprises a combination of two or more of an iron chelator, an agent that binds or regulates heme, and an anti-inflammatory agent.

Additional embodiments provide methods of monitoring a subject undergoing a reperfusion therapy for an ischemic heart disease and/or assessing efficacy of a treatment therapy against ischemia-reperfusion injury in the subject with the ischemic heart disease, and the methods include: measuring a level of troponin-I in a blood sample obtained between 0-20 hours following the reperfusion therapy from the subject; administering a treatment therapy to the subject when the subject is measured with a high level of the troponin-I in a blood sample obtained between 0 and 20 hours following the reperfusion therapy; and further measuring a level of the troponin-I in a blood sample obtained after the treatment therapy or after the previous measurement.

In some embodiments, a high level of the troponin-I in the blood sample obtained between 0-20 hours following the reperfusion therapy is a concentration at least 10 times that obtained from a control subject not having hemorrhagic myocardial infarction and/or the high level of the troponin-I has a rate of increase at least 10 times that obtained from the control subject.

In some embodiments, for a human, a high level of the troponin-I in the blood sample obtained between 0 and 6 hours following the reperfusion therapy is further at least 250 ng/mL or within 348±37 ng/mL or has a rate of increase of at least 190 ng/mL/hr or within 218±26 ng/mL/hr.

In some embodiments, for a canine, a high level of the troponin-I in the blood sample obtained between 0 and 6 hours following the reperfusion therapy is further at least 200 ng/mL or within 259±15 ng/mL or has a rate of increase of at least 60 ng/mL/hr or about 66 ng/mL/hr.

In some embodiments of the monitoring or assessing treatment therapy efficacy methods, the methods further include administering a second dose of the treatment therapy when the further measurement measures a higher level of the troponin-I relative to that obtained at a same time lag after a reperfusion therapy in a reference subject whose hemorrhagic myocardial infarct is maintained.

In various embodiments, a level of troponin in a blood sample or in a biological sample comprising blood is a concentration of the troponin in serum or plasma. In some embodiments, a biological sample is serum. In some embodiments, the biological sample is plasma. In some embodiments, the biological sample is a fresh plasma. In other embodiments, the biological sample is a frozen plasma. In some embodiments, the biological sample is lithium heparin plasma. In some embodiments, lithium heparin plasma and serum samples are not used interchangeably in a same measurement assay. In some embodiments, a fresh sample is immediately measured in a quantitative assay for the amount of troponin-I level. In other embodiments, a sample obtained from a subject is frozen and stored up to 180 days before quantitative measurement.

Techniques to measure a blood level of troponin include enzyme-linked immunosorbent assays. Kits for assaying troponin are available including ACCESS hsTnI (High Sensitivity Troponin I Assay) immunoassay systems, Beckman Coulter; cardiac troponin-I Immunoassays (Siemens Healthineers); or canine ultra-sensitive cardiac troponin-I ELISA kits from Life Diagnostics. For example, in measurement of human cardiac troponin-I (cTnI) level, monoclonal antibodies specifically directed against human cTnI are used, such as those provided in ACCESS hsTnI immunoassay systems by Beckman Coulter.

Other ways to measure troponin-I level from a subject may be performed non-invasively, such as optically through spectrophotometric sensors.

Further embodiments provide that the troponin levels disclosed herein are also for use in providing survival outcome prognosis of 30 days following reperfusion. In various embodiments, a subject indicated to have hemorrhagic myocardial infarction following the reperfusion therapy would have a poor prognosis of survival at 30 days and/or higher likelihood of requiring rehospitalization within 30 days following the reperfusion therapy; and vice versa.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

We performed multiparametric study of high-sensitivity troponin-I kinetics in an observational clinical trial in patients with reperfused MI (n=92). For histopathological validation of our findings, we performed post-reperfusion troponin kinetics in ischemia-reperfusion canine models (n=19). Blood specimens were collected at ischemia before PCI, and after PCI at every hour for 12 hours, and at 16, 20, 24, 48 hours post-reperfusion hours. To isolate the troponin kinetics of subjects with intramyocardial hemorrhage, all reperfused subjects were classified by time-lapse imaging with cardiovascular magnetic resonance as hemorrhagic or non-hemorrhagic.

We demonstrated that post-reperfusion troponin kinetics between hemorrhagic and non-hemorrhagic MI are completely very different. Non-hemorrhagic MI is characterized by slow rise and fall of troponin levels while the hemorrhagic MI is characterized by rapid rise and gradual fall of troponin levels in early post-reperfusion period. Hemorrhagic MI subjects have greater than 7-fold average increase in peak troponin concentration (human patients 348.16±264.74 ng/mL; canine 259.70±57.78 ng/mL) as compared to non-hemorrhagic MI subjects (human patients 30.95±29.67 ng/mL; canine 72.95±23.84 ng/mL). (A greater than 7-fold average increase is calculated as peak troponin in hemorrhagic MI/peak troponin in non-hemorrhagic MI.) We observed peak troponin concentration at 2-3 hours post-reperfusion. Moreover, hemorrhagic MI subjects showed greater than 10-fold average rate of change in troponin rise (human patients 218.55±188.91 ng/mL/hr; canine 66.29±16.05 ng/mL/hr) as compared to non-hemorrhagic MI subjects (human patients 9.09±14.28 ng/mL/hr; canine 8.87±6.30 ng/mL/hr). (A greater than 10-fold average rate of change in troponin rise can be calculated as peak average rate of change in troponin in hemorrhagic MI/peak average rate of change in troponin in non-hemorrhagic MI. The average rate of change is computed between right after PCI (e.g., within the first 5 minutes) and a later time point.) Peak troponin level was strongly correlated to intramyocardial hemorrhage volume as determined by CMR (R², human patients=0.82, canine=0.84).

Early identification of intramyocardial hemorrhage in acute myocardial infarction is critical from diagnostics and prognostics view. Our findings support the notion that post-reperfusion high-sensitivity troponin-I kinetics can be used as a clinical diagnostic biomarker and a highly sensitive method for time-sensitive diagnosis of IMH-driven ischemia-reperfusion injury.

Acute myocardial infarction (MI) is the leading public health issue in terms of cardiovascular disease burden, annual mortality, and life expectancy in the United States [1]. Emergency reperfusion therapy by percutaneous coronary intervention (PCI) is the standard of care with proven in-hospital mortality reductions in acute MI, but this has not translated into reduction in long-term cardiac mortality. In contrast, as evidenced by the HORIZON-AMI trial, immediate benefits of PCI with respect to myocardial salvage and improvement in ejection fraction post-revascularization are not always sustained. Notably, post PCI risk of congestive heart failure (CHF) two years following revascularization for STEMI is twice that of the risk prior to index STEMI [3], despite marked improvements in clinical acumen, ischemia diagnostics and reperfusion strategies over the past four decades.

Reperfusion therapy is not problem free; it may expand the zone of injury even after epicardial coronary artery patency is established. he recent CCS classification of acute MI based on tissue injury identifies hemorrhagic transformation of the MI zone as the most severe form of myocardial injury (stage IV) associated with reperfusion therapy. Solely based on cardiac MRI (CMR), it is estimated that on average hemorrhage is evident in nearly 40% of all successfully revascularized MI patients [5]; and that hemorrhage accelerates MI expansion, compromising myocardial salvage within the first 24 hours of revascularization [6]. Hemorrhagic MIs are also known to drive adverse outcomes in the chronic phase of MI and are casually linked to congestive heart failure. However, a key challenge with the identification of hemorrhagic MI is the requirement for CMR. CMR (although expanding) remains limited and is not recommended for use until days after PCI due to patient co-operation, and ongoing arrhythmias. An important consequence of this limitation is evidenced by the absence of knowledge whether hemorrhagic MI is centrally implicated in acute clinical outcomes. A viable alternative that is easily accessible, highly sensitive, cost-effective and broadly interpretable by all practitioners that identifies hemorrhagic MI patients across medical centers with or without CMR can broadly enable the determination of patient prognosis and develop new therapies.

Cardiac troponin concentration in blood, post PCI, has been shown to be significantly higher in hemorrhagic than in non-hemorrhagic acute MI patients [6]. However, it is unclear whether cardiac troponin can be used/developed into a robust diagnostic blood marker for identification of hemorrhagic MI, because it is not known (a) when the cardiac troponin peaks in hemorrhagic vs non-hemorrhagic MI; (b) whether the peak concentration of troponin is related to the extent of hemorrhage; (c) what cut-off values of cardiac troponin are indicative of hemorrhagic versus non-hemorrhagic MI; and (d) how quickly (or later) after PCI, hemorrhagic MI can be diagnosed. We hypothesized that rapid, serial assessments of high-sensitivity cardiac troponin I concentration ([hs-cTn-I]) levels kinetics allows for timely and accurate diagnosis of hemorrhagic MIs. We investigated this in revascularized MI patients with CMR-based diagnosis of IMH. This was followed by a comprehensive validation study in canine model of reperfused MI with CMR and post-sacrifice histology. Our findings open a fundamentally new paradigm to diagnose hemorrhagic MIs, enabling opportunities to improve patient management and development of therapies that mitigate IMH-mediated reperfusion injury.

### Clinical Trial on Post-Reperfusion Serum Troponin Kinetics

The study, approved by our Institutional Review Board and registered in clinicaltrials.gov (NCT05872308), prospectively enrolled consecutive STEMI patients undergoing primary PCI enrolled between June 2022 and May 2023. All patients provided written informed consent. Patients with hemodynamic instability or contraindications for CMR were excluded. Blood samples were obtained from the recruited patients (n=120) upon arrival at ER (0 hrs) (which is before PCI), immediately post-reperfusion (~1 hr since arrival at ER), and then every hour up to 12 hours (e.g, at 1, 2, 3, 4, 5, 6, 7, 8, 9, 10, 11, or 12 hours after reperfusion), and then at 16-, 20-, 24-, and 48 hours post-PCI. High-sensitivity cardiac troponin-I concentrations ([hs-cTn-I]) were assayed (ACCESS immunoassay systems, Beckman Coulter, California, USA). Cardiac magnetic resonance imaging (CMR) was performed post-PCI on a clinical CMR system (MAGNETOM Sola, Siemens Healthineers). CMR exam included cardiac cine (for function), multiple gradient-recalled echo (mGRE; for IMH diagnosis), and late gadolinium enhancement (LGE) (for infarct sizing) covering the entire left ventricle acquired with matched spatial resolutions (1.5×1.5×8 mm³) using standard fixed CMR protocols at 48-72 hours post-PCI.

### Preclinical Study Animal Preparation, Imaging, and Histology

A preclinical canine study was conducted on 25 canines (20-25 kg) to model the patient studies. Reperfused left-anterior-descending MIs were created. Blood samples were collected at multiple time points (during ischemia (noted as −1 hr), immediately after reperfusion (0 hr), and at 1, 3, 7, 15, 24, 48, 72 hrs post-reperfusion) for [hs-cTn-I]analysis.

### Techniques: Preclinical Study Animal Preparation

A preclinical canine study (n=25, 20-25 kg) was performed as approved by Institutional Animal and Usage Committee. Following left thoracotomy, no flow ischemia was created by occluding the left anterior descending coronary artery for 3 hours and then reperfused. Blood samples were collected at multiple time points (during ischemia (noted as −1 hr), immediately after reperfusion (0 hr), and at 1,3,7,15,24,48,72 hrs post reperfusion). [hs-cTn-I]was determined using two-site immunoassay method (ACCESS immunoassay systems, Beckman Coulter, California, USA). CMR was performed on a clinical system (Biograph mMR, Siemens Healthineers, Germany) 3 days post-reperfusion, with fixed 1.5×1.5×8 mm3 image resolution and imaging parameters including short-axis cine, multi-gradient echo (mGRE), and late gadolinium enhancement (LGE). LGE images identified MI and remote territories, with regions-of-interest used to determine mean T2*estimates of MI territories. IMH volume was calculated relative to total LV myocardial volume using mean-2SD threshold cut-off. Animals were subsequently humanely euthanized.

### Histology

A cohort of animals underwent histological evaluation as indicated by CMR (3 for IMH+ and 3 for IMH−). Heart tissue samples from canines were collected, fixed in buffered formalin, and processed for paraffin embedding. The tissues underwent re-fixation, dehydration, clearing, and paraffin embedding before being cast into sectioning blocks. Sections, approximately 5 m thick, were cut from paraffin-embedded heart tissues using a Leica RM2255 microtome (Leica Biosystems, Wetzlar, Germany). Masson's trichrome-stained paraffin sections were scanned and photographed using a Keyence BZ-X800 microscope (Keyence Corporation of America, USA).

### Statistical Analysis

Statistical analysis was performed using IBM SPSS Statistics 23 (IBM Corp., Armonk, New York). Normality of continuous data was determined by using the Shapiro-Wilk test and quantile-quantile plots. Continuous variables were expressed as mean±standard error. Categorical variables are reported as numbers and percentages. Differences between hemorrhagic and non-hemorrhagic MI groups were compared using Student's t-test. The chi-square test was used to compare differences in categorical variables. [hs-cTn-I] and Rate [hs-cTn-I], across different time points were compared using 2-way repeated measures ANOVA. Continuous variables were correlated using Pearson's correlation coefficient. ROC analysis was used to compute the area-under-the-curve (AUC) taking CMR-diagnosis as the ground truth. All tests were 2-tailed, and a P value<0.05 was indicative of statistical significance.

### Serum Troponin and CMR Analysis

T2*CMR was employed as the reference ground truth to retrospectively categorize reperfused MIs into hemorrhagic (hMI) and non-hemorrhagic (Non-hMI) groups, and to quantify the volume of IMH (% LV). MIs with an INM volume<4%, as determined by T2*CMR, were classified as non-hemorrhagic. The temporal kinetics of [hs-cTn-I] in hMI and Non-hMI subjects were assessed using the time series of hs-cTn-I concentrations. Concurrently, the rate of change in [hs-cTnI]relative to time (t) after reperfusion in hMI and Non-hMI was computed as

Rate [hs-cTn-I]=([hs-cTn-I(t)]-[hs-cTn-I₀])/t, where [hs-cTn-I(t)] and [hs-cTn-I₀] are [hs-cTn-I] at time t after reperfusion and baseline level before reperfusion or other revascularization techniques (e.g., immediately before reperfusion), respectively. Linear regression analysis was carried out to ascertain the relationships between IMH (% LV) and peak [hs-cTn-I], as well as Rate [hs-cTn-I]. Receiver operating characteristic (ROC) analysis was conducted to establish the cut-off values and diagnostic accuracy of hs-cTn-I and Rate [hs-cTn-I] for detecting hMI at various time points after reperfusion.

Results are shown in the figure set and described in the Brief Description of the Figures.

### Clinical Findings in Acute MI Patients Revascularized for STEMI

A total of 130 patients mechanically revascularized with primary PCI for STEMI were prospectively enrolled in the study. Logistical constraints resulting in challenges associated with accurate and timely blood sampling data from 9% (n=10) were not included. Accordingly, data from 120 patients were available for analysis. Based on CMR, participants were retrospectively classified into two categories, those with hemorrhagic MI (n=51) and those with non-hemorrhagic MI (n=69). Baseline patient characteristics along with CMR-measured parameters are shown in Table 1.

Hemorrhagic Transformation of Acute MI Leads to a Rapid Surge of Cardiac Troponin in Blood within Hours of Reperfusion.

Representative revascularized hemorrhagic and non-hemorrhagic STEMI patients identified with CMR, along with pre-PCI angiograms delineating the culprit coronary branches and corresponding highly time-resolved [hs-cTn-I] kinetics was evaluated. The hemorrhagic patient showed a sharp increase in [hs-cTn-I] within the 1-2 hours of reperfusion, compared to the non-hemorrhagic patient in whom the [hs-cTn-I]peak was gradual and peaking approximately 12 hours after. Aggregate (referring to average data of) [hs-cTn-I] and Rate [hs-cTn-I]levels across all patients prior to PCI, immediately after reperfusion, and the hours after up to two days are plotted. Prior to reperfusion (during ischemia), both parameters were not different in hemorrhagic and non-hemorrhagic patients (p>0.2). In the hemorrhagic MI group, [hs-cTn-I]peaked 6.5 hours earlier and had a magnitude that was 11-fold greater (4.02±0.78 hours with a peak of 348.16±37.07 ng/ml; herein data=average±SEM) than non-hemorrhagic MI (10.52±0.67 hours with a peak of 30.95±3.57 ng/ml; herein data=average±SEM). Further, the hemorrhagic MI group demonstrated a 20-fold higher average peak with respect to Rate [hs-cTn-I] of 218.55±26.45 ng/ml/hr in comparison to the non-hemorrhagic MI group (10.87±2.11 ng/ml/hr).

PEAK [Hs-cTn-I] AND PEAK RATE [Hs-cTn-I] Correlate Strongly with Hemorrhage Volume Measured with CMR.

The correlation between IMH volume as determined from T2*CMR and peak [hs-cTn-I] and Rate [hs-cTn-I] were evaluated. Regression analysis of [hs-cTn-I] with hemorrhage volume post reperfusion shows that highest correlation is achieved at 2-4 hours (R²=0.82, for [hs-cTn-I]; and R²=0.70, for Rate [hs-cTn-I]).

### Diagnostic Performance of Cardiac Troponin-Based Detection of Hemorrhagic MI is Robust Over the First 20 Hours Post Reperfusion and Diminishes Mildly Up to 48 Hours.

The sensitivity and specificity of [hs-cTn-I] for detecting hemorrhagic MI are greater than 80% over the first 20 hours post PCI. AUC associated with [hs-cTn-I]based detection of hemorrhagic MI, an indicator of the ability of the method to discriminate between hemorrhage and non-hemorrhagic MI patients based on cardiac troponin, was excellent (>0.9) for nearly up to 24 hours post PCI and decreasing only slightly thereafter by 48 hours PCI. However, the Rate [hs-cTn-I]was not as robust as [hs-cTn-I]after the first 5 hours PCI, indicating that while an early detection of hemorrhagic MIs can be identified accurately with [hs-cTn-I] and Rate [hs-cTn-I], delayed detection of hemorrhagic MI is best determined with [hs-cTn-I].

Hemorrhagic MI identified based on cardiac troponin kinetics shows microvascular rupture and extravasation of red blood cells into the interstitium. On histopathological examination of the infarct area, significant disruption of basement membrane and extravasation of RBCs were evident in hemorrhagic MI but in non-hemorrhagic MI. Red blood cells were dispersed in the interstitial space amongst infarcted cardiomyocytes in hemorrhagic MI but were absent in the infarct area of non-hemorrhagic MI. These histological findings validate the presence of acute microvascular rupture associated with intramyocardial hemorrhage as the cornerstone tissue injury in hemorrhagic MI, supporting findings on T2*CMR. These observations demonstrate the relationships between troponin kinetics, CMR and histology enabling the necessary validation for discriminating between hemorrhagic and non-hemorrhagic MIs based on cardiac troponin kinetics.

### Acute Clinical Outcomes:

In-hospital mortality: the presence of hMI was strongly associated with an increased odds of mortality during index hospitalization, with an odds ratio of 2.795 (95% CI: 2.084-3.748, p<0.0001, AUC 0.7513). Age, when analyzed in terms of days, demonstrated a significant impact on in-hospital mortality, with each additional day of age contributing to a 1.024 times higher odds of mortality (95% CI: 1.012-1.036, p=0.012). Conversely, gender showed no significant effect on mortality (OR: 1.109, 95% CI: 0.813-1.512, p=0.296). Patients with hypertension had 1.521 times higher odds of mortality (95% CI: 1.03-2.246, p=0.491), and those with diabetes had 1.714 times higher odds (95% CI: 1.257-2.336, p=0.457). Dyslipidemia had a protective effect on mortality, with an odds ratio of 0.669 (95% CI: 0.482-0.928, p=0.187). Smoking status (OR: 0.883, 95% CI: 0.683-1.143, p=0.2) and the use of beta-blockers (BB, OR: 1.687, 95% CI: 1.025-2.777, p=0.662) showed no significant associations with in-hospital mortality. Notably, the use of aspirin and P2Y12 inhibitors during PCI procedures had a protective effect on mortality (Aspirin OR: 0.343, 95% CI: 0.21-0.562, p<0.0001; P2Y12 OR: 0.35, 95% CI: 0.245-0.499, p<0.0001). In contrast, ACE inhibitors/ARBs, statins, GP IIb/IIIa inhibitors, and aspirin given during PCI procedures did not significantly impact mortality. These findings emphasize the critical importance of hMI as predictor of in-hospital mortality, along with the protective effects of specific medications during PCI procedures. Clinically, this information aids in risk assessment and treatment strategies for better in-patient management. 30-day mortality: hMI emerged as a significant risk factor, with an odds ratio of 2.85 (95% CI: 2.199-3.693, p<0.0001, AUC 0.743), the heightened vulnerability of hMI patients to adverse outcomes within the crucial 30-day period. Age, expressed in terms of days, displayed a significant impact, with each additional day associated with a 0.068% increase in the odds of 30-day mortality (p=0.022). Conversely, gender, hypertension, diabetes, dyslipidemia, smoking status, and beta-blockers showed no significant associations with 30-day mortality, suggesting their limited role as independent predictors. Remarkably, the use of aspirin and P2Y12 inhibitors during PCI procedures demonstrated a protective effect with significant p-values (Aspirin: p=0.463, P2Y12: p=0.536), underscoring the clinical importance of these medications in improving 30-day mortality outcomes. Conversely, post-medications, such as beta-blockers, ACE inhibitors/ARBs, statins, aspirin, P2Y12 inhibitors, and anticoagulants, did not significantly impact 30-day mortality. 30-day rehospitalization: Notably, the presence of hMI was associated with an increased odd of readmission, with an statistically significant risk ratio of 1.614 (95% CI: 1.283-2.032, p=0.0365, AUC 0.6253). Age, when analyzed in terms of days, had a marginal impact on readmissions, with a 1.008 times higher odds for each additional day of age (95% CI: 1.000-1.016, p=0.008). Gender demonstrated no significant effect (OR: 1.152, 95% CI: 0.934-1.422, p=0.218). Patients with hypertension and diabetes had odds ratios of 1.233 (95% CI: 0.95-1.6, p=0.283) and 1.023 (95% CI: 0.824-1.271, p=0.199) for readmission, respectively. Dyslipidemia (DLP), smoking, and the use of medications, including beta-blockers, ACE inhibitors/ARBs, statins, aspirin, and P2Y12 inhibitors, demonstrated varying influences on readmission rates. 30-day acute heart failure (HF): Presence of hMI is linked to a substantially higher risk of experiencing acute HF within 30 days, with an odds ratio of 2.255 (95% CI: 1.349-3.768, p=0.0019, AUC 0.6841). This implies that patients with Hmi face a markedly elevated risk of readmission due to acute HF. Moreover, age, when analyzed in terms of days, has a notable impact, with 1.033 times higher odds of acute HF for each additional day of age (95% CI: 1.012-1.054, p=0.0021). Conversely, gender does not exhibit a significant effect (OR: 0.7, 95% CI: 0.407-1.202, p=0.1961). Additionally, patients with hypertension have an odds ratio of 1.091 (95% CI: 0.95-2.183, p=0.546), while those with diabetes have an odds ratio of 0.98 (95% CI: 0.824-1.271, p=0.411) for readmission due to acute HF. The influence of other factors, including dyslipidemia, smoking, and medication usage (beta-blockers, ACE inhibitors/ARBs, statins, aspirin, and P2Y12 inhibitors), on acute HF rates is variable. 30-day arrhythmia events: The presence of Hmi exhibited a non-significant trend, with anan increased odds ratio (OR) of 1.333 (95% CI: 0.604-2.942, p=0.4767, AUC 0.7307), suggesting a potential association that didn't reach statistical significance. Age remained a marginal contributor, with each additional day increasing the odds by 2.8% (OR: 1.028, 95% CI: 0.996-1.060, p=0.032), indicating a minor impact of age on arrhythmia-related readmissions. Other factors, including gender, hypertension and diabetes were not found to have significant effects. The role of medications and comorbidities, such as dyslipidemia, smoking, and the use of drugs like beta-blockers, ACE inhibitors/ARBs, statins, aspirin, and P2Y12 inhibitors, demonstrated varying impacts on arrhythmia-related readmissions. These findings underscore the multifactorial nature of this outcome, emphasizing the need for tailored care and in-depth risk assessment to minimize arrhythmia-related readmissions and enhance patient well-being. It's important to note that some subgroups like aspirin and anticoagulants were excluded from the analysis due to small sample sizes.

### Additional 30-Day Outcomes:

Stable Angina: Patients with a history of hMI displayed a remarkable 41.9% lower risk of 30-day readmission (OR 0.581, 95% CI 0.35-0.966), while each year of age conferred a protective 2% reduction in angina risk (OR 0.964, 95% CI 0.996-0.016). Male gender exhibited a non-significant 19.7% increase in angina risk (OR 1.197, 95% CI 0.767-1.869). HTN was associated with a modest 32.4% reduction in angina risk (OR 0.676, 95% CI 1.195-0.294), whereas diabetes showed no substantial impact (OR 1.1, 95% CI 1.738-0.404). Dyslipidemia was linked to a 29.9% increase in the likelihood of angina (OR 1.294, 95% CI 2.154-0.86), and a smoking history significantly increased angina risk by 51.4% (OR 1.514, 95% CI 2.684-0.66).

In the context of pre-existing medications, ACE (−)/ARB demonstrated a marked reduction in angina risk (OR 2.997, 95% CI 8.221-1.905), while beta-blockers conveyed a 68.9% risk reduction (OR −1.1665, 95% CI 0.958-0.21). Notably, aspirin and P2Y12 inhibitors exhibited no significant impact on angina risk, with odds ratios of −1.2494 (95% CI 0.824-0.187) and −0.1376 (95% CI 1.629-0.405), respectively.

Unstable Angina: In this comprehensive study of 6,180 patients, we examined the risk factors associated with unstable angina (UA) readmission within 30 days, with a specific focus on the impact of hMI, co-morbidities and medications. Our analysis revealed that patients with unstable angina who had a history of hMI were significantly less likely to be readmitted for UA within 30 days (OR 0.575, 95% CI 0.37-0.893). Additionally, increasing age was inversely associated with UA readmission risk (OR 0.97, 95% CI 0.956-0.985). Male gender was found to be a predictor of UA (OR 1.498, 95% CI 0.332-0.498), while a history of HTN showed a modest protective effect (OR 0.413, 95% CI 1.164-0.281). Diabetes displayed a non-significant impact on readmission UA risk (OR 0.733, 95% CI 1.666-0.372). Dyslipidemia showed a trend towards increased readmission UA risk (OR 0.981, 95% CI 2.441-0.566), while smoking history was not a statistically significant predictor (OR 0.86, 95% CI 2.139-0.496). Among pre-existing medications, the use of ACE (−)/ARB was significantly associated with a reduced risk of readmission UA (OR 0.4341, 95% CI 5.089-1.245), while beta-blockers showed a protective trend (OR 0.4294, 95% CI 1.069-0.262). Conversely, the use of antiplatelet agents, such as aspirin (OR 0.4412, 95% CI 1.229-0.3) and P2Y12 inhibitors (OR 0.2905, 95% CI 1.218-0.299), did not significantly impact readmission UA risk. Notably, interventional therapies, including GP IIb/IIIa receptor inhibitors, aspirin (given during percutaneous coronary intervention), and certain discharge medications such as beta-blockers and statins displayed mixed associations with UA readmission risk.

Re-infarction: patients with hMI did not exhibit a significantly different risk of reinfarction compared to those without hMI, with an odds ratio (OR) of 0.795 (95% CI 0.435-1.452). While increasing age demonstrated a subtle protective trend, with a 2% risk reduction per year (OR 0.976, 95% CI 0.957-0.996), gender, hypertension and diabetes did not show substantial impacts. Dyslipidemia was associated with a 55.2% increase in reinfarction risk (OR 1.552, 95% CI 2.891-0.719), and a history of smoking displayed a 10.1% elevated risk (OR 1.101, 95% CI 1.935-0.475).

Regarding pre-existing medications, beta-blockers exhibited a 5.6% lower risk of reinfarction (OR 0.944, 95% CI 2.626-0.605), while aspirin and P2Y12 inhibitors displayed no substantial impact. The study further explored medications administered PCI procedures, which yielded variable associations with reinfarction risk.

CVA-Stroke: hMI did not significantly impact the risk of CVA, as the odds ratio (OR) was 0.921 (95% CI 0.279-3.047), indicating no substantial difference in CVA risk between the two groups. However, it's crucial to acknowledge the study's limitation, namely the relatively low number of CVA cases, with only 14 occurrences, making it challenging to draw robust conclusions.

The number of days since admission did not exhibit a substantial influence on CVA risk, with an OR of 1.011 (95% CI 0.97-1.054) per day, emphasizing the stability of CVA risk over time. Gender, hypertension, diabetes, dyslipidemia and smoking history did not demonstrate substantial effects on CVA risk, with ORs of 0.447 (95% CI 0.118-1.694), 1.139 (95% CI 0.309-4.208), 0.634 (95% CI 0.166-2.425), 0.41 (95% CI 0.126-1.339), and 1.404 (95% CI 0.714-2.76), respectively.

### Findings in a Canine Model of Reperfused MI

Canines (n=25) underwent controlled ischemia followed by reperfusion of the left-anterior-descending (LAD) coronary artery to simulate a mechanically revascularized MI. 4 animals died following reperfusion (2 within 1 hour of reperfusion; and 2 within 3 days of reperfusion) and another 2 animals failed to develop MI. Thus, 19 animals were available for serial studies and were followed for 72 hours. On CMR performed at 3 days post reperfusion, persistent microvascular obstruction (MVO) was evident in 17 animals. IMH was evident in 14 (74%) animals (IMH+group); the other 5 animals were identified as IMH−.

### Post-Reperfusion Troponin Kinetics in Canine Subjects Closely Resemble Findings in Reperfused Acute MI

Aggregate [hs-cTn-I]levels across all animals at baseline; immediate after reperfusion; and at 1, 3, 7, 15, 24, 48, and 72 hours after reperfusion from IMH+ and IMH− animals are shown in FIG. 5A. Prior to reperfusion (during ischemia), both hemorrhagic and non-hemorrhagic animals did not show any difference in [hs-cTn-I](p=0.21). However animals positive for hemorrhagic MI is characterized by a very rapid rise in troponin levels ([hs-cTn-I]) in the early time points of post-reperfusion period (0-4 hours, p<0.05) and a rapid fall after 48 hours of reperfusion.

In contrast, in the non-hemorrhagic group there was no difference in [hs-cTn-I]over the first 4 hours (p=0.12); however, there was marked difference at 24 hours (p=0.003) post-reperfusion. Animals without hemorrhagic MI showed a gradual rise in [hs-cTn-I]during the initial 24 hours and gradual fall thereafter. The divergence of [hs-cTn-I] kinetics in initial 3 hours post-reperfusion is without ambiguity. Time for [hs-Trop-I] to peak in hemorrhagic animals was at 3.5±0.37 hours while [hs-cTn-I] of non-hemorrhagic animals peaked at 16.8±1.6 hours post reperfusion. Notably the peak [hs-cTn-I]was >3.5-fold higher (259.72±15.44 ng/ml (hemorrhagic) vs 72.95±10.66 ng/ml (non-hemorrhagic)) and >12 hours earlier in the hemorrhagic animals compared to the non-hemorrhagic counterparts.

Aggregate Rate [hs-cTn-I]levels across all animals at baseline, immediately after reperfusion, and at 1, 3, 7, 15, 24, 48, and 72 hours after reperfusion are analyzed. Similar observations were made with Rate [hs-cTn-I] in canine with hemorrhagic MI as in human patients with Rate [hs-cTn-I]peaking at approximately at the same time point following reperfusion. In animals without hemorrhagic MIs, a slow and low magnitude Rate [hs-cTn-I] with a weak peak was observed at 24 hours. Specifically peak Rate [hs-cTn-I]was: 66.28 ng/ml/hr (hemorrhagic canine) vs 8.86 ng/ml/hr (non-hemorrhagic canine). Peak Rate [hs-cTn-I]occurred at 1.71±0.25 hours in hemorrhagic animals compared to 6.6±2.1 hours in non-hemorrhagic animals. AUC analysis of Rate [hs-cTn-I]showed that the earliest time to differentiate between hemorrhagic and non-hemorrhagic animals was as early as 1-hour post-reperfusion with AUC: 0.99, sensitivity: 93%, specificity: 80%, positive predictive value: 92%, and negative predictive value: 83%.

The correlation between IMH volume as determined from T2*CMR and peak [hs-cTn-I] and Rate [hs-cTn-I] are shown. Regression analysis of [hs-cTn-I] with hemorrhage volume showed highest correlation at 3 hours (R²=0.92).

### Hemorrhagic MI Identified Based On Cardiac Troponin Kinetics Shows Microvascular Rupture and Extravasation of RBCS Into the Interstitium

On histopathological examination of the infarct area, significant disruption of basement membrane and extravasation of RBCs were evident in hemorrhagic MI but not in Non-hMI case. Red blood cells (RBCs) were dispersed in the interstitial space amongst infarcted cardiomyocytes in hMI but were absent in the infarct area of Non-hMI. These histological findings validate the presence of acute microvascular rupture associated with intramyocardial hemorrhage as the cornerstone tissue injury in hMI, supporting findings on T2*CMR.

### Discussion

Hemorrhagic transformation of MI has emerged as a key contributor to reperfusion injury that can potentially double MI size within the first 24 hours of reperfusion and accelerate adverse remodeling [8,11] precipitating chronic heart failure. However, diagnosing hemorrhagic MIs is challenging because the current standard relies on CMR, which is broadly limited by accessibility and the requisite delay post PCI (3-5 days) for optimal imaging conditions [12]. This limits the identification/management of STEMI patients post PCI and impedes the development of therapies to mitigate the acute and progressive cardiac damage observed with hemorrhagic MI. Accordingly, there is a critical diagnostic gap in the field to rapidly detect hemorrhagic MIs based on a noninvasive and universally accessible approach.

Herein we demonstrated that cardiac troponin I—the same blood protein that is used to detect acute MI—can be used to diagnose hemorrhagic MI following primary PCI. We performed hourly, time-resolved sampling of pre- and post-PCI blood from STEMI patients and assayed them for [hs-cTn-I] with determination of hemorrhage status based on CMR prior to discharge. To overcome potential confounders (comorbidities, ischemic duration and standard of care medications), we also performed large animal studies under highly controlled conditions with both CMR and histology serving as ground truth for hemorrhage. Our results showed that [hs-cTn-I] kinetics can be used to accurately identify and size intramyocardial hemorrhage within the first few hours after reperfusion. In cases when multiple blood samples cannot be obtained in the early hours post PCI, our data supports the notion that a single blood draw for [hs-cTn-I] within the 48 hours of PCI has excellent diagnostic potential for even a delayed determination of hemorrhagic MI. Our findings here open the door for improved clinical management of the acute MI patients, development of therapeutic strategies targeting hemorrhagic MI patients, classification of MI patients for differential assessment of ongoing clinical trials, or even to screen MI patients for closer examination with any one of the advanced imaging approaches, including CMR.

The limited access to discern intramyocardial hemorrhage following PCI and its recently identified relation to rapid loss of salvageable myocardium [6] has made it difficult in the literature to interpret the longstanding observation in patient-to-patient variability in post PCI troponin kinetics. As cardiomyocytes are damaged, the cytosolic pool of troponin is released into the bloodstream leading to high plasma concentration of cardiac troponin, which can be detected by biochemical assays. Following proteolytic cleavage into smaller fragmental subunits, troponin is cleared via glomerular filtration and salivary barrier exchange [13], contributing to the observed 2-hour half-life of troponin-I [14].

As the washout of the troponin is flow-dependent, one line of thought is that the appearance of troponin is disrupted acutely by impaired myocardial blood flow. This can certainly contribute to time-dependent increase in troponin-I following as ischemia is relieved by reperfusion. However, this cannot explain differential troponin kinetics observed between hemorrhagic and non-hemorrhagic conditions in the post PCI period because the rapid rise in troponin in hemorrhagic MIs is counter to notion of reduced perfusion. If this were the case, the troponin peak in hemorrhagic MIs should occur much later than in non-hemorrhagic cases, which is not the case. As recently demonstrated [6], sudden appearance of hemorrhage following reperfusion imparts another wave of abrupt loss of myocardium contributing to a rapid rise in [hs-cTn-I] in blood. Nonetheless, the exact mechanism contributing to the fast versus slow rise of [hs-cTn-I] in hemorrhagic versus non-hemorrhagic MIs remains to be investigated. Further, EARLY ACS and SYNERGY trials have reported that 41.9% post-PCI patients with index hospitalization for ACS had new elevation of cardiac troponin within 24 hours after PCI. This 24-hr elevation of cardiac troponin correlated with an increased mortality as compared to patients with stable or decreased 24-hr cardiac troponin [15, 16]. However, the factors contributing to the differences in 24-hr cardiac troponin was not understood. In addition, in a recent study on 578 STEMI patients undergoing PCI cardiac troponin values were positively correlated with long-term LV systolic dysfunction [17]. Additionally, in patients with acute myocardial infarction (AMI), peak cTnI levels have shown superiority over traditional biomarkers like creatine kinase-MB, emphasizing their significance in risk assessment and clinical management (18, 19). Moreover, the elevation of cTnI after successful percutaneous coronary angioplasty has been associated with potential long-term prognostic value (20). The European Society of Cardiology (ESC) guidelines highlight the pivotal role of troponin testing in managing patients with ST-elevation MI (21).

Beyond the initial diagnosis, even minor increases in cTn levels have been linked to poorer prognosis in patients with unstable angina and chest pain (22, 23). Fixed-time high-sensitive troponin measurements have emerged as predictors of infarct size, impaired left ventricular function, and adverse outcomes in patients undergoing percutaneous coronary intervention (24), as well as potential for reperfusion injury (25), underscoring the importance of troponin readouts in the post reperfusion period. Together with previous reports that CMR-based evidence of hemorrhage contributes to adverse outcomes, along with the relationship between hemorrhage and cardiac troponin as demonstrated here, post PCI troponin elevations driving adverse events is likely best explained by the hemorrhagic transformation of MI. Our findings here support the notion that future investigations exploring post PCI complications can be empowered by an opportunity to investigate the influence of extensive microvascular injury from hemorrhage based on post reperfusion troponin kinetics.

The impact of infarct size before PCI on hemorrhagic MI transformation and subsequent differences in troponin kinetics is a topic of significance and complexity. Several key pieces of evidence suggest that infarct size before PCI may not be the primary driver of the divergence in troponin kinetics in hemorrhagic MI patients. Studies such as the EARLY ACS and SYNERGY trials have reported a high prevalence of new cardiac troponin elevation within 24 hours after PCI, correlating with increased mortality. This elevation is independent of pre-reperfusion troponin levels, indicating that infarct size may not be the sole determinant. Furthermore, research has highlighted the role of IMH in infarct expansion and reperfusion injury, specifically in hemorrhagic MI. This phenomenon contributes significantly to troponin release even when controlling for the area at risk. As half-life of troponin is 2 hours, the dynamic relative change in troponin has to be correlated with a real-time window of dynamic cardiomyocyte injury and not with remote time-scaled etiology. Therefore, attributing troponin kinetics solely to the initial infarct size oversimplifies the complex interplay of pre-reperfusion infarction, post-reperfusion IMH-mediated cardiomyocyte injury and post-IMH infarct expansion. The diverse troponin levels among AMI patients, including those with and without hemorrhagic transformation, likely result from unique pathophysiological processes specific to each subset. The presence of histopathological evidence in our preclinical model, such as the presence of red blood cells and disrupted microarterioles in the infarct area of hemorrhagic MI, further supports the notion that infarct size alone cannot explain the troponin kinetics divergence. In our meticulous hourly measurement of troponin alongwith tissue characterization by cardiac MR, removes confounding perception of wide inter-patient variability of post-reperfusion troponin levels and explains differential tissue level changes. Infact, this clarifies the earliest timepoint of inter-patient variations in pathophysiological course of infarcted myocardium and subsequent troponin release kinetics.

In addition to investigations in STEMI patients, we also explored the troponin-I kinetics in controlled, clinically relevant, animal models. This provided an opportunity to systematically validate the observations in the backdrop of heterogenous clinical comorbidities that are not present in our animal models. However, despite our findings between patients and canine models being remarkably similar, there were some differences. Whether these observations are due to differences in species, existence of comorbidities in patients, medications administered, or CMR conditions remains to be investigated.

Finally, our acute outcomes trial revealed a significant association between hMI and in-hospital mortality, with a substantial increase in odds, thus highlighting the critical importance of identifying and addressing hMI early in the clinical setting. Furthermore, our analysis demonstrated a heightened vulnerability of hMI patients to adverse events within the critical 30-day post-hospitalization period, with hMI significantly increasing the risk of 30-day mortality. The protective effects of specific medications, such as aspirin and P2Y12 inhibitors during percutaneous coronary intervention (PCI) procedures, were evident in both in-hospital and 30-day outcomes. This information can guide healthcare providers in optimizing treatment strategies and reducing adverse events in hMI patients. Additionally, the study explored 30-day rehospitalization, acute heart failure, arrhythmia events, and outcomes related to stable and unstable angina. The multifactorial nature of these outcomes underscores the complexity of patient management and the need for tailored care strategies.

These comprehensive findings contribute to the growing body of knowledge on hMI and provide essential guidance for risk assessment, treatment, and patient care, ultimately enhancing the quality of critical care management of acute MIs.

Our data is highly supportive of the use of post PCI [hs-cTn-I] kinetics to diagnose hemorrhagic MI. We did not evaluate troponin kinetics with the same temporal resolution in animals as we did in human patients, for animal welfare reasons. In spite this difference, [hs-cTn-I] kinetics between patients and canine were remarkably similar.

Overall, rapid elevation in cardiac troponin in the first few hours of PCI reflects hemorrhagic transformation of STEMI. Temporal sampling of blood for the assessment of post PCI troponin-I kinetics can be used to accurately detect and size intramyocardial hemorrhage. This introduces a clinical role for post PCI troponin kinetics as a noninvasive blood-based marker of microvascular damage which can aid in acute management, risk stratification and development of therapeutics for the most vulnerable MI patients.

Pre-revascularization troponin kinetics is an important blood marker of ischemic injury but post-revascularization, it can reflect hemorrhagic transformation of myocardial infarction, thus expanding its role in diagnosis and patient monitoring following reperfusion therapy. We conceive uses of post-reperfusion troponin kinetics for diagnosing hemorrhage, management of vulnerable STEMI patients, and towards the development of therapeutics to mitigate the damage associated with hemorrhagic myocardial infarction.

Overall, we studied ST-elevation MI patients (n=120) and validated the findings in canines (n=25). Blood samples were drawn pre- and post-reperfusion every hour up to 12 hours (then coarsely to 48-hours) in patients; and 1,3,7,15,24,48,72 hours post-reperfusion in animals. We examined [hs-cTn-I] kinetics in hemorrhagic and non-hemorrhagic subjects, its relation to hemorrhage volume (based on CMR), and determined the parameters for diagnosing hemorrhagic MI. Following reperfusion, [hs-cTn-I] between hemorrhagic and non-hemorrhagic MIs were divergent: peak [hs-cTn-I]was >10-fold in patients. Earliest time point to diagnose hemorrhagic MI was 1-hour post-reperfusion ([hs-cTn-I]cut-off: 92.68 ng/ml; sensitivity: 94%; specificity: 89%; area-under-the-curve: 0.95). At later time points, [hs-cTn-I]cut-offs varied with only mild decrease in diagnostic performance. By 48-hours, optimal cut-off was 19.23 ng/ml (sensitivity: 80%; specificity: 80%; area-under-the-curve: 0.85). Peak [hs-cTn-I]strongly correlated with hemorrhage volume (R2>0.80). Findings were similar in canines. Post-reperfusion [hs-cTn-I]can accurately diagnose hemorrhagic MI. This defines post-reperfusion [hs-cTn-I] as a new point-of-care blood marker in acute care management, risk stratification and development of therapeutics.

### Serum Troponin and CMR Analysis

T2*CMR, being the gold-standard method, was employed as the ground truth to retrospectively categorize reperfused MIs into hemorrhagic and non-hemorrhagic groups, and to quantify the volume of IMH (% LV). MIs with IMH volume<4%, as determined by T2*CMR, were classified as non-hemorrhagic [6]. The temporal kinetics of [hs-cTn-I] in hemorrhagic and non-hemorrhagic MI subjects were assessed using the time series of [hs-cTn-I]. Concurrently, the rate of change in [hs-cTnI]relative to time (t) after reperfusion in hemorrhagic and non-hemorrhagic MI was computed as

Rate [hs-cTn-I]=([hs-cTn-I(t)]-[hs-cTn-I(−1)])/t, where [hs-cTn-I(t)] is [hs-cTn-I] at time t after reperfusion, [hs-cTn-I](−1) or [hs-cTn-I]o is the [hs-cTn-I]pre-PCI. Note that in this definition, hs-cTn-I(0)] is [hs-cTn-I]immediately after reperfusion. Linear regression analysis was carried out to ascertain the relationships between INM (% LV) and peak [hs-cTnI], as well as Rate [hs-cTn-I]. Receiver operating characteristic (ROC) analysis was conducted to establish the cut-off values and diagnostic accuracy of hs-cTn-I and Rate [hs-cTn-I] for detecting hemorrhagic MI at various time points after reperfusion.

Clinical Trial: 30-day Clinical Outcome

In our retrospective multicenter registry analysis (MIRON-ACUTE, ClinicalTrials.gov Identifier: NCT0000000), inclusion criteria aimed to investigate STEMI with primary PCI and post-PCI troponin levels. We meticulously matched time-scaled post-PCI troponin measurements with our diagnostic table (Table 2). Subsequently, we categorized these patients into two distinct groups: hemorrhagic and non-hemorrhagic MI. The primary outcome measures included in-hospital mortality, 30-day mortality, and 30-day re-hospitalizations. Within the re-hospitalization category, we examined the causes for hospitalization, comprising unstable angina, stable angina, reinfarction, acute heart failure, arrhythmia, and stroke.

### Statistical Analysis

In this study, we conducted comprehensive statistical analyses to assess the predictive power and associations of our data using the SAS software (version 9.4, SAS Institute Inc., Cary, NC). To evaluate the discriminatory ability of our diagnostic model, we performed Receiver Operating Characteristic (ROC) analysis, including the calculation of the area under the curve (AUC). Additionally, we conducted Odds Ratio (OR) analysis to investigate the relationships between co-variables and hMI outcomes. Logistic regression models were fitted to estimate ORs and their corresponding 95% confidence intervals. The Odds Ratio analysis was matched for critical covariates, including demographics, co-morbidities, pre-admission medications, procedural medications, and discharge medications. This comprehensive matching process ensured that the ORs accurately reflected the associations between the variables of interest and the outcome, while minimizing the potential influence of confounding factors. All statistical tests were two-sided, and a p-value less than 0.05 was considered statistically significant.

Further description of the examples include:

In a prospective cohort of STEMI patients (n=201) undergoing PCI, a troponin-based test was developed and validated against gold-standard T2*CMR to derive a blood biomarker for hemorrhagic STEMI. Based on this test, a multisite cohort of reperfused STEMI patients (n=6180), dichotomized for IMH status, was used to predict primary acute outcomes (mortality, readmission, acute HF). This data was augmented with Cerner National records (spanning 114 hospitals across the US) to assess the regional prevalence of hemorrhagic STEMIs in the US.

RESULTS: Sensitivity, specificity and AUC for the troponin-based biomarker for hemorrhagic STEMI were all >0.8. Peak troponin concentration was correlated with IMH volume (R²=0.89, p<0.001). Acute outcomes in hemorrhagic STEMI (n=1323) showed increased odds of in-hospital mortality (Adj-OR: 2.8; 95% CI: 2.1-3.7, AUC=0.75), 30-day mortality (Adj-OR: 2.9; 95% CI: 2.2-3.7, AUC=0.74), 30-day readmission (Adj-OR: 1.5; 95% CI: 1.2-1.9, AUC=0.65), and 30-day acute HF (Adj-OR: 2.6; 95% CI: 1.7-4.1, AUC=0.75); p<0.0001, for all. Combined records of STEMI (n=24,181) demonstrated that the greatest prevalence of hemorrhagic STEMI was in the southeastern region of the United States. Hemorrhagic STEMI can be diagnosed based on a blood biomarker. They carry a significantly elevated risk of adverse acute outcomes and are most populated in the “Heart Failure Belt” of the United States. This biomarker may be used as a benchmark for therapeutic, prophylactic agents in mitigating IMH to improve post-STEMI survival.

### Specifically:

We developed post-PCI troponin as a diagnostic biomarker for hemorrhagic MI in a prospective trial, MIRON-TROP (NCT05872308), along with pre-clinical validation studies and then used it to study acute clinical outcomes in hemorrhagic and non-hemorrhagic ST-elevation MI patients. Specifically, we investigated the outcomes in ST-elevation MI patients (hemorrhage status identified based on time-dependent post-PCI troponin cutoffs from MIRON-TROP) using data from 7 hospitals, in a study referred to as MIRON-ACUTE (NCT06110351). To gain a broader perspective of the acute outcomes across the United States, we augmented MIRON-ACUTE data with Cerner National (spanning 114 hospitals across the US, albeit with limited mortality data). This allowed us to examine the prevalence of hemorrhagic MI and acute clinical outcomes (without mortality) across the United States in a study referred hereinafter as MIRON-National.

### Development of Serum Troponin as a Biomarker for Hemorrhagic MI Diagnostics

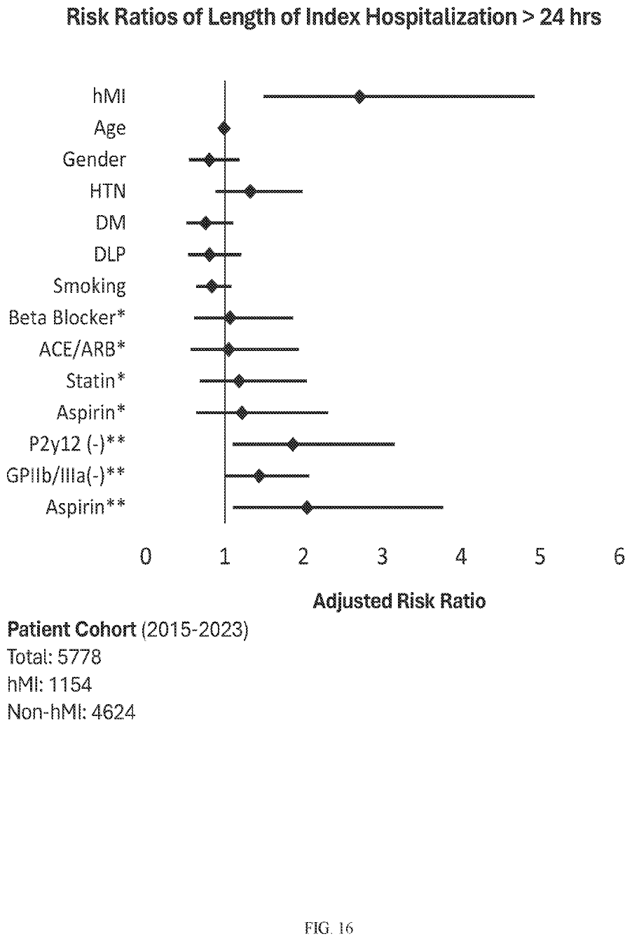

Clinical Trial (MIRON-TROP): We performed a prospective multicenter observational study and enrolled consecutive STEMI patients undergoing primary PCI between June 2022 and November 2023 (MIRON-TROP, NCT05872308). Patients with hemodynamic instability or contraindications for CMR were excluded. Patient selection details are captured in flow chart (FIG. 16A). Blood samples were obtained from patients (n=201) upon arrival at ER (0 hrs), immediate post-reperfusion (−1 hr) and every hour up to 12 hours, and then 16-, 20-, 24- and 48-hours post-PCI. Concentration of high-sensitivity cardiac troponin-I ([hs-cTn-I]) were assayed (ACEESS immunoassay systems, Beckman Coulter, California, USA). CMR was performed using MAGNETOM Sola (Siemens Healthineers, Germany) at 48-72 hours post-PCI.

Pre-Clinical Studies: A canine study (n=25) was performed as approved by Institutional Animal and Usage Committee to assess any potential differences related to patient comorbidities and medications. Blood samples were collected at multiple time points (during ischemia (noted as −1 hr), immediately after reperfusion (0 hr), and at 1,3,7,15,24,48,72 hrs post reperfusion) and [hs-cTn-I] were assayed as in MIRON-TROP.

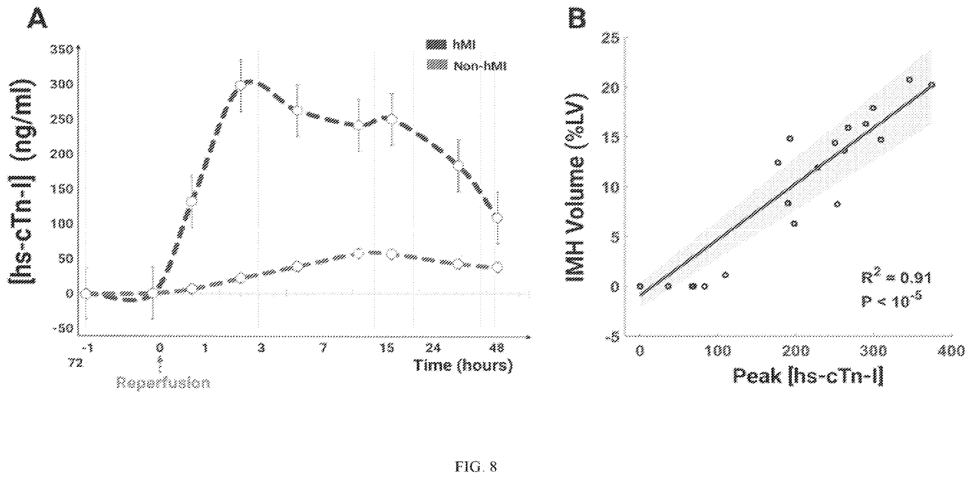

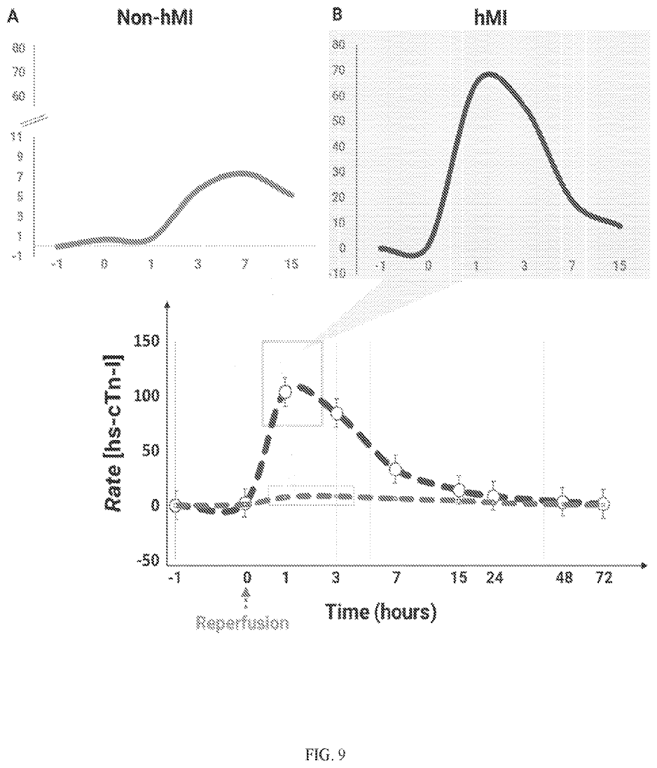

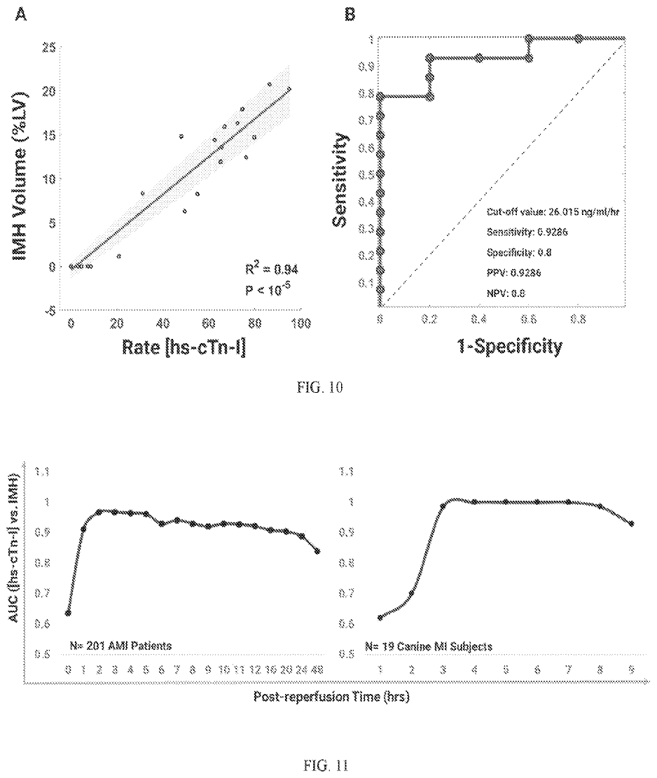

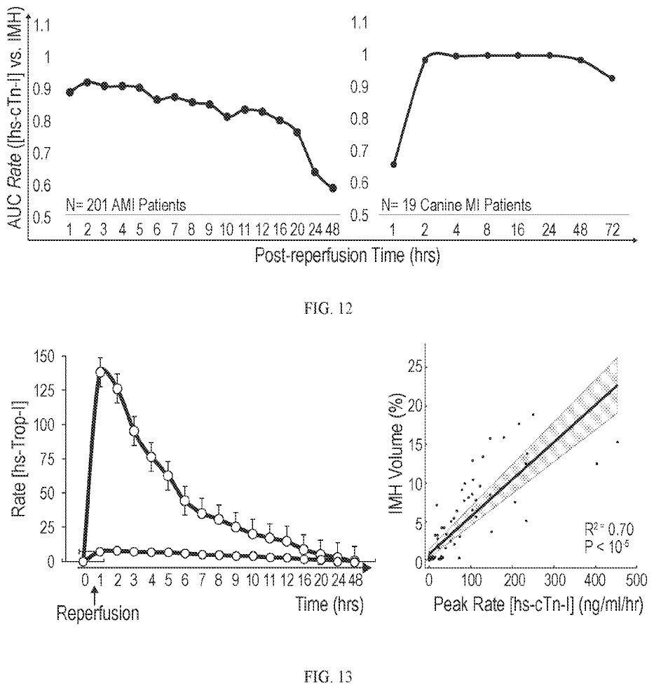

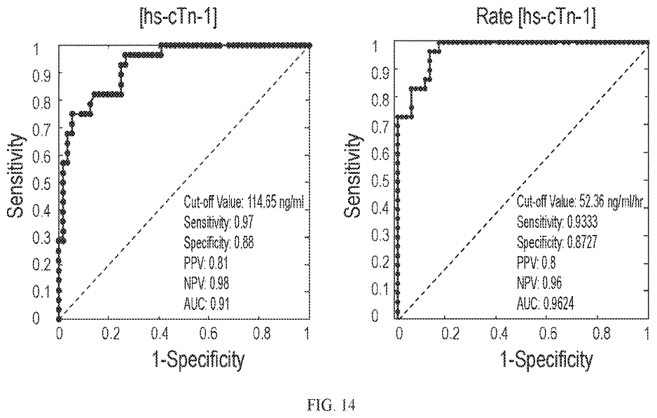

Validation: Troponin-based detection of hemorrhagic MI was validated in two ways, one using CMR as the ground truth and the other using animal histology. T2*CMR served as the ground truth to retrospectively categorize reperfused MIs into hemorrhagic and non-hemorrhagic groups, and to quantify the volume of III (% LV). MIs with IMI volume<4%, as determined by T2*CMR, were classified as non-hemorrhagic [6]. The temporal kinetics of [hs-cTn-I] in hemorrhagic and non-hemorrhagic MI subjects were assessed using the time series of [hs-cTn-I]. Rate of change in [hs-cTnI]was also assessed. Receiver operating characteristic (ROC) analysis was conducted to establish the cut-off values and diagnostic accuracy of hs-cTn-I for detecting hemorrhagic MI at various time points after reperfusion. CMR exam included assessment of cardiac function, determination of myocardial infarction and hemorrhage (FIG. 8).

Acute Clinical Outcomes in Hemorrhagic MI Patients Based on Post-PCI Troponin Diagnostics We performed retrospective multicenter registry analysis (MIRON-ACUTE; NCT06110351) to assess 30-day clinical outcomes in hemorrhagic and non-hemorrhagic STEMI patients. We meticulously matched time-scaled post-PCI troponin measurements from the electronic medical records with our diagnostic table as determined by the post-PCI hourly troponin-I cut-offs from MIRON-TROP. We data from MIRON-ACUTE (FIG. 16B) for patient selection) to closely study acute outcomes, as it captured angiographic and cardiac catheterization parameters and history of pre-PCI, as well as in-hospital and discharge medications, and mortality details. In MIRON-ACUTE, primary outcomes were acute mortality (in-hospital and 30 day) as well as 30-day hospitalizations. Within the re-hospitalization category, we examined the causes for hospitalization, comprising unstable angina, stable angina, reinfarction, acute heart failure, arrhythmia, and stroke. The composite data across the US (MIRON-National) is summarized in FIG. 16C (patient selection) and Table 1C (patient characteristics)). Given that the mortality data in the Cerner National database was sparse, the primary outcome measures evaluated in MIRON-National were limited to 30-day re-hospitalizations and their causes.

### Statistical Analysis

Statistical analysis for MIRON-TROP was performed using IBM SPSS Statistics 23 (IBM Corp., Armonk, New York). Normality of continuous data was determined by using the Shapiro-Wilk test and quantile-quantile plots. Continuous variables were expressed as mean±standard error. Categorical variables are reported as numbers and percentages. Differences between hemorrhagic and non-hemorrhagic MI groups were compared using Student's t-test. Chi-square test was used to compare differences in categorical variables. [hs-cTn-I]across different time points were compared using 2-way repeated measures ANOVA. Continuous variables were correlated using Pearson's correlation coefficient. ROC analysis was used to compute the area-under-the-curve (AUC) taking CMR-diagnosis as the ground truth. We investigated the predictive power and associations of our data for MIRON-ACUTE and MIRON-National with SAS (version 9.4, SAS Institute Inc., Cary, NC). To evaluate the discriminatory ability of our diagnostic model, we performed Receiver Operating Characteristic (ROC) analysis, including the calculation of the area under the curve (AUC). We performed Odds Ratio (OR) analysis to investigate the relationships between co-variables and outcomes related to hemorrhagic MI. Logistic regression models were fitted to estimate ORs and their corresponding 95% confidence intervals. The OR analysis was adjusted for critical covariates, including demographics, co-morbidities, pre-admission medications, procedural medications, and discharge medications and are reported as adjusted OR (Adj. OR). This ensured that the ORs accurately reflected the associations between the variables of interest and the outcome, while minimizing the potential influence of confounding factors. All statistical tests were two-sided, and a p-value less than 0.05 was considered statistically significant.

### Results

### Highly-Time Resolved Serum Troponin Kinetics in Revascularized STEMI Patients—MIRON-TROP

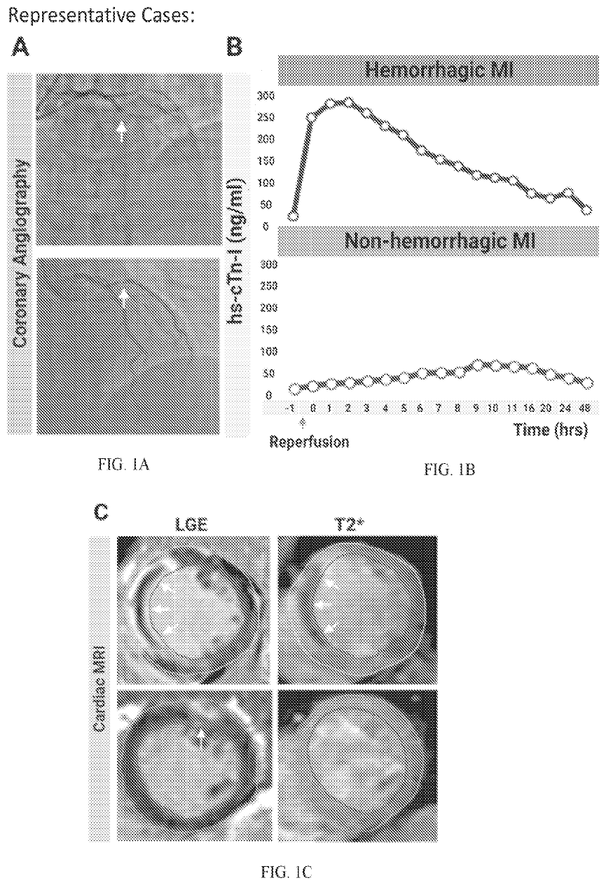

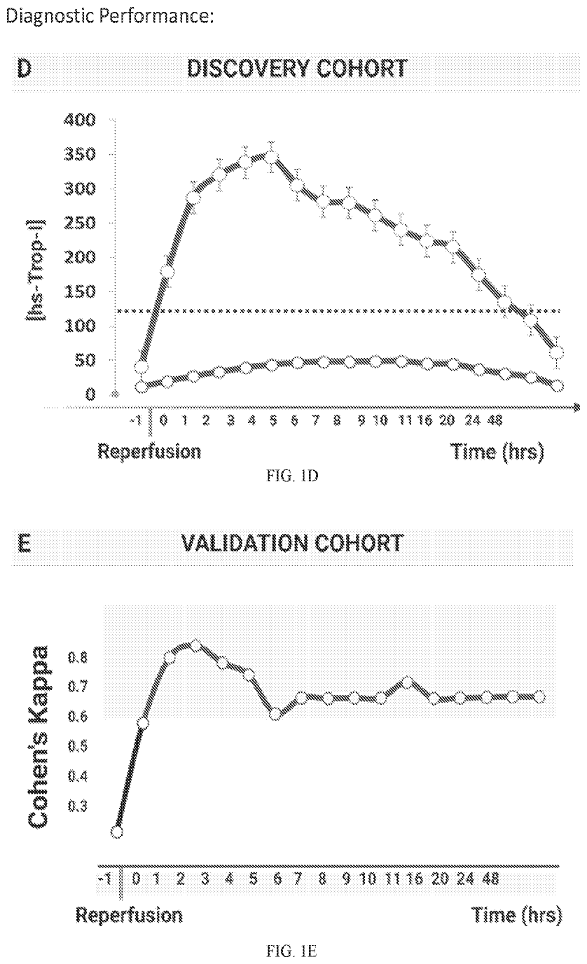

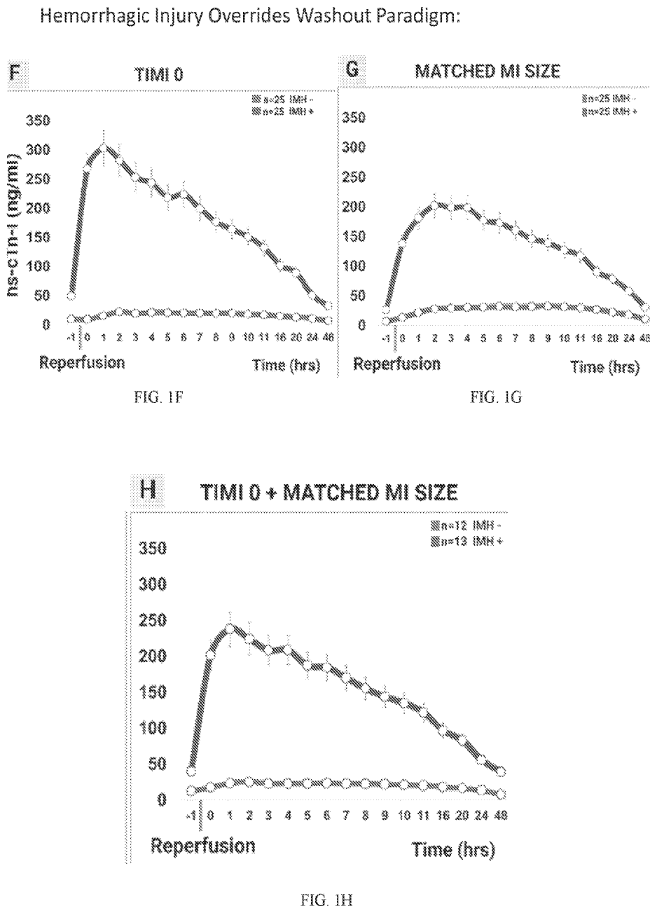

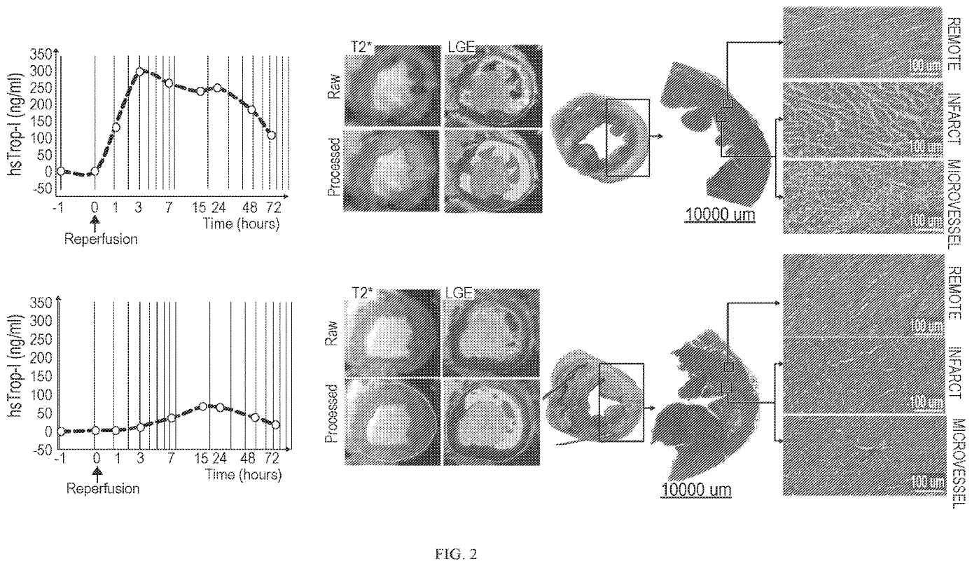

A total of 221 STEMI patients revascularized with primary PCI were prospectively enrolled in the study. Due to clinical constraints, consent withdrawal, logistical constraints and adverse clinical course resulting in challenges associated with accurate and timely blood sampling, data from 9.1% (n=20) were not included. Based on CMR, participants were retrospectively classified into two categories, hemorrhagic MI (n=81) and non-hemorrhagic MI (n=120). Key findings were: Troponin Kinetics: Hemorrhagic transformation of acute MI leads to a rapid surge of cardiac troponin in blood within hours of reperfusion and peak [hs-cTn-I]correlates strongly with hemorrhage volume measured with CMR. Representative revascularized hemorrhagic and non-hemorrhagic STEMI patients identified with CMR, along with pre-PCI angiograms delineating the culprit coronary branches and corresponding highly time-resolved [hs-cTn-I] kinetics are shown in FIG. 1A-C. The hemorrhagic patient showed a sharp increase in [hs-cTn-I] within the 1-2 hours of reperfusion, compared to the non-hemorrhagic patient in whom the [hs-cTn-I]peak was gradual and peaked −12 hours after. Aggregate [hs-cTn-I]across all patients prior to PCI, immediately after reperfusion, and the hours after (up to two days) are shown in FIG. 1D. Prior to PCI, [hs-cTn-I] in hemorrhagic and non-hemorrhagic patients were not different (p>0.2). In the hemorrhagic MI group, [hs-cTn-I]peaked 6.5 hours earlier and 11-fold greater (4.02±0.78 hours with a peak of 348.16±37.07 ng/ml) than non-hemorrhagic MI (10.52±0.67 hours with a peak of 30.95±3.57 ng/ml). Regression between IMH volume (from T2*CMR) and peak [hs-cTn-I]averaged in discretization of 100 ng/ml is shown in FIG. 1E. Peak [hs-cTn-I]occurred 2-4 hours after PCI and was highly correlated with hemorrhage volume (R2=0.89, p<0.001).

Diagnostic Performance: Cardiac troponin-based detection of hemorrhagic MI is robust over the first 20 hours post reperfusion and diminishes mildly thereafter up to 48 hours. The sensitivity and specificity of [hs-cTn-I] for detecting hemorrhagic MI are greater than 80% over the first 20 hours post PCI. AUC associated with [hs-cTn-I]based detection of hemorrhagic MI was high (>0.9) for nearly up to 24 hours post PCI and decreasing only slightly thereafter by 48 hours PCI (FIG. 1E). Details on hemorrhagic MI diagnosis, particularly the time dependent [hs-cTn-I]threshold for diagnosing hemorrhagic STEMI is presented in the Table 2.

### Invasive Preclinical Validation of Troponin Kinetics for Detection of Hemorrhagic MI:

Hemorrhagic MI identified based on cardiac troponin kinetics in animals was similar to patients and showed microvascular rupture and extravasation of red blood cells into the interstitium. On histopathological examination of the MI area, significant disruption of basement membrane and extravasation of red blood cells were evident in hemorrhagic MI but in non-hemorrhagic MI. Representative cases with serum troponin-I kinetics, CMR and histology are shown in FIG. 2. Red blood cells were dispersed in the interstitial space amongst infarcted myocardium in hemorrhagic MI but were absent in the infarct area of non-hemorrhagic MI, establishing the relationship between serum troponin levels, CMR and histological evidence of hemorrhage.

### Acute Clinical Outcomes in Hemorrhagic MI-MIRON-ACUTE

MIRON-ACUTE comprised of a final cohort of 6,180 patients, with 1,323 patients deemed to be hemorrhagic and remining 4,857 patients non-hemorrhagic based on post-PCI troponin diagnostics. Key findings were: In-hospital Mortality: Hemorrhagic MI was strongly associated with an increased odds of mortality during index hospitalization, with Adj. OR of 2.795 (95% CI: 2.084-3.748, p<0.0001, AUC=0.7513). Age, when analyzed in terms of days, demonstrated a significant impact on in-hospital mortality, with each additional day of age contributing to 1.024 times higher odds of mortality (95% CI: 1.012-1.036, p=0.012). Conversely, gender showed no significant effect on mortality (Adj. OR: 1.109, 95% CI: 0.813-1.512, p=0.296). Patients with hypertension had 1.521 times higher odds of mortality (95% CI: 1.03-2.246, p=0.491), and those with diabetes had 1.714 times higher odds (95% CI: 1.257-2.336, p=0.457). Dyslipidemia had a protective effect on mortality, with an Adj. OR of 0.669 (95% CI: 0.482-0.928, p=0.187). Smoking (OR: 0.883, 95% CI: 0.683-1.143, p=0.2) and the use of beta-blockers (BB, Adj. OR: 1.687, 95% CI: 1.025-2.777, p=0.662) showed no significant associations with in-hospital mortality. Notably, the use of aspirin and P2Y12 inhibitors during PCI procedures had a protective effect on mortality (Aspirin Adj. OR: 0.343, 95% CI: 0.21-0.562, p<0.0001; P2Y12 Adj. OR: 0.35, 95% CI: 0.245-0.499, p<0.0001). In contrast, ACE inhibitors/ARBs, statins, GP IIb/IIIa inhibitors, and aspirin given during PCI procedures did not significantly impact mortality. 30-day Mortality: Hemorrhagic MI emerged as a significant risk factor, with an Adj. OR of 2.85 (95% CI: 2.199-3.693, p<0.0001, AUC=0.743), during the 30-day post-PCI period. Age displayed a significant impact, with each additional day associated with a 0.068% increase in the odds of 30-day mortality (p=0.022). Conversely, gender, hypertension, diabetes, dyslipidemia, smoking status, and beta-blockers showed no significant associations with 30-day mortality, suggesting their limited role as independent predictors. The use of aspirin and P2Y12 inhibitors during PCI procedures demonstrated a protective effect with Aspirin Adj. OR of 0.375 (95% CI: 0.258-0.543, p<0.0001); and P2Y12 of 0.390 (95% CI: 0.282-0.540, p<0.0001). Conversely, post-PCI medications, such as beta-blockers, ACE inhibitors/ARBs, statins, aspirin, P2Y12 inhibitors, and anticoagulants, did not significantly impact 30-day mortality.

30-day Rehospitalization: Hemorrhagic MI was associated with an increased odd of readmission, with a statistically significant Adj. OR of 1.523 (95% CI: 1.226-1.89, p=0.0001, AUC=0.6545). Age had a marginal impact on readmissions, with 1.008 times higher odds for each additional day of age (95% CI: 1.000-1.016, p=0.008). Gender demonstrated no significant effect (Adj. OR: 1.152, 95% CI: 0.934-1.422, p=0.218). Patients with hypertension and diabetes had Adj. ORs of 1.233 (95% CI: 0.95-1.6, p=0.283) and 1.023 (95% CI: 0.824-1.271, p=0.199) for readmission, respectively. Dyslipidemia (DLP), smoking, and the use of medications, including beta-blockers, ACE inhibitors/ARBs, statins, aspirin, and P2Y12 inhibitors, demonstrated varying influences on readmission rates.

30-day Acute Heart Failure (HF): Hemorrhagic MI showed a substantially higher risk for experiencing acute HF within 30 days, with an Adj. OR of 2.634 (95% CI: 1.683-4.121, p<0.0001, AUC=0.7466). Age had a notable impact, with 1.033 times higher odds of acute HF for each additional day of age (95% CI: 1.012-1.054, p=0.0021). Conversely, gender did not exhibit a significant effect (Adj. OR: 0.7, 95% CI: 0.407-1.202, p=0.1961). Patients with hypertension showed an Adj. OR of 1.091 (95% CI: 0.95-2.183, p=0.546), while those with diabetes had an Adj. OR of 0.98 (95% CI: 0.824-1.271, p=0.411) for readmission due to acute HF. The influence of other factors, including dyslipidemia, smoking, and medication usage (beta-blockers, ACE inhibitors/ARBs, statins, aspirin, and P2Y12 inhibitors), on acute HF rates was variable.

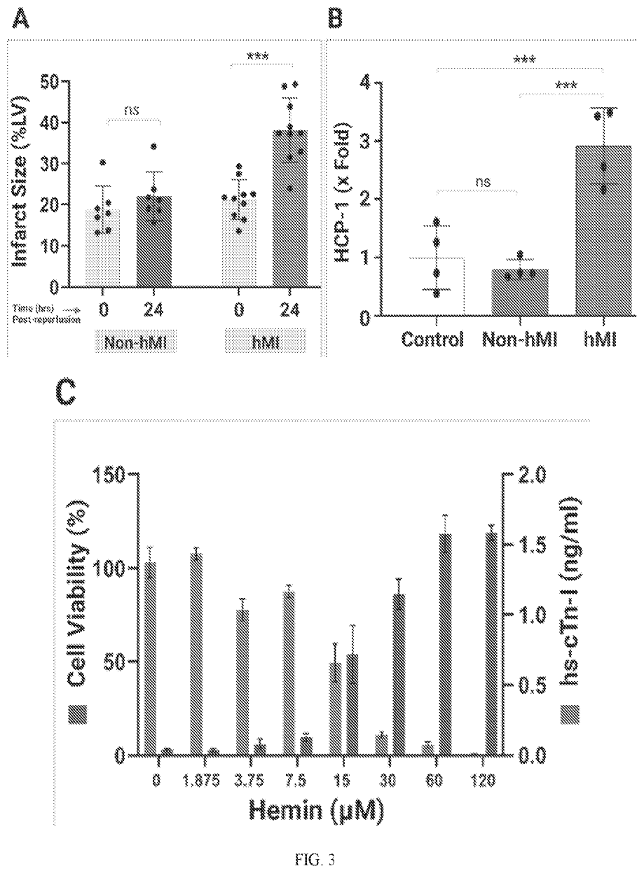

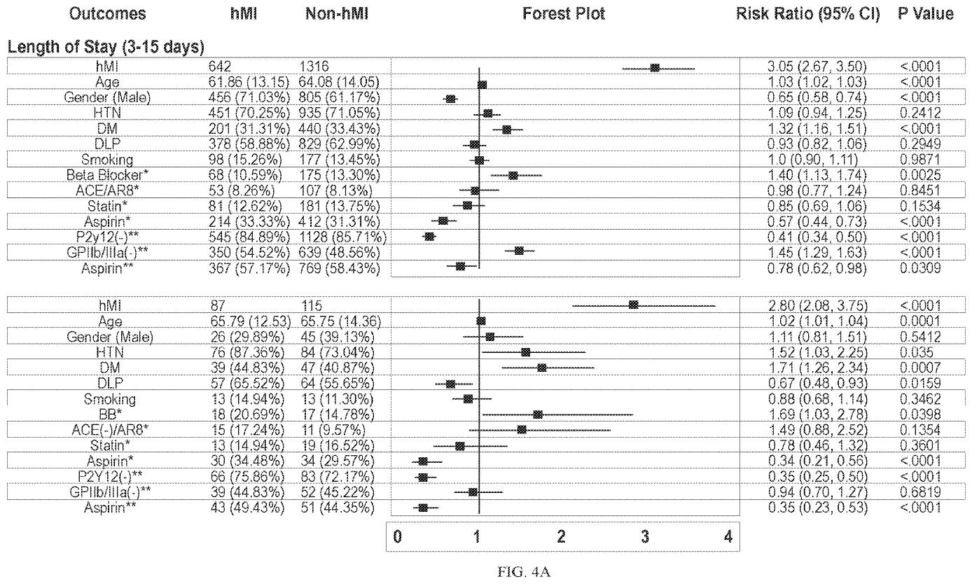

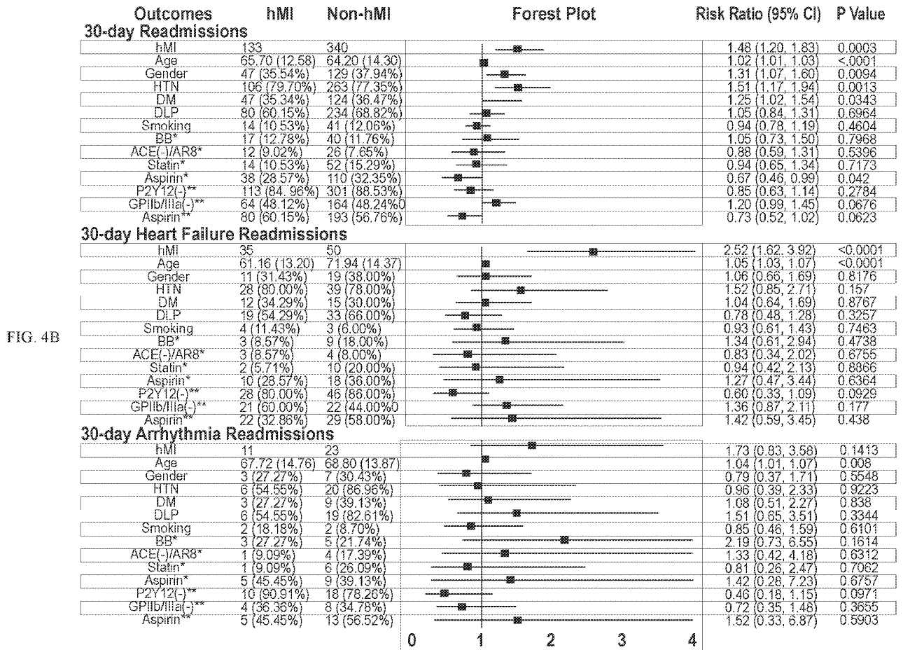

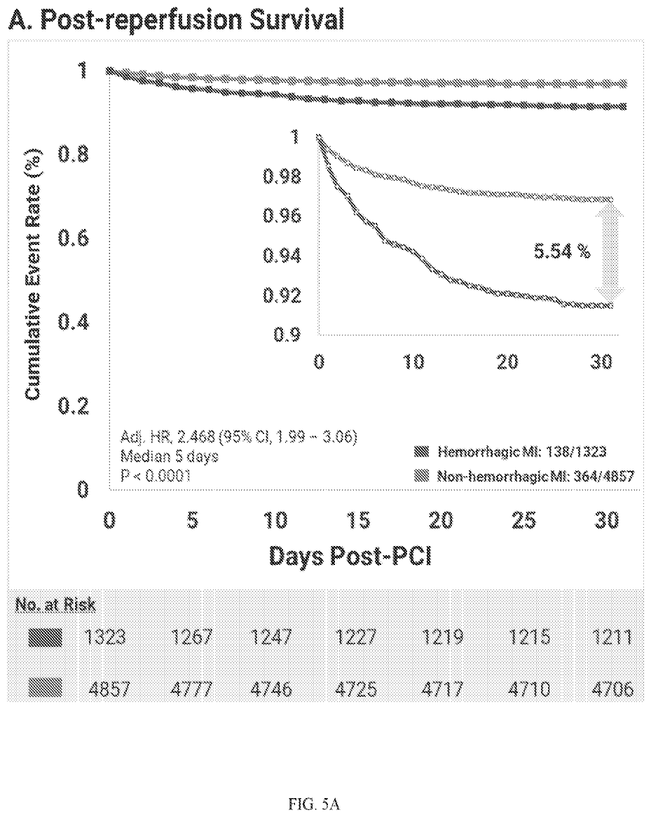

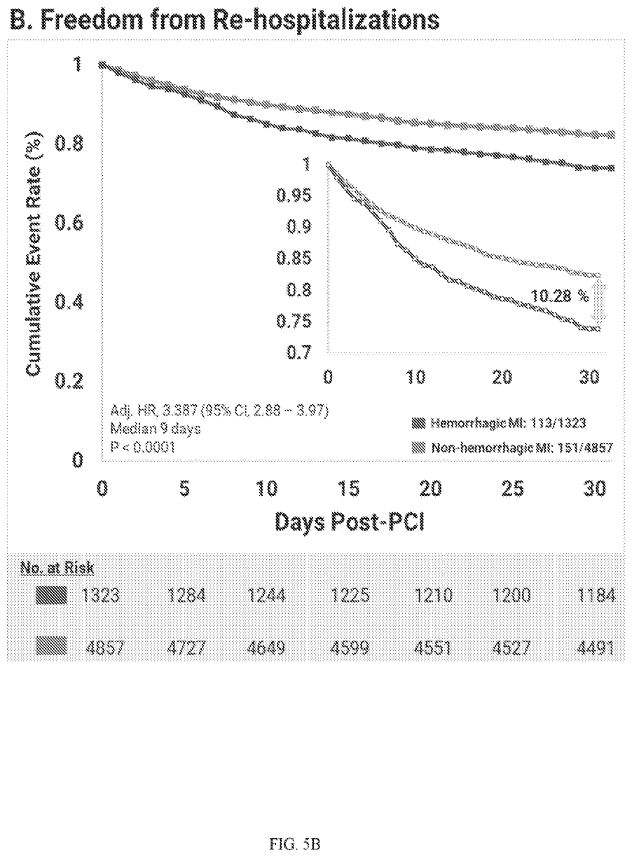

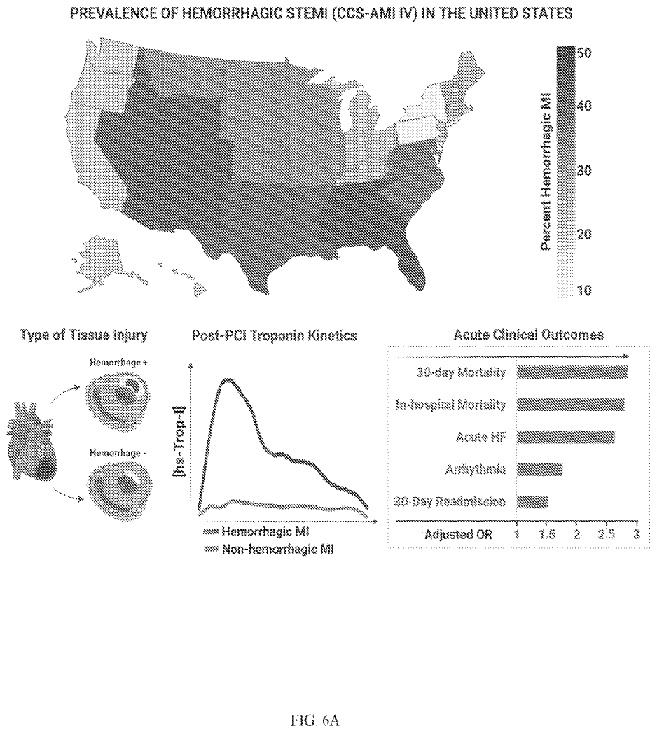

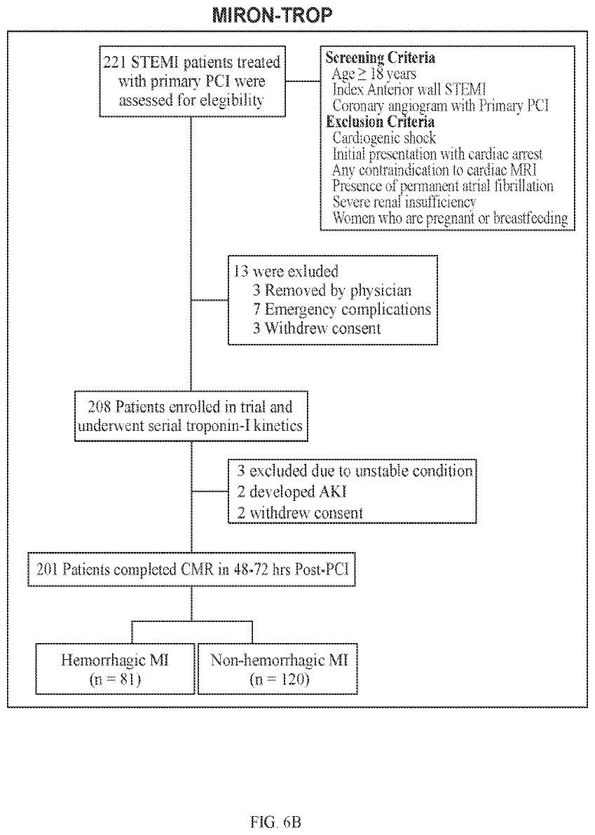

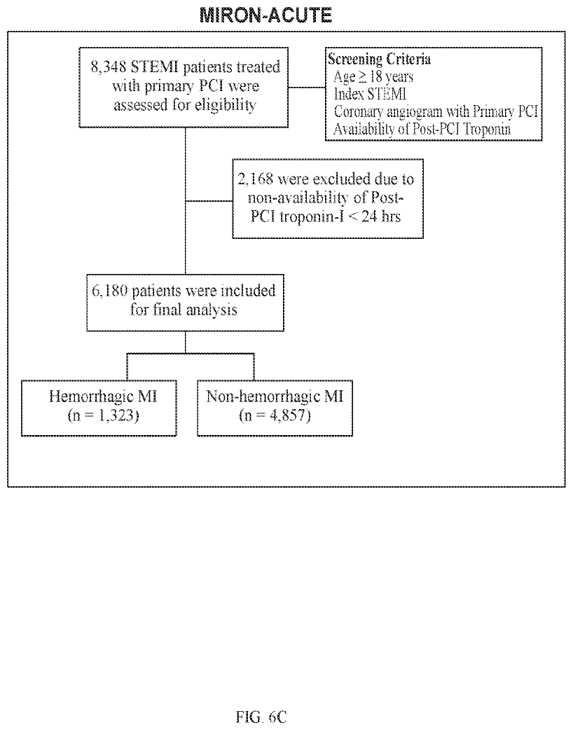

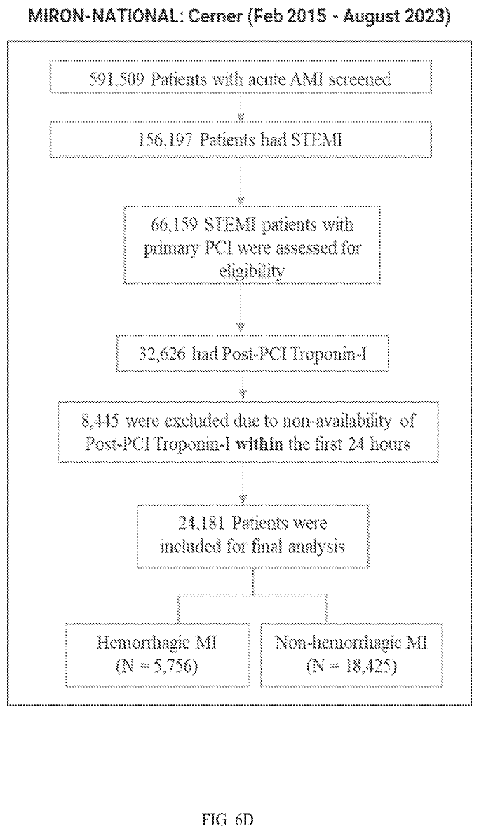

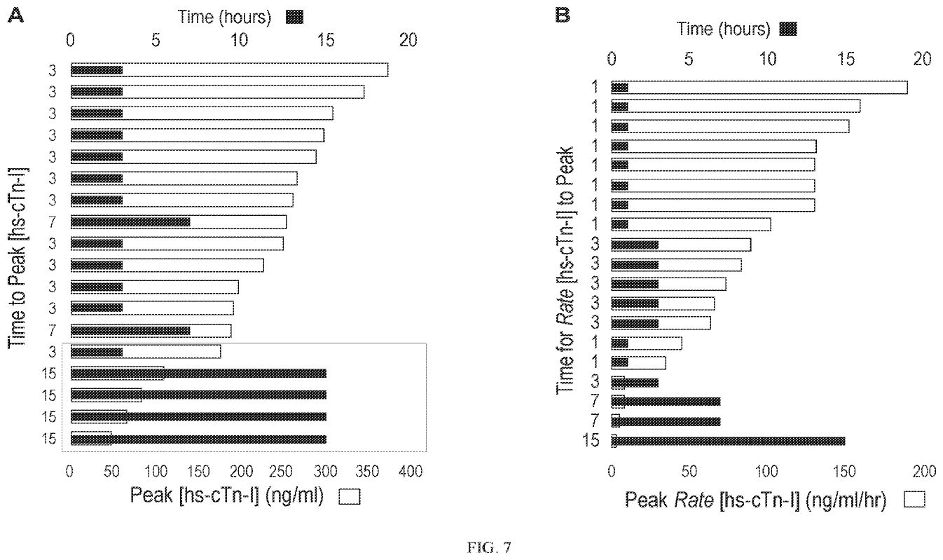

30-day Arrhythmia Events: Hemorrhagic MI exhibited a non-significant trend, with an increased Adj. OR of 1.757 (95% CI: 0.844-3.656, p=0.1319, AUC=0.7146), suggesting a high potential for association that did not reach statistical significance. Age remained a marginal contributor, with each additional day increasing the odds by 2.8% (Adj. OR: 1.028, 95% CI: 0.996-1.060, p=0.032), indicating a minor impact of age on arrhythmia-related readmissions. Other factors, gender, hypertension and diabetes were not found to have significant effects. Multi-variate Adj. OR are summarized in FIG. 3 and Kaplan-Meir survival curves from in-hospital and 30-day mortality are shown in FIG. 4. Other 30-day outcomes (stable angina, unstable angina, re-infarction and cerebrovascular stroke) were not significantly different between the hemorrhagic and non-hemorrhagic groups.

### Acute Clinical Outcomes in Hemorrhagic MI Patients—MIRON-National

MIRON-National comprised of a final cohort of 24,181 patients, with 5,756 identified to be hemorrhagic and 18,425 non-hemorrhagic based on troponin diagnostics. This enabled the establishment of the prevalence of hemorrhagic MI across the US Patient characteristics are captured in Table 1C. We observed a readmission rate of 8.5% across 30-day re-hospitalization across all subjects with 9.9% in hemorrhagic MI versus 8.1% in non-hemorrhagic MI (p<0.001). The Adj. OR of hemorrhagic MI was 1.28 (95% CI 1.17-1.41, p<0.001, AUC=0.594), signifying a 28% higher likelihood of readmission compared to non-hMI patients. Arrhythmia occurred in 3% of the overall cohort, with a higher prevalence towards the hemorrhagic MI group (4%) compared to the non-hemorrhagic MI group (2%). Notably, hemorrhagic MI emerged as a significant predictor, exhibiting Adj. OR of 1.48 (95% CI 1.25-1.74, p<0.001, 0.664), indicating a 48% increased likelihood of arrhythmia in hemorrhagic versus non-hemorrhagic MI patients. Further, among the total cohort, 3% experienced acute heart failure (AHF), with 4% in hemorrhagic patients versus 2% in the non-hemorrhagic group. Notably, hemorrhagic MI patients demonstrated a substantial increase in the odds for AHF compared to non-hemorrhagic MI patients (Adj. OR=1.701, 95% CI [1.447, 1.995], p<0.001, AUC=0.69). However, the hemorrhagic MI did not demonstrate a statistically significant association with stroke incidence (Adj. Adj. OR=1.134, 95% CI [0.673, 1.838], p=0.622, AUC=0.627).

### Discussion

Based on the prospective trial, MIRON-TROP, we demonstrated that cardiac troponin I—the same blood protein that is routinely used to detect acute MI prior to intervention—can be used to diagnose hemorrhagic MI following primary PCI. We then employed the time-dependent post-PCI troponin derived within the first 24 hours from MIRON-TROP to identify hemorrhagic MI patients using data registries to determine acute clinical outcomes across the United States. Our findings demonstrate that hemorrhagic MI patients carry a significantly elevated risk of acute mortality, 30-day rehospitalization, and acute heart failure. Collective evidence from the study also provides the first evidence of the prevalence of hemorrhagic MI across the United States.

The limited access to discern hemorrhagic transformation of MI following PCI has made it difficult to interpret the longstanding observation in patient-to-patient variability in post PCI troponin kinetics. As cardiomyocytes are damaged, cytosolic pool of troponin is released into the bloodstream leading to high plasma concentration of cardiac troponin and is cleared via glomerular filtration and salivary barrier exchange [8], contributing to the observed 2-hour half-life of troponin-I [9]. As the washout of the troponin is flow-dependent, it may suggest that troponin is disrupted acutely by impaired myocardial blood flow and contribute to time-dependent increase in troponin-I as ischemia is relieved by reperfusion. However, this cannot explain differential troponin kinetics observed between hemorrhagic and non-hemorrhagic conditions in the post PCI period because the rapid rise in troponin in hemorrhagic MIs is counter to notion of reduced perfusion. If this were the case, the troponin peak in hemorrhagic MIs should occur much later than in non-hemorrhagic cases, which is not the case. Our finding of rapid rise of [hs-cTn-I] in hemorrhagic versus non-hemorrhagic MIs, although remains to be rigorously investigated, is likely related to the rapid loss of myocardium post PCI with the abrupt appearance of hemorrhage within the MI zone, as recently shown [6].

The findings here also provide further insight into EARLY ACS and SYNERGY trials, which have reported that 41.9% post-PCI patients with index hospitalization for ACS had new elevation of cardiac troponin within 24 hours after PCI. This 24-hr elevation of cardiac troponin correlated with an increased mortality as compared to patients with stable or decreased 24-hr cardiac troponin [10, 11]. Another study investigating post PCI troponin in 578 STEMI patients demonstrated positive correlation between troponin and long-term LV systolic dysfunction [12]. Peak [hs-cTn-I]levels have also shown superiority over traditional biomarkers such as creatine kinase-MB, emphasizing their significance in risk assessment and clinical management [13,14]. Others have also that [hs-cTn-I]after successful PCI is liked to long-term prognosis ([15]), which reflected in the European Society of Cardiology (ESC) guidelines emphasizing the pivotal role of troponin testing in managing patients with ST-elevation MI ([16]). Our findings here support the notion that future investigations exploring post PCI complications can be empowered by an opportunity to investigate the influence of hemorrhage on post reperfusion troponin kinetics.

The 2.8-fold odd ratio of in-hospital mortality in hemorrhagic MI patients that we observed here highlights the importance of identifying and addressing hemorrhagic MI early in the clinical setting. Similarly, a multi-fold greater risk for 30-day mortality in hemorrhagic MI patients re-emphasizes the need for improved care for hemorrhagic MI patients. The protective effects of specific medications, such as aspirin and P2Y12 inhibitors during PCI procedures, as evidenced by the in-hospital and 30-day outcomes suggests that optimized treatment strategies can reduce adverse events in hemorrhagic MI patients. Additional studies are warranted to improve patient management and the need for tailored care strategies particularly for hemorrhagic MI patients.

We made significant efforts to minimize study limitations; yet still some limitations remain. Our studies suggest but do not overtly explore mechanisms for the observed evidence on the relationship between [hs-cTn-I] and hemorrhage. Our retrospective outcome studies also impart certain limitations, particularly limiting the ability to control for potential biases introduced by the selection of patients or data collection methods as used in the past. Given that post-PCI troponin values were only available at single time points, we were not able to estimate the extent of hemorrhage but only that they were hemorrhagic. This limited our ability to further characterize the magnitude of risk of outcomes against the extent of hemorrhage. Further, while some outcome variables, such as arrhythmias and stroke, showed a trend, they did not reach statistical significance, suggesting limitation in sample size.

Rapid elevation in cardiac troponin-I in the early hours of reperfusion reflects hemorrhagic transformation of ST-elevation MI. Accurate diagnosis of hemorrhagic STEMI is possible from hourly thresholds of post reperfusion [hs-cTn-I] within the first 24 hours of reperfusion. Reperfused STEMI patients who develop intramyocardial hemorrhage carry significantly increased likelihood of adverse acute clinical outcomes and are most populated in the “Heart Failure Belt” of the United States.

Clinical Perspective: Pre-reperfusion [hs-cTn-I]identifies an ongoing MI but post-reperfusion [hs-cTn-I]can be used to diagnose hemorrhagic transformation of reperfused STEMI. Hemorrhagic STEMI carries multi-fold greater risk of in-hospital mortality, 30-day mortality and acute heart failure compared to non-hemorrhagic STEMI. Blood troponin-based biomarker warrants future use in clinical trials to mitigate intramyocardial hemorrhage and improve post-STEM 1 survival.

Additional Results from Clinical Studies:

Stable/Unstable Angina, Reinfarction and Stroke Risk 30-days post PCI in MIRON-ACUTE Stable Angina: Patients with a history of hMI displayed a insignificant odds of 30-day readmission (Adj. OR 1.029, 95% CI 0.66-1.606), while each year of age conferred a protective 2% reduction in angina risk (Adj. OR 0.964, 95% CI 0.996-0.016). Male gender exhibited a non-significant 19.7% increase in angina risk (Adj. OR 1.197, 95% CI 0.767-1.869). HTN was associated with a modest 32.4% reduction in angina risk (Adj. OR 0.676, 95% CI 1.195-0.294), whereas diabetes showed no substantial impact (Adj. OR 1.1, 95% CI 1.738-0.404). Dyslipidemia was linked to a 29.9% increase in the likelihood of angina (Adj. OR 1.294, 95% CI 2.154-0.86), and a smoking history significantly increased angina risk by 51.4% (Adj. OR 1.514, 95% CI 2.684-0.66). In the context of pre-existing medications, ACE (−)/ARB demonstrated a marked reduction in angina risk (OR 2.997, 95% CI 8.221-1.905), while beta-blockers conveyed a 68.9% risk reduction (Adj. OR −1.1665, 95% CI 0.958-0.21). Notably, aspirin and P2Y12 inhibitors exhibited no significant impact on angina risk, with adjusted odds ratios of −1.2494 (95% CI 0.824-0.187) and −0.1376 (95% CI 1.629-0.405), respectively. Unstable Angina: Patients with unstable angina who had a history of hMI were equally likely to be readmitted for UA compared to non-hMI within 30 days (Adj. OR 1.033, 95% CI 0.725-1.472). Additionally, increasing age was inversely associated with UA readmission risk (Adj. OR 0.97, 95% CI 0.956-0.985). Male gender was found to be a predictor of UA (Adj. OR 1.498, 95% CI 0.332-0.498), while a history of HTN showed a modest protective effect (Adj. OR 0.413, 95% CI 1.164-0.281). Diabetes displayed a non-significant impact on readmission UA risk (Adj. OR 0.733, 95% CI 1.666-0.372). Dyslipidemia showed a trend towards increased readmission UA risk (Adj. OR 0.981, 95% CI 2.441-0.566), while smoking history was not a statistically significant predictor (Adj. OR 0.86, 95% CI 2.139-0.496). Among pre-existing medications, the use of ACE (−)/ARB was significantly associated with a reduced risk of readmission UA (Adj. OR 0.4341, 95% CI 5.089-1.245), while beta-blockers showed a protective trend (Adj. OR 0.4294, 95% CI 1.069-0.262). Conversely, the use of antiplatelet agents, such as aspirin (Adj. OR 0.4412, 95% CI 1.229-0.3) and P2Y12 inhibitors (Adj. OR 0.2905, 95% CI 1.218-0.299), did not significantly impact readmission UA risk. Notably, interventional therapies, including GP IIb/IIIa receptor inhibitors, aspirin (given during percutaneous coronary intervention), and certain discharge medications such as beta-blockers and statins displayed mixed associations with UA readmission risk.

Re-infarction: patients with hMI did not exhibit a significantly different risk of reinfarction compared to those without hMI, with Adj. OR of 0.993 (95% CI 0.563-1.752). While increasing age demonstrated a subtle protective trend, with a 2% risk reduction per year (Adj. OR 0.976, 95% CI 0.957-0.996), gender, hypertension and diabetes did not show substantial impacts. Dyslipidemia was associated with a 55.2% increase in reinfarction risk (Adj. OR 1.552, 95% CI 2.891-0.719), and a history of smoking displayed a 10.1% elevated risk (Adj. OR 1.101, 95% CI 1.935-0.475).

Regarding pre-existing medications, beta-blockers exhibited a 5.6% lower risk of reinfarction (Adj. OR 0.944, 95% CI 2.626-0.605), while aspirin and P2Y12 inhibitors displayed no substantial impact. The study further explored medications administered PCI procedures, which yielded variable associations with reinfarction risk.

CVA-Stroke: hMI moderately impacted the risk of CVA, as the Adj. OR was 1.37 (95% CI 0.427-4.397), indicating no substantial difference in CVA risk between the two groups. However, it's crucial to acknowledge the study's limitation, namely the relatively low number of CVA cases, with only 14 occurrences, making it challenging to draw robust conclusions. The number of days since admission did not exhibit a substantial influence on CVA risk, with an Adj. OR of 1.011 (95% CI 0.97-1.054) per day, emphasizing the stability of CVA risk over time. Gender, hypertension, diabetes, dyslipidemia and smoking history did not demonstrate substantial effects on CVA risk, with Adj. ORs of 0.447 (95% CI 0.118-1.694), 1.139 (95% CI 0.309-4.208), 0.634 (95% CI 0.166-2.425), 0.41 (95% CI 0.126-1.339), and 1.404 (95% CI 0.714-2.76), respectively.

### MIRON-National—Detailed Account of 30-Day Acute Outcomes

### 30-Day Re-Hospitalization:

We observed a 10% readmission rate overall, with 12% among those with hemorrhagic MI (hMI) compared to 10% in the non-hMI group. The multivariable logistic regression analysis revealed several significant associations with readmission. hMI demonstrated a notable impact, with Adj. OR of 1.28 (95% CI 1.17-1.41, p<0.001), signifying a 28% higher likelihood of readmission compared to non-hMI patients. Age was also a significant predictor, with an Adj. OR of 1.01 (95% CI 1.01-1.02, p<0.001), indicating a 1% increase in the odds of readmission per year. Male gender exhibited a protective effect, with an Adj. OR of 0.73 (95% CI 0.67-0.80, p<0.001), suggesting a 27% lower likelihood of readmission compared to females. Diabetes (Adj. OR 1.36, 95% CI 1.25-1.49, p<0.001) and smoking (Adj. OR 0.92, 95% CI 0.81-1.03, p=0.15) were also associated with readmission. Hypertension and dyslipidemia did not show significant associations.

30-day Arrhythmia: Arrhythmia occurred in 3% of the overall cohort, with a higher prevalence observed in the hMI group (4%) compared to the non-hMI group (2%). Notably, hMI emerged as a significant predictor, exhibiting Adj. OR of 1.48 (95% CI 1.25-1.74, p<0.001), indicating a 48% increased likelihood of arrhythmia in patients with hMI compared to those without. Age was also a significant contributor, with an Adj. OR of 1.04 (95% CI 1.04-1.05, p<0.001), signifying a 4% higher odds of arrhythmia per year. Conversely, hypertension demonstrated a protective effect, with an Adj. OR of 0.76 (95% CI 0.65-0.89, p=0.001), suggesting a 24% lower likelihood of arrhythmia in hypertensive individuals. Other variables, including gender, diabetes, dyslipidemia, and smoking, exhibited varying degrees of association with arrhythmia. Our findings underscore the substantial impact of hMI on the occurrence of arrhythmia in acute phase post-MI.

30-day Acute heart Failure: Among the total cohort, 3% experienced AHF, with 4% in the hMI group and 2% in the non-hMI group. Notably, patients with hMI demonstrated a substantial 70.1% increase in the odds of AHF compared to non-hMI cases (Adj. OR=1.701, 95% CI [1.447, 1.995], p<0.001). Age exhibited a consistent positive association, with a 3.8% increase in AHF odds per additional year (Adj. OR=1.038, 95% CI [1.032, 1.045], p<0.001). Male gender showed a protective effect, with a 30.7% decrease in AHF odds (Adj. OR=0.690, 95% CI [0.590, 0.810], p<0.001). Hypertension was associated with a 39.3% reduction in AHF odds (Adj. OR=0.607, 95% CI [0.518, 0.710], p<0.001), while diabetes significantly increased the odds by 80.9% (Adj. OR=1.810, 95% CI [1.548, 2.115], p<0.001). Dyslipidemia and smoking did not reach statistical significance.

30-day CVA (Stroke): The presence of hMI did not demonstrate a statistically significant association with stroke incidence (Adj. OR=1.134, 95% CI [0.673, 1.838], p=0.622). However, age showed a significant positive association, contributing to a 2.8% increase in stroke odds for each additional year (Adj. OR=1.028, 95% CI [1.010, 1.047], p=0.004). Gender (Male) did not significantly impact stroke odds (Adj. OR=1.023, 95% CI [0.638, 1.684], p=0.927), and hypertension also did not show a statistically significant association (Adj. OR=1.251, 95% CI [0.784, 2.040], p=0.357). In contrast, diabetes exhibited a significant risk association, with a 67.1% increase in stroke odds (Adj. OR=1.671, 95% CI [1.060, 2.623], p=0.026). Dyslipidemia (Adj. OR=0.861, 95% CI [0.540, 1.396], p=0.536) and smoking (Adj. OR=1.114, 95% CI [0.582, 1.974], p=0.727) did not demonstrate statistically significant associations.

### Example 2. Length of Index Hospitalization for Hemorrhagic MI

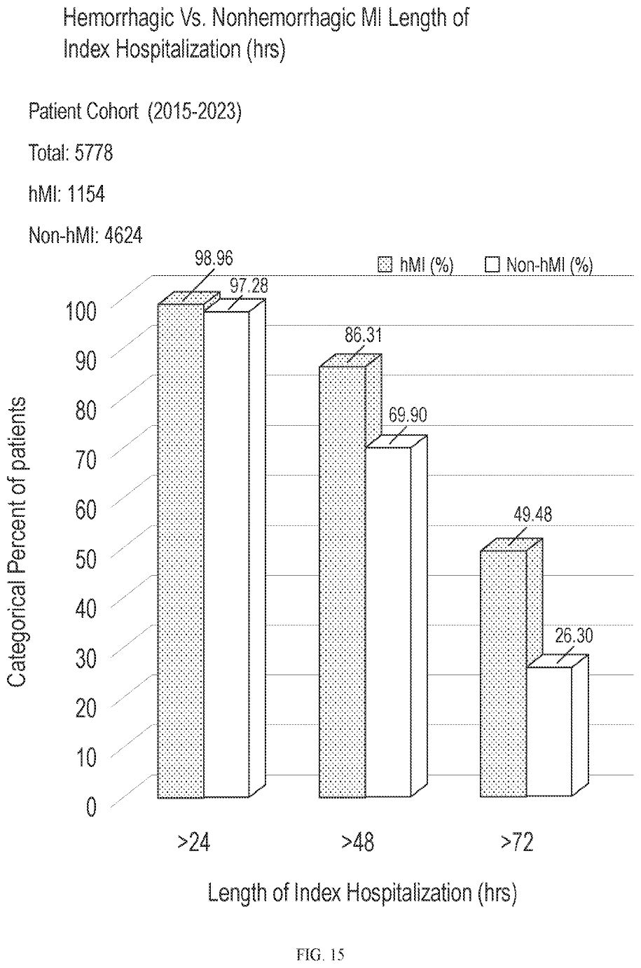

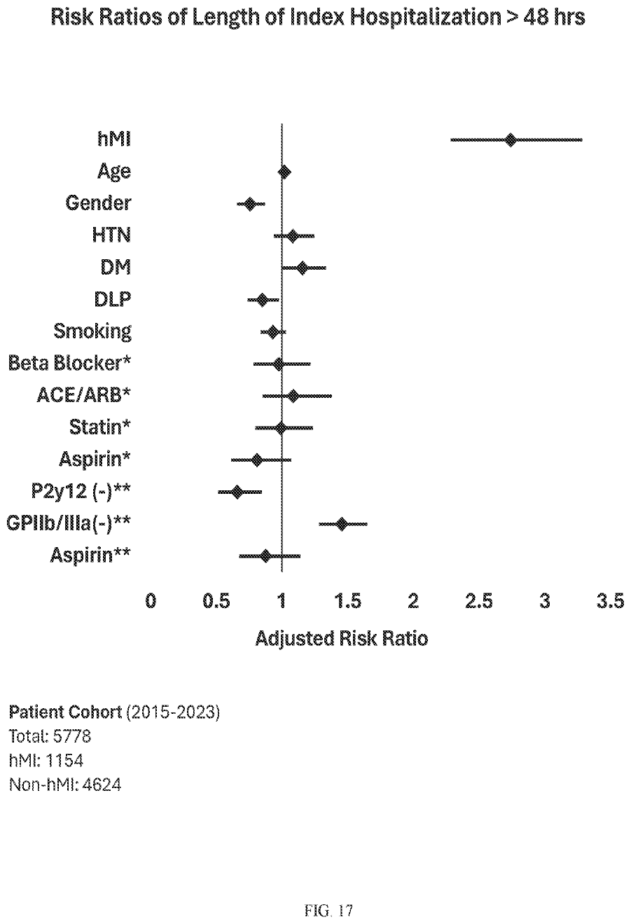

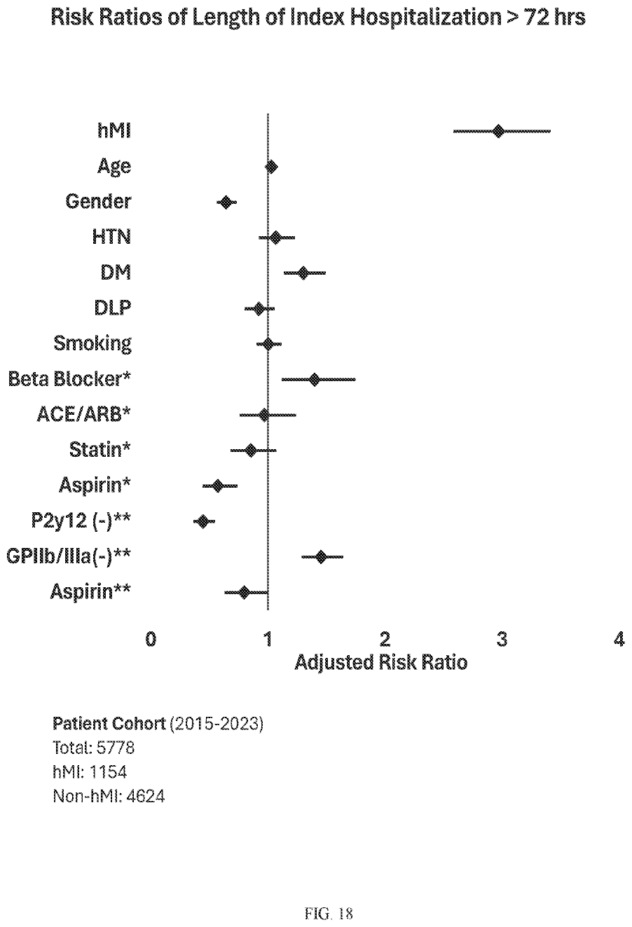

FIGS. 15-18 and Tables 4A-4C show that patients who were proven hemorrhagic in MIRON-ACUTE study had a longer hospitalization than the counterparts.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

TABLE 1A
Baseline characteristics of STEMI Patients.

| | Total cohort | | IMH+ | | IMH− | | |
|---|---|---|---|---|---|---|---|
| | (n = 120) | | (n = 51) | | (n = 69) | | p value |
| Age | 55.06 ± 10.19 | | 54.50 ± 11.01 | | 55.62 ± 9.38 | | 0.34 |
| Male gender, n (%) | 99 | (82.5) | 43 | (90.7) | 56 | (79.59) | 0.31 |

| | | | | |
|---|---|---|---|---|
| Body mass index (kg/m²) | 26.24 ± 4.41 | 26.59 ± 4.16 | 25.88 ± 4.76 | 0.30 |

Comorbidities, n (%)

| | | | | | | |
|---|---|---|---|---|---|---|
| Hypertension | 39 | (32.50) | 20 | (39.22) | 19 | (27.54) |
| Diabetes | 35 | (29.17) | 26 | (50.98) | 9 | (13.04) |
| Dyslipidemia | 48 | (40.0) | 26 | (50.98) | 22 | (31.88) |
| Smoking | 67 | (55.83) | 37 | (72.55) | 30 | (43.48) |

| | | | | |
|---|---|---|---|---|
| Heart rate (bpm) | 85.45 ± 20.92 | 86.25 ± 13.72 | 84.66 ± 28.11 | 0.22 |
| Systolic blood pressure (mmHg) | 136.33 ± 34.40 | 131.34 ± 39.49 | 141.33 ± 29.32 | 0.19 |
| Diastolic blood pressure (mmHg) | 92.08 ± 14.84 | 88.51 ± 10.87 | 95.66 ± 18.81 | 0.13 |
| Symptom onset to reperfusion (hrs) | 5.86 ± 3.8 | 5.71 ± 3.18 | 6.01 ± 4.43 | 0.19 |

Infarct-related artery, n (%)

| | | | | | | |
|---|---|---|---|---|---|---|
| Left anterior descending (LAD) | 81 | (67.50) | 46 | (90.20) | 35 | (50.72) |
| Left circumflex (LCx) | 18 | (15.0) | 5 | (9.80) | 13 | (18.84) |
| Right coronary artery (RCA) | 22 | (18.33) | 10 | (19.61) | 12 | (17.39) |

Single/Multi-vessel Disease, n (%)

| | | | | | | |
|---|---|---|---|---|---|---|
| 0-1 | 71 | (59.17) | 32 | (65.31) | 33 | (67.34) |
| 2-3 | 49 | (40.83) | 11 | (22.45) | 16 | (32.66) |
| TIMI flow grade 3 after PCI, n (%) | 75 | (62.5) | 32 | (74.41) | 43 | (87.76) |

hs-cTn-I (average ± SD)

| | | | |
|---|---|---|---|
| Pre-PCI Baseline (ng/ml) | 33.08 ± 29.02 | 10.96 ± 17.15 | 0.02 |
| Post-PCI Peak Level (ng/ml) | 348.16 ± 264.74 | 30.95 ± 29.67 | <0.0001 |
| Reperfusion Time to Peak Level (hr) | 4.02 ± 2.14 | 10.52 ± 5.57 | 0.008 |
| Peak Velocity (ng/ml/hr) | 218.55 ± 188.91 | 10.87 ± 17.56 | <0.0001 |
| Reperfusion Time to Peak Velocity (hr) | 0.89 ± 1.18 | 7.56 ± 13.85 | 0.002 |

CMR

| | | | | |
|---|---|---|---|---|
| BSA (m²) | 1.84 ± 0.19 | 1.87 ± 0.23 | 1.81 ± 0.15 | 0.05 |
| LV Mass (gm) | 100.41 ± 27.24 | 103.93 ± 30.13 | 96.88 ± 24.36 | 0.14 |
| Infarct Size (% LV) | 29.91 ± 16.81 | 42.77 ± 19.98 | 17.06 ± 13.63 | <0.05 |
| MVO Vol (% LV) | 6.76 ± 10.39 | 12.05 ± 9.16 | 1.47 ± 2.46 | <0.05 |
| IMH Vol (gm) | 10.38 ± 5.44 | 10.38 ± 5.44 | 0 | <0.05 |
| IMH Vol (% LV) | 8.57 ± 4.78 | 8.57 ± 4.78 | 0 | <0.05 |
| LVEF (%) | 37.55 ± 8.33 | 30.68 ± 7.45 | 44.43 ± 9.21 | <0.05 |
| RVEF (%) | 56.5 ± 9.05 | 58.54 ± 8.61 | 54.46 ± 9.49 | 0.35 |

IMH+: hemorrhagic MI;
IMH−: non-hemorrhagic MI;
hs-cTn-I: high-sensitivity troponin I

TABLE 1B
Patient Characteristics of MIRON-ACUTE

| | Hemorrhagic MI | | Non-hemorrhagic MI | | |
|---|---|---|---|---|---|
| Clinical Characteristics | (n = 1323) | | (n = 4857) | | p-value |
| Median age (IQR) - yr | 60.53 | (52.63-69.40) | 60.21 | (52.11-69.17) | 0.2171 |
| Male gender, n (%) | 1007 | (76.11%) | 3355 | (69.08%) | <0.0001 |
| Body mass index (kg/m²) | 29.87 | (6.38) | 30.35 | (7.77) | 0.0233 |

Co-morbid Conditions, n (%)

| | | | | | |
|---|---|---|---|---|---|
| Hypertension | 880 | (66.52%) | 3253 | (66.98%) | 0.7526 |
| Diabetes | 388 | (29.33%) | 1397 | (28.76%) | 0.6879 |
| Dyslipidemia | 756 | (57.14%) | 2945 | (60.63%) | 0.0216 |
| Smoking | | | | | 0.7363 |
| Never | 986 | (74.53%) | 3578 | (73.67%) | |
| Former | 108 | (8.16%) | 393 | (8.09%) | |
| Current | 229 | (17.31%) | 886 | (18.24%) | |
| Current vs Not Current | 229 | (17.31%) | 886 | (18.24%) | 0.4342 |
| Peripheral Vascular Disease | 102 | (7.71%) | 370 | (7.62%) | 0.9112 |

Cath Parameters

| | | | | | |
|---|---|---|---|---|---|
| Median systolic blood pressure | 141.00 | (122.00-159.00) | 142.0 | (123.0-161.0) | 0.4479 |
| (IQR) - mm Hg | | | | | |
| Median heart rate (IQR) - bpm | 102.50 | (77.00-115.00) | 92.00 | (62.00-112.00) | 0.4841 |
| Door to Balloon Time (hrs) | 0.87 | (1.15) | 0.87 | (1.12) | 0.8838 |
| LAD | 673 | (50.87%) | 1736 | (35.74%) | <0.0001 |
| LCx | 207 | (15.65%) | 624 | (12.85%) | 0.0082 |
| RCA | 403 | (30.46%) | 2419 | (49.80%) | <0.0001 |
| RAMUS | 10 | (0.76%) | 46 | (0.95%) | 0.5152 |
| LM | 29 | (2.19)% | 29 | (0.60%) | <0.0001 |
| Single Vessel Disease | 950 | (71.81%) | 3635 | (74.84%) | 0.026 |
| Multivessel Disease | 373 | (28.19%) | 1222 | (25.16%) | 0.026 |
| TIMI Flow | | | | | |
| TIMI flow grade 0 Before PCI, n (%) | 1010 | (76.63%) | 2671 | (55.45%) | <0.0001 |
| TIMI flow grade 3 Before PCI, n (%) | 168 | (12.75%) | 1099 | (22.82%) | <0.0001 |
| TIMI 0, 1, 2 vs TIMI 3 before PCI | 1155 | (87.30%) | 3758 | (77.37%) | <0.0001 |
| TIMI flow grade 0 After PCI, n (%) | 17 | (1.30%) | 46 | (0.96%) | 0.2846 |
| TIMI flow grade 3 After PCI, n (%) | 1232 | (94.12%) | 4639 | (96.93%) | 0.0004 |
| TIMI 0, 1, 2 vs TIMI 3 after PCI | 91 | (6.88%) | 218 | (4.49%) | 0.0004 |
| Post-PCI Peak Level (ng/ml) | 115.5 | (95.05) | 27.34 | (24.34) | <0.0001 |
| LVEF (%) | 48.22% | (13.92%) | 54.73% | (13.12%) | 0.0034 |
| Medications prior to procedure | | | | | |
| Beta Blockers | 152 | (11.49%) | 577 | (11.88%) | 0.6961 |
| ACE-Inhibitor | 65 | (4.91%) | 263 | (5.41%) | 0.4704 |
| ARB | 57 | (4.31%) | 141 | (2.90%) | 0.0101 |
| Statin | 186 | (14.06%) | 715 | (14.72%) | 0.5452 |
| Aspirin | 489 | (36.96%) | 1793 | (36.92%) | 0.9757 |
| Medications during procedure | | | | | |
| P2Y12 Inhibitor | 1172 | (88.59%) | 4435 | (91.31%) | 0.0024 |
| Anticoagulants | 1308 | (98.87%) | 4799 | (98.81% | 0.857 |
| Parenteral | 1100 | (83.14%) | 3942 | (81.16%) | 0.099 |
| GP IIb/IIIa | 650 | (49.13% | 2066 | (42.54%) | <0.0001 |
| Medications on discharge | | | | | |
| Beta Blockers | 1057 | (79.89%) | 4179 | (86.04%) | <0.0001 |
| ACE-Inhibitor | 666 | (50.34%) | 2588 | (53.28%) | 0.0573 |
| ARB | 165 | (12.47%) | 598 | (12.31%) | 0.8757 |
| Statin | 1144 | (86.47%) | 4482 | (92.28%) | <0.0001 |
| Aspirin | 1117 | (84.43%) | 4481 | (92.26%) | <0.0001 |
| P2Y12 Inhibitor | 1151 | (87%) | 4535 | (93.37%) | <0.0001 |
| Oral Anticoagulant | 55 | (4.16%) | 168 | (3.46%) | 0.2273 |

TABLE 1C
Clinical characteristics of patient population in MIRON-NATIONAL

| | | Overall | | Non-hMI, n (%) | | hMI, n (%) | | |
|---|---|---|---|---|---|---|---|---|
| | N | N = 24,181 | | N = 18,425 | | N = 5,756 | | p-value |
| Race | 24,181 | | | | | | | 0.1 |
| White | | 19,338 | (80%) | 14,789 | (80%) | 4,549 | (79%) | |
| Asian | | 583 | (2.4%) | 428 | (2.3%) | 155 | (2.7%) | |
| Black | | 1,520 | (6.3%) | 1,131 | (6.1%) | 389 | (6.8%) | |
| Other/Unknown | | 2,740 | (11%) | 2,077 | (11%) | 663 | (12%) | |
| Ethnicity | 24,064 | | | | | | | 0.3 |
| Hispanic | | 2,199 | (9.1%) | 1,656 | (9.0%) | 543 | (9.5%) | |
| Not Hispanic | | 21,865 | (91%) | 16,669 | (91%) | 5,196 | (91%) | |
| Unknown | | 117 | | 100 | | 17 | | |
| Gender | 24,181 | | | | | | | 0.001*** |
| Female | | 6,877 | (28%) | 5,438 | (30%) | 1,439 | (25%) | |
| Male | | 17,299 | (72%) | 12,985 | (70%) | 4,314 | (75%) | |
| Co-morbidities | | | | | | | | |
| Peripheral Artery | 24,181 | | | | | | | 0.7 |
| Disease | | | | | | | | |
| 0 | | 23,141 | (96%) | 17,628 | (96%) | 5,513 | (96%) | |
| 1 | | 1,040 | (4.3%) | 797 | (4.3%) | 243 | (4.2%) | |
| Smoking | 24,181 | | | | | | | 0.001*** |
| None | | 20,081 | (83%) | 15,349 | (83%) | 4,732 | (82%) | |
| Active | | 2,985 | (12%) | 2,190 | (12%) | 795 | (14%) | |
| Former | | 1,115 | (4.6%) | 886 | (4.8%) | 229 | (4.0%) | |
| Hypertension | 24,181 | | | | | | | 0.001*** |
| 0 | | 10,089 | (42%) | 7,562 | (41%) | 2,527 | (44%) | |
| 1 | | 14,092 | (58%) | 10,863 | (59%) | 3,229 | (56%) | |
| Diabetes | 24,181 | | | | | | | 0.1 |
| 0 | | 16,342 | (68%) | 12,503 | (68%) | 3,839 | (67%) | |
| 1 | | 7,839 | (32%) | 5,922 | (32%) | 1,917 | (33%) | |
| Dyslipidemia | 24,181 | | | | | | | 0.7 |
| 0 | | 9,318 | (39%) | 7,089 | (38%) | 2,229 | (39%) | |
| 1 | | 14,863 | (61%) | 11,336 | (62%) | 3,527 | (61%) | |

TABLE 2
Diagnostic Performance of [hs-cTn-I] in human patients
in Determining Hemorrhagic MI: Comparison of sensitivity,
specificity, positive predictive value (PPV), negative predictive
value (NPV), and area under the curve (AUC) from receiver
operating characteristics analysis with optimal [hs-cTn-
I] cut-offs at pre-PCI and post reperfusion time points.

| | [hs-cTn-I] | | | | | |
|---|---|---|---|---|---|---|
| Time after | Cut-off | | | | | |
| PCI (hrs) | (ng/ml) | Sensitivity | Specificity | PPV | NPV | AUC |
| −1* | 11.38 | 47% | 72% | 0.40 | 0.77 | 0.54 |
| 0 | 44.68 | 87% | 77% | 0.61 | 0.94 | 0.88 |
| 1 | 92.68 | 94% | 89% | 0.78 | 0.97 | 0.95 |
| 2 | 91.17 | 94% | 85% | 0.73 | 0.97 | 0.94 |
| 3 | 114.64 | 90% | 89% | 0.78 | 0.96 | 0.94 |
| 4 | 104.31 | 90% | 89% | 0.78 | 0.96 | 0.94 |
| 5 | 100.16 | 90% | 87% | 0.74 | 0.96 | 0.93 |
| 6 | 93.45 | 90% | 87% | 0.74 | 0.96 | 0.93 |
| 7 | 92.55 | 90% | 87% | 0.74 | 0.96 | 0.93 |
| 8 | 81.97 | 90% | 87% | 0.74 | 0.96 | 0.93 |
| 9 | 85.39 | 90% | 89% | 0.78 | 0.96 | 0.93 |
| 10 | 86.97 | 84% | 88% | 0.74 | 0.93 | 0.92 |
| 11 | 77.27 | 87% | 87% | 0.73 | 0.94 | 0.92 |
| 16 | 63.51 | 87% | 87% | 0.73 | 0.94 | 0.92 |
| 20 | 40.33 | 94% | 81% | 0.67 | 0.97 | 0.91 |
| 24 | 32.04 | 81% | 81% | 0.64 | 0.91 | 0.84 |
| 48 | 19.24 | 81% | 80% | 0.63 | 0.91 | 0.85 |

*denotes pre-PCI time point, i.e., baseline

TABLE 3
Regional distribution of STEMI with number
of patients and proportion nationwide.

| Zip | Overall, n (%) | | Non-hMI, n (%) | | hMI, n (%) | |
|---|---|---|---|---|---|---|
| Code | N = 24,181 | | N = 18,425 | | N = 5,756 | |
| 0 | 1,794 | (7.8%) | 1,400 | (8.0%) | 394 | (7.2%) |
| 1 | 620 | (2.7%) | 527 | (3.0%) | 93 | (1.7%) |
| 2 | 2,627 | (11%) | 1,906 | (11%) | 721 | (13%) |
| 3 | 1,096 | (4.8%) | 757 | (4.3%) | 339 | (6.2%) |
| 4 | 5,522 | (24%) | 4,320 | (25%) | 1,202 | (22%) |
| 5 | 1,424 | (6.2%) | 1,061 | (6.0%) | 363 | (6.6%) |
| 6 | 2,687 | (12%) | 2,006 | (11%) | 681 | (12%) |
| 7 | 1,096 | (4.8%) | 782 | (4.5%) | 314 | (5.7%) |
| 8 | 1,620 | (7.0%) | 1,142 | (6.5%) | 478 | (8.7%) |
| 9 | 4,558 | (20%) | 3,669 | (21%) | 889 | (16%) |

| | | | |
|---|---|---|---|
| Unknown | 1,137 | 855 | 282 |

TABLE 4A
Risk Ratios of Length of Index Hospitalization >24 hrs.

| | | | |
|---|---|---|---|
| | Risk | 95% CI | |
| Variables | Ratio | (Lower-Upper) | p-VALUE |

| | | | | |
|---|---|---|---|---|
| hMI | 2.709 | 1.49 | 4.926 | 0.0011 |
| Age | 0.989 | 0.975 | 1.003 | 0.1293 |
| Gender | 0.803 | 0.544 | 1.185 | 0.2683 |
| HTN | 1.324 | 0.883 | 1.986 | 0.1749 |
| DM | 0.756 | 0.516 | 1.109 | 0.1524 |
| DLP | 0.806 | 0.538 | 1.207 | 0.2946 |
| Smoking | 0.835 | 0.642 | 1.086 | 0.1783 |
| Beta Blocker* | 1.066 | 0.611 | 1.862 | 0.8211 |
| ACE/ARB* | 1.05 | 0.568 | 1.939 | 0.8773 |
| Statin* | 1.181 | 0.683 | 2.039 | 0.5517 |
| Aspirin* | 1.218 | 0.642 | 2.31 | 0.5455 |
| P2y12 (−)** | 1.865 | 1.102 | 3.156 | 0.0202 |
| GPIIb/IIIa(−)** | 1.437 | 0.997 | 2.07 | 0.0518 |
| Aspirin** | 2.043 | 1.107 | 3.769 | 0.0222 |

TABLE 4B
Risk Ratios of Length of Index Hospitalization >48 hrs.

| | | | |
|---|---|---|---|
| | Risk | 95% CI | |
| Variables | Ratio | (Lower-Upper) | p-VALUE |

| | | | | |
|---|---|---|---|---|
| hMI | 2.739 | 2.284 | 3.284 | <.0001 |
| Age | 1.017 | 1.012 | 1.022 | <.0001 |
| Gender | 0.756 | 0.659 | 0.868 | <.0001 |
| HTN | 1.081 | 0.939 | 1.245 | 0.2802 |
| DM | 1.156 | 1.003 | 1.332 | 0.0447 |
| DLP | 0.849 | 0.739 | 0.975 | 0.0205 |
| Smoking | 0.929 | 0.838 | 1.03 | 0.1645 |
| Beta Blocker* | 0.975 | 0.782 | 1.217 | 0.8235 |
| ACE/ARB* | 1.084 | 0.852 | 1.38 | 0.5101 |
| Statin* | 0.991 | 0.796 | 1.234 | 0.9382 |
| Aspirin* | 0.809 | 0.612 | 1.069 | 0.1365 |
| P2y12 (−)** | 0.659 | 0.514 | 0.846 | 0.0011 |
| GPIIb/IIIa(−)** | 1.454 | 1.284 | 1.647 | <.0001 |
| Aspirin** | 0.875 | 0.673 | 1.138 | 0.3199 |

TABLE 4C
Risk Ratios of Length of Index Hospitalization >72 hrs.

| | | | |
|---|---|---|---|
| | Risk | 95% CI | |
| Variables | Ratio | (Lower-Upper) | p-VALUE |

| | | | | |
|---|---|---|---|---|
| hMI | 2.968 | 2.284 | 3.284 | <.0001 |
| Age | 1.028 | 1.012 | 1.022 | <.0001 |
| Gender | 0.643 | 0.659 | 0.868 | <.0001 |
| HTN | 1.067 | 0.939 | 1.245 | 0.2802 |
| DM | 1.302 | 1.003 | 1.332 | 0.0447 |
| DLP | 0.922 | 0.739 | 0.975 | 0.0205 |
| Smoking | 1.003 | 0.838 | 1.03 | 0.1645 |
| Beta Blocker* | 1.398 | 0.782 | 1.217 | 0.8235 |
| ACE/ARB* | 0.969 | 0.852 | 1.38 | 0.5101 |
| Statin* | 0.854 | 0.796 | 1.234 | 0.9382 |
| Aspirin* | 0.572 | 0.612 | 1.069 | 0.1365 |
| P2y12 (−)** | 0.445 | 0.514 | 0.846 | 0.0011 |
| GPIIb/IIIa(−)** | 1.453 | 1.284 | 1.647 | <.0001 |
| Aspirin** | 0.796 | 0.673 | 1.138 | 0.3199 |

What is claimed is:

1. A method of treating a human subject in need thereof, wherein the human subject undergoes a reperfusion therapy following myocardial ischemia, the method comprising:
administering a treatment therapy to the human subject, wherein the human subject is detected with one or more of:
a level of troponin-I being at least 90 ng/mL in a blood sample obtained between 0.5 hour and 7.5 hours after the reperfusion therapy,
a level of the troponin-I being at least 75 ng/mL in a blood sample obtained between 7.5 hours and 11.5 hours after the reperfusion therapy,
a level of the troponin-I being at least 60 ng/mL in a blood sample obtained between 11.5 hours and 16.5 hours after the reperfusion therapy,
a level of the troponin-I being at least 40 ng/mL in a blood sample obtained between 16.5 hours and 20.5 hours after the reperfusion therapy, and
a level of the troponin-I being at least 30 ng/mL in a blood sample obtained between 20.5 hours and 24.5 hours after the reperfusion therapy,
optionally with the understanding that the detection of said one or more levels of the troponin-I at said one or more times indicates the human subject has hemorrhagic myocardial infarction after the reperfusion therapy,
wherein the treatment therapy comprises an iron chelator, an agent that binds or regulates heme, and/or an anti-inflammatory agent.

2. A method of treating a human subject in need thereof, wherein the human subject undergoes a reperfusion therapy following myocardial ischemia, the method comprising:
administering a treatment therapy to the human subject, wherein the subject is detected with a high level of troponin-I in a blood sample obtained between 3 hours and 5 hours following the reperfusion therapy, wherein the high level of the troponin-I is one or both of:
at least 300 ng/mL or within 348±37 ng/mL, and
a concentration at least 10 times that obtained between 9.5 and 11.5 hours after a reperfusion therapy in a control human subject, wherein the control human subject does not have hemorrhagic myocardial infarction after the reperfusion therapy following a myocardial infarction,
optionally with the understanding that the detection of said one or both high levels of the troponin-I between 3 hours and 5 hours following the reperfusion therapy indicates the human subject has hemorrhagic myocardial infarction after the reperfusion therapy, and
wherein the treatment therapy comprises an iron chelator, an agent that binds or regulates heme, and/or an anti-inflammatory agent.

3. A method of treating a human subject in need thereof, wherein the human subject undergoes a reperfusion therapy following myocardial ischemia, the method comprising:
administering a treatment therapy to the human subject, wherein the human subject is detected with a high rate of increase in troponin-I level comparing within first 2 hours after the reperfusion therapy relative to before the reperfusion therapy, preferably comparing within first 2 hours after the reperfusion therapy relative to within one hour or 30 minutes or 10 minutes before the reperfusion therapy, wherein the high rate of increase is:
being at least 190 ng/mL/hr or within 218.55±26.45 ng/mL/hr, and/or
being at least 10 times a rate of increase in a control human subject, wherein the rate of increase in the control human subject is comparing within 21 hours after a reperfusion therapy relative to immediately after the reperfusion therapy in the control human subject, and wherein the control human subject does not have hemorrhagic myocardial infarction after the reperfusion therapy following myocardial infarction,
optionally with the understanding that the detection of said one or both high rate of increase in the troponin-I between 0.5 hour and 2 hours following the reperfusion therapy indicates the human subject has hemorrhagic myocardial infarction after the reperfusion therapy,
wherein the treatment therapy comprises an iron chelator, an agent that binds or regulates heme, and/or an anti-inflammatory agent.

4. A method of treating a canine subject in need thereof, wherein the canine subject undergoes a reperfusion therapy following myocardial ischemia, the method comprising:
administering a treatment therapy to the canine subject, wherein the canine subject is detected with:
a level of troponin-I being at least 200 ng/mL or within 259.72±15.44 ng/mL in a blood sample obtained or in blood measured between 3 hours and 4 hours after the reperfusion,
a level of the troponin-I in a blood sample obtained or in blood measured between 3 hours and 4 hours after the reperfusion being at least 3.5 times that obtained between 15 hours and 19 hours after a reperfusion therapy in a control canine subject, wherein the control canine subject does not have hemorrhagic myocardial infarction after the reperfusion therapy following a myocardial infarction, and/or
a high rate of increase in the troponin-I comparing a blood sample obtained between 1.5 hours and 2 hours after the reperfusion relative to one immediately after the reperfusion therapy, wherein the high rate of increase is at least 60 ng/mL/hr and/or at least 10 times a rate of increase in the control canine subject, wherein the rate of increase in the control canine subject is comparing a blood sample obtained between 4 hours and 9 hours after the reperfusion therapy in the control canine subject relative to one immediately after the reperfusion therapy in the control canine subject,
optionally with the understanding that the detection of said one or both levels of the troponin-I and/or said high rate of increase in the troponin-I indicates the canine subject has hemorrhagic myocardial infarction after the reperfusion therapy, and
wherein the treatment therapy comprises an iron chelator, an agent that binds or regulates heme, and/or an anti-inflammatory agent.

5. The method of claim 2, wherein the human subject is detected with the high level of the troponin-I in the biological sample obtained at about 4 hours or between 3.5 and 4.5 hours after the reperfusion therapy.

6. The method of claim 3, wherein the human subject is detected with the high rate of increase in the troponin-I level in the biological sample obtained at about 1 hour following the reperfusion therapy.

7. The method of claim 1, wherein the level of the troponin-I detected in the biological sample obtained between 3 hours and 5 hours after the reperfusion is at least 250 ng/mL.

8. The method of claim 4, wherein the level of the troponin-I detected in the biological sample obtained between 3 hours and 4 hours after the reperfusion is at least 240 ng/mL.

9. The method of any one of claims 1-8, wherein the treatment therapy is administered immediately after detecting the level of the troponin-I indicating the presence of the hemorrhagic myocardial infarction in the subject, or administered within 3 hours after the detection.

10. The method of claim 9, wherein the treatment therapy comprises the iron chelator, and the iron chelator comprises a ferrous iron chelator, a ferric iron chelator, or both.

11. A method of examining a subject in need thereof, wherein the subject has myocardial ischemia and received a reperfusion therapy, the method comprising:
measuring a level of the troponin-I in a blood sample obtained at one or more time points between immediately following the reperfusion therapy and 24 hours following the reperfusion therapy from the subject, and/or
measuring a level of troponin-I noninvasively in the subject at one or more time points between immediately following the reperfusion therapy and 24 hours following the reperfusion therapy.

12. The method of claim 11, wherein the measuring comprises measuring in the blood samples at two or more time points between immediately following the reperfusion therapy and 4 hours following the reperfusion therapy, to obtain a rate of increase in the troponin-I level over the time lapse between the two or more time points.

13. The method of claim 11, wherein the measuring comprises measuring a time series of troponin-I levels in the blood samples obtained or measured noninvasively in the subject at three or more time points, and obtaining a highest level of the troponin-I from the time series.

14. The method of any one of claims 11-13, wherein the subject is a human subject indicated to have hemorrhagic myocardial infarction when the subject is detected with:
a level of the troponin-I being at least 90 ng/mL in a blood sample obtained or measured noninvasively from the subject between 0.5 hour and 7.5 hours after the reperfusion therapy,
a level of the troponin-I being at least 75 ng/mL in a blood sample obtained or measured noninvasively from the subject between 7.5 hours and 11.5 hours after the reperfusion therapy,
a level of the troponin-I being at least 60 ng/mL in a blood sample obtained or measured noninvasively from the subject between 11.5 hours and 16.5 hours after the reperfusion therapy,
a level of the troponin-I being at least 40 ng/mL in a blood sample obtained or measured noninvasively from the subject between 16.5 hours and 20.5 hours after the reperfusion therapy,
a level of the troponin-I being at least 30 ng/mL in a blood sample obtained or measured noninvasively from the subject between 20.5 hours and 24.5 hours after the reperfusion therapy,
a high level of the troponin-I being at least 300 ng/mL or within 348±37 ng/mL in a blood sample obtained or measured noninvasively from the subject between 3 hours and 5 hours after the reperfusion therapy,
a high level of the troponin-I in a blood sample obtained or measured noninvasively from the subject between 3 hours and 5 hours after the reperfusion therapy being at least 10 times that obtained or measured between 9.5 and 11.5 hours after a reperfusion therapy in a control human subject,
a high rate of increase in the troponin-I level comparing a blood sample obtained or measured noninvasively from the subject within 2 hours after the reperfusion therapy relative to one immediately after the reperfusion therapy, wherein the high rate of increase is at least 190 ng/mL/hr, or within 218.55±26.45 ng/mL/hr, and/or being at least 10 times a rate of increase in the control human subject, wherein the rate of increase in the control human subject is comparing within 21 hours after the reperfusion therapy relative to one immediately after the reperfusion therapy in the control human subject,
wherein the control human subject is a human who does not have hemorrhagic myocardial infarction after the reperfusion therapy following myocardial infarction;
and optionally the method further comprises prescribing or administering a treatment therapy to the human subject.

15. The method of any one of claims 11-13, wherein the subject is a canine subject, and the subject is indicated to have hemorrhagic myocardial infarction when the subject is detected with:
a level of the troponin-I being at least 200 ng/mL or within 259.72±15.44 ng/mL in a blood sample obtained or measured noninvasively from the subject between 3 hours and 4 hours after the reperfusion,
a level of the troponin-I in a blood sample obtained or measured noninvasively from the subject between 3 hours and 4 hours after the reperfusion being at least 3.5 times that between 15 hours and 19 hours after a reperfusion therapy in a control canine subject, wherein the control canine subject does not have hemorrhagic myocardial infarction after the reperfusion therapy following a myocardial infarction, and/or
a high rate of increase in the troponin-I comparing a blood sample obtained or measured noninvasively from the subject between 1.5 hours and 2 hours after the reperfusion relative to one immediately after the reperfusion therapy, wherein the high rate of increase is at least 60 ng/mL/hr and/or at least 10 times a rate of increase in the control canine subject, wherein the rate of increase in the control canine subject is comparing a blood sample obtained between 4 hours and 9 hours after the reperfusion therapy in the control canine subject relative to one obtained immediately after the reperfusion therapy in the control canine subject, and optionally the method further comprises prescribing or administering a treatment therapy to the subject.

16. The method of any one of claims 1-4, 11, and 15, wherein the troponin-I is cardiac troponin-I or a fragment thereof, and it is measured or detected using a monoclonal antibody specifically binding cardiac troponin-I.

17. A method for providing prognosis of a likelihood of survival about 30 days after receiving reperfusion therapy in a human subject with acute ST-elevation myocardial infarction (STEMI), and/or providing prognosis of a likelihood of requiring rehospitalization within about 30 days from receiving the reperfusion therapy in the human subject, the method comprising:
measuring a level of troponin-I in a blood sample obtained, or noninvasively, from the subject, at one or more time points following the reperfusion therapy,
wherein:
a level of troponin-I being at least 90 ng/mL in a blood sample obtained between 0.5 hour and 7.5 hours after the reperfusion therapy,
a level of the troponin-I being at least 75 ng/mL in a blood sample obtained between 7.5 hours and 11.5 hours after the reperfusion therapy,
a level of the troponin-I being at least 60 ng/mL in a blood sample obtained between 11.5 hours and 16.5 hours after the reperfusion therapy,
a level of the troponin-I being at least 40 ng/mL in a blood sample obtained between 16.5 hours and 20.5 hours after the reperfusion therapy,
a level of the troponin-I being at least 30 ng/mL in a blood sample obtained between 20.5 hours and 24.5 hours after the reperfusion therapy,
a level of the troponin-I being at least 300 ng/mL or within 348±37 ng/mL in a blood sample obtained between 3 hours and 5 hours after the reperfusion therapy, and/or
a level of the troponin-I in a blood sample obtained between 3 hours and 5 hours after the reperfusion therapy being at least 10 times that obtained between 9.5 and 11.5 hours after a reperfusion therapy in a control human subject, wherein the control human subject does not have hemorrhagic myocardial infarction after the reperfusion therapy following a myocardial infarction,
indicates that the human subject has a lower likelihood of survival at about the 30 days and a higher likelihood of requiring the rehospitalization within the about 30 days of the reperfusion, compared to the control human subject.

18. The method of claim 17, wherein the human subject is diagnosed with or assayed to determine hemorrhagic myocardial infarction.
